SEQUENCE LISTING (27 sequences)

SEQ ID NO: 1 - DNA, 569 bp, Artificial Sequence
  IRES-1
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 2 - DNA, 556 bp, Artificial Sequence
  IRES-2
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataa

SEQ ID NO: 3 - DNA, 549 bp, Artificial Sequence
  IRES-3
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacg

SEQ ID NO: 4 - DNA, 10309 bp, Artificial Sequence
  pCLD-BDDE-1A
ccaatgatcttaagttaatcgaatttgcagcccgggactagctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaacttaagatccgaccggacgcgttctattaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatgatgtgtgatcagttatctatgcggccgcggtggcggcgtcgaccgagaggttttccgatccggtcgatgcggactcgctcaggtccctcggtggcggagtaccgttcggaggccgacgggtttccgatccaagagtactggaaagaccgcgaagagtttgtcctcaaccgcgagctgtggaaaaaaaagggacaggataagtatgacatcatcaaggaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagaggatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaataggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagcgctattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtctaccggtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctacctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctcggatccgccgccaccgaattcatagataactgatccagtgcccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaacccacaaggagacgaccttccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgatctagatccccctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagggggatcttcgcgatactgcatcgatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatagacaccgcggtggagctccagcttttgttccctttagtgagggttaattagttcttaatacgactcactatagggcgaattggctaccgggccgcccatcgagggtatcataagcttttaaatcgatagatgcgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 10111 bp, Artificial Sequence
  pCLD-BDDE-1B
ccaatgatcttaagttaatcgaatttgcagcccgggactagctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaacttaagatccgaccggacgcgttctattaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatgatgtgtgatcagttatctatgcggccgcggtggcggcgtcgaccgagaggttttccgatccggtcgatgcggactcgctcaggtccctcggtggcggagtaccgttcggaggccgacgggtttccgatccaagagtactggaaagaccgcgaagagtttgtcctcaaccgcgagctgtggaaaaaaaagggacaggataagtatgacatcatcaaggaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagaggatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaataggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagcgctattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtctaccggtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctacctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctcggatccgccgccaccgaattcatagataactgatccagtgcccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctctagatccccctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagggggatcttcgcgatactgcatcgatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatagacaccgcggtggagctccagcttttgttccctttagtgagggttaattagttcttaatacgactcactatagggcgaattggctaccgggccgcccatcgagggtatcataagcttttaaatcgatagatgcgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 63 bp, Artificial Sequence
  5′ ITR
ccctagaaagataatcatattgtgacgtacgttaaagataatcatgcgtaaaattgacgcatg

SEQ ID NO: 7 - DNA, 35 bp, Artificial Sequence
  3′ ITR
catgcgtcaattttacgcagactatctttctaggg

SEQ ID NO: 8 - DNA, 1211 bp, Artificial Sequence
  HS4 Insulator
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 9 - DNA, 5837 bp, Artificial Sequence
  EASE
gaattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattctgtgcttagtaaaaggttgctttttatattttgagagaaatctatttaaagatcatggcccatattttgtgcatatttttttctgtataccatttccatatatgtgtgtgtgtgtacatatatgtatatatataaaatgttagaacatttgaggaaatagctaaaagtacaaaagtaatgttttctaattttttactccccgaggttatttcttttttccttgttttcctttctctttgttcctatcatcagtttctagtaatactcttattgaacagtgattattcaaatgtcacattatttattaatcagcatttaaatggtaaaaccagacagaccatactttctctgagtgatgacaacatccatttttagtaatgataaactagaagggtcaggcttgatagtctttgtcaggactaatttatagactgtaaaggccaaaagaaataagaaatgtcaaaactcttgtgaaactagacatacagatattaccaagagagaaactagaaaaaaaaattctgtgacatggccttaatttgccaggcaccatcgtgaaggcctaaacccctcttagaagctcactcagatgccatcctgcttctctgatgagacttcctgtcaatacaaacatggtttaggaagaatgagtgtttgcagtataaaccagttatttactagccttactttaagaatatactgtagtgtccttgagagagaaggtgtttgttttctgtaatttatgacccttttgaaaccatagatcagcacaaaggaactggggatatggaaatgggaacataacttaaatccagaaaagtgaatcagattccctgtgaggacaaaatgcaatatttagaaataggatctttaggctgggagggagaaaagaggaaaaaaatgaaagtataacatttttcataagtataagatttcataaaaaaatgaaatctataacatagagggtgttgataaagtaagcatggatatgtttagtaaagccgacagagctaagaattagctttgtgagtaattggacttaatcaaacttttcaaggtgggatacaaatgaataattgtagaataaatggataaaagaatatgaataaaatgaatagtgagtaaaaattaaaaatgaagctttttacttaagtgcatattgtagtggctagaacaaatagattcaaaatagaaatcatttatatattcttgattagaagataaaatgttattttagaaatagccatctttggaagtaaatttgctatgttgaacaaccaggttttcataatttgtctcttattttttttcaggaagaaaaaaaaacttgacttatttgtactgctaagttttattcaatgtgcttgcttgcttaaatttttaatgaagttttagtcatttggtggtcaaattccttttatctactaatcgcttttcgtggctttggcttttaaaattgtatttactgcatttatttgtgtgtattaggagtcaggtggccatatgtgccatggcatgtgtatggaagcacttgtggacatgaatcttctctttccacatgtgtgggccactggaatcaaactagtgtcaggcttggcagcaatttttaatgcactgagccttctcaccaccccctggacttttgtggggcagaaggggacaagtttaatattttatttactccatgtagaaagcctttaaaaaatgtagaaagcctttaaactacctattgttttatttgaattatgaagctcttgtgtttatataaattacagttaggtactgtggagactaatggtagctacaatagtaatattaatagctaaaacttagtagaatctgattgagttaatttggccctttccatcataaggtactcttcccaagcatcacatgacctgtgcttaagtctggtgggggcttatggctttgatattgaaaacaaatcgtcaaggatgttaatttcttgttactgctattacactgaattttctatggctctttaggagaggaagagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtct

SEQ ID NO: 10 - DNA, 280 bp, Artificial Sequence
  Beta-globin polyA
tgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaa

SEQ ID NO: 11 - DNA, 538 bp, Artificial Sequence
  hCMV immediate-early enhancer/promoter
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatc

SEQ ID NO: 12 - DNA, 366 bp, Artificial Sequence
  TPL
ctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctc

SEQ ID NO: 13 - DNA, 589 bp, Artificial Sequence
  pUC replication origin Ori
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 14 - DNA, 861 bp, Artificial Sequence
  Ampicillin resistance gene
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 15 - DNA, 1122 bp, Artificial Sequence
  GS gene
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 16 - DNA, 795 bp, Artificial Sequence
  Neomycin resistance gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctga

SEQ ID NO: 17 - DNA, 597 bp, Artificial Sequence
  Puromycin resistance gene
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 18 - DNA, 399 bp, Artificial Sequence
  Blasticidin resistance gene
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgctatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 19 - DNA, 222 bp, Artificial Sequence
  SV40 late polyA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 20 - DNA, 197 bp, Artificial Sequence
  SV40 promoter
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 21 - DNA, 237 bp, Artificial Sequence
  SV40 enhancer
gctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgctccatcg

SEQ ID NO: 22 - DNA, 9845 bp, Artificial Sequence
  pCLD-BDDE-2A
ccaatgatcttaagttaatcgaatttgcagcccgggactagctacttaagatccgaccggacgcgttctattaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatgatgtgtgatcagttatctatgcggccgcggtggcggcgtcgaccgagaggttttccgatccggtcgatgcggactcgctcaggtccctcggtggcggagtaccgttcggaggccgacgggtttccgatccaagagtactggaaagaccgcgaagagtttgtcctcaaccgcgagctgtggaaaaaaaagggacaggataagtatgacatcatcaaggaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagaggatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaataggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagcgctattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtctaccggtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctacctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctcggatccgccgccaccgaattcatagataactgatccagtgcccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaacccacaaggagacgaccttccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgatctagatccccctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagggggatcttcgcgatactgcatcgattagacaccgcggtggagctccagcttttgttccctttagtgagggttaattagttcttaatacgactcactatagggcgaattggctaccgggccgcccatcgagggtatcataagcttttaaatcgatagatgcgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 23 - DNA, 9647 bp, Artificial Sequence
  pCLD-BDDE-2B
ccaatgatcttaagttaatcgaatttgcagcccgggactagctacttaagatccgaccggacgcgttctattaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatgatgtgtgatcagttatctatgcggccgcggtggcggcgtcgaccgagaggttttccgatccggtcgatgcggactcgctcaggtccctcggtggcggagtaccgttcggaggccgacgggtttccgatccaagagtactggaaagaccgcgaagagtttgtcctcaaccgcgagctgtggaaaaaaaagggacaggataagtatgacatcatcaaggaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacagctggccctcgcagacagcgatgcggaagaggatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaataggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagcgctattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtctaccggtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctacctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctcggatccgccgccaccgaattcatagataactgatccagtgcccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctctagatccccctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagggggatcttcgcgatactgcatcgattagacaccgcggtggagctccagcttttgttccctttagtgagggttaattagttcttaatacgactcactatagggcgaattggctaccgggccgcccatcgagggtatcataagcttttaaatcgatagatgcgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 24 - DNA, 9757 bp, Artificial Sequence
  pCLD-SE
tcgaatttgcagcccgggactagctacttaagatccgaccggacgcgtactgagagcgctattctgaacttttcttttgttcccttcccttctaccacaccctaattgtaatccattttaatttcctggtcacagtcctgtctctccttccattgtaccttgcccttttctaaagagcgactgcaaagtatgtttgcgtaggtgaggatctaaaactttatgaggtacgaacatcacagaattactttgtaatttcagtttattgtaggcttggctttttggggagggtttacgtcttagacctcttagtgcttctttgtttcatggtgttctaacttcgaagcatctctgtagctttaatggattccttttctgaaagctttgctctctttcttccccctcggctttctcttaggcaagagggctaactgtaaagtaaggcttactgccttgtgtttccaaatgtgtccgaagaggaagtgtcttctgtgaatcctgttatgcatgaataacaggaaatagaaagaaattcactttcattattataaaagtaatatgttcgtttaaaaaattctaatgaagagctggagatgcaacccaggggtagagcacacactcagcatgcaggaggccctgggtccaatcttggaatctcctctcagttaacctgatctctagctgattagtagtgagtgcaagcccactttcctcttctgcctcattgctcagtgataacagctgttaaactttgtcttattctaaaactacctctgtgcaaatgctagcacaataatatatatcatatgcacatgattttttttttatcttgaaaagtaagtcagtatagctacaaagttcacttggcattgtcaacatttcacaggcgtaatattcctcctctagtactgtcctcttcattctttgtgaccaagtttggagagagtgcacaaatgccagggaggtttgtgggaaggtttctcatgttctggtaaggcgagtaagaaaatagtctcatgcaggtgaaatgagtgctatgcagtatatattataccagagaacagcaaatgaccaaattcacactgaactagttcagtaaaattggctttgtcaaagctttccttgcttaaaatgtaattccctgtcatcctagttctggtctggattcttttcctggagtcttgacttccagattccctgtggacttttgtttgagtttcaagcttttgaaatatagaaacctatctaacttaacaaacttgggagagaaaagactccagaacaactgaaaacagaccaggctaaatgaatagactttattcctctcttcttacctgcagttttcagatatgcagagttggagcggatcttagaggttgattcattcatgcctgaagaaaacacattttatagaccctgtgcccaagttcgtggtggacatcaccctttatttactaattgcactacataacaggcattttagaagactgctccagtcagagaccccgccttagaggaatctgtaaaccctgaactcctatcactcatgagcactagttatgtttggaatgccgtattaaaacaaaagttacatttctaaacttaaaattttctagcacagagacagtgggagtagctaactttgatagacatttttctactaaaagtctttctaagtacataatcttctgtaagttggaaaacagcaaaatagaacgtctcctacgtagttaatctttttgcataatttgcacatgtaggagttattagtatacgggtaagttttcactttttcccccaactggagtgtcttgtggctgggtttgaaaaagggaacgggaggccgctggaggggattggtaaatgagataaaacaccactcattcaactcagtgactcagcatttaaattttccataaaaggattaaaggaaaattaaacaaattcttaaagccaagactctggagaaacttgttggtgtgctttagttttcactgttatgactcatgaatttatgcataaattagtacatttataaaaacatagcctttttagagttttctgtttggctaaagtgccattgttagcatttggaattacctttttatgtcttatattttttccaaataaaaataaatgtttctgctgtcttactactgaaactacgttgtgagcactttaaatttctcaaagcagtttcgcctgttatacttggcgcttagtcatcgtcgtacacaacaggacctgattaagaaggctgtgctgcctctaagccgggctagattgtagccactagcaaccaggctgcaataatttccctttgatgacatcatccactgtggaagaacccagttgcttcagccagtcgaactatacagttccaacctcatcaaatatggcatctcccttgcctgctatagcagggggaggaaaaaatgccaccatctttttaatctagcaagcttctcttttcttcatctttttttttttcttttaaaaaaattctgatcatggatgcttcttccgatccctatttgccttatgacgggggaggagacaatatccccttgagggaattacataaaagaggtaagagcatccccttgctctgaatcctctgttggttgttgtgcatgcggctgggcggttctggggacaggctgtctgttgtcctcttgctgcaatgtgctgcttagttgccctgccttgttgctgtgggagaatgcgaccttcccagcagggctggccctccctgattgtttgctctgtgcagattagccctgcttcagatcacatagggctgcagactccatcttctgtgtgaaaatgctttcggtttgattgcagaaataagctgcctttacagccagctaaagtcctggtggttggttggcacctgcaaagtagtatttttgtacctctggaaacttatattttctttacacagcaatatcaagtgccggtatgccattctgttttggctgctgccaattaccatgtagactttgcaccacagagtaatagtaaaagctcctagctgcattttataacatttaaaaatagcaggaaagaagaattatttttgatttaacatgtttttgtcatttaacgtcttaactgattgacatactatattgtctgtctcgtgggtatcttgtacaacttgataggataaagcaatttagtttttttttttttttttaaatacatccagaatgtaagtcgtcagtagttttcgaacagataagtaatggtgttaatcttttggcaggctttgccttggtctccttaaagctaattaggtgttacttaattaaactgctcttttgctcattttcttaaattatttttttaaaagatagttggcatttgctgttctagaaataaacttcaagaaacattctttagccagatgacttcatgtatgagccatgttagtttgaattatttgcttggtgttataaactttatggtttaataccaacttttattatgtttacaaggtaaataaggaaaatttcaagtacattttgtatcctgagaacaaatttaagttccatagaatttaggaattacaatgtattcaacagatacttacttgtcatactgtgcctgcaaaacaataattagactctgaacaggtgcaacaattttctgtagaattagacaagtcttcttttggcaggtgttactaagtaggccatttcccaaggaacagggaatttgccaggcttttgtggtggagagaatagaatgaataaatgctgtggggagtaaagagcttgtcagaagatgattagttctgtggcaccaaaaccaagagatcagttttcctgtgagaagtaaaggaagcattgtagaaaaatagatgtgttgaagtctaccggtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattgtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatctacctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttccttgatgatgtcatacttatcctgtcccttttttttccacagctcgcggttgaggacaaactcttcgcggtctttccagtactcttggatcggaaacccgtcggcctccgaacggtactccgccaccgagggacctgagcgagtccgcatcgaccggatcggaaaacctcggatccgaattcatagataactgatccagtgcccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaatctagatccccctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagggggatcttcgcgatactgcatcgattagacaccgcggtggagctccagcttttgttccctttagtgagggttaattagttcttaatacgactcactatagggcgaattggctaccgggccgcccatcgagggtatcataagcttttaaatcgatagatgcgatcctgcaggtctccctatagtgagtcgtattaatttcgataagccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccggggtgcggaacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacctgcaggacgtcccaatgatcttaagttaa

SEQ ID NO: 25 - DNA, 570 bp, Artificial Sequence
  IRES-4
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 26 - DNA, 557 bp, Artificial Sequence
  IRES-5
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 27 - DNA, 550 bp, Artificial Sequence
  IRES-6
cccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacg

(19) United States
(12) Patent Application Publication
(10) Pub. No.: US 2022/0073945 A1
(43) Pub. Date: Mar. 10, 2022

(54) EXPRESSION VECTORS FOR EUKARYOTIC EXPRESSION SYSTEMS

(71) Applicant: Merck Sharp & Dohme Corp., Rahway, NJ (US)

(72) Inventors: Zhimei Du, Basking Ridge, NJ (US); Balrina Gupta, Basking Ridge, NJ (US); Steven C. Huhn, East Brunswick, NJ (US)

(73) Assignee: Merck Sharp & Dohme Corp., Rahway, NJ (US)

(21) Appl. No.: 17/416,286

(22) PCT Filed: Dec. 20, 2019
(86) PCT No.: PCT/US2019/067719

§ 371 Date: Jun. 18, 2021

Related U.S. Application Data
(60) Provisional application No. 62/783,343, filed on Dec. 21, 2018.

(51) Int. Cl. C12N 15/85; C12N 15/11; C12N 15/67

(52) U.S. Cl. CPC C12N 15/85; C12N 2800/90; C12N 15/67; C12N 15/11

(57) ABSTRACT

The invention provides expression vectors for expressing multi-chain recombinant proteins (e.g., biologics) in mammalian cells. Also provided are host cells comprising the expression vectors, methods of producing the multi-chain recombinant proteins, and methods of propagating the expression vectors.

### CROSS REFERENCE TO RELATED APPLICATIONS

This application claims the benefit of priority to U.S. Provisional Application No. 62/783,343, filed Dec. 21, 2018, the disclosure of which is incorporated by reference in its entirety.

### FIELD OF THE INVENTION

The present invention relates to expression vectors for expressing multi-chain recombinant proteins (e.g., biologics or vaccines) in eukaryotic cells.

### BACKGROUND OF THE INVENTION

There has been an increased interest in developing multi-chain protein therapeutics (e.g., monoclonal antibodies, bispecific antibodies, etc.). A bispecific antibody simultaneously binds to two different epitopes, which can be on the same antigen or different antigens. Bispecific antibodies can serve as mediators to direct immune effector cells (e.g., NK cells or T-cells) to target cells (e.g., tumor cells). They can also target two different receptors on the same cell to modulate multiple cell signaling pathways. Producing such complex bivalent molecules is quite challenging. Bispecific IgG molecules can be assembled from two different heavy chains and two different light chains expressed in the same producer cell. However, random assembly of the different heavy and light chains often results in a substantial number of nonfunctional impurity molecules, such as a homodimer of either heavy chain with its corresponding light chain, a heavy chain dimer, a light chain dimer, a half antibody with only one heavy chain and its corresponding light chain, a ¾ antibody that misses one light chain, or a heterodimer with wrong light chain association.

The existing methods that aim to solving the random assembly problem have not been able to achieve high yield bispecific antibody heterodimers with low impurities. Thus, there is still unmet need to design and develop a robust expression vector and system for producing high quantity and quality bispecific antibodies.

### SUMMARY OF THE INVENTION

The present disclosure provides expression vectors for expressing multi-chain recombinant proteins (e.g., bispecific antibodies) in eukaryotic expression systems. The disclosed expression vectors can 1) efficiently integrate into eukaryotic transcriptionally active hot spots; 2) block epigenetic gene silencing to ensure long term stable expression; 3) direct balanced expression of two genes of interest (GOI); 4) link the GOI and the eukaryotic selectable marker to ensure consistent expression of the GOI in the surviving eukaryotic cells; and 4) remain stable and generate consistent outcomes under various process conditions.

In one aspect, provided herein is an expression vector comprising:
(a) a first expression cassette comprising the following elements in the direction of 5′ to 3′: a first promoter operably linked to a first insertion site for a first GOI, an internal ribosome entry site (TRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) a DNA linker that connects the 5′ end of the first expression cassette and the 5′ end of the second expression cassette so that the first and the second expression cassettes are in the opposite directions;
(d) a third expression cassette comprising a second polynucleotide encoding a bacterial selectable marker; and
(e) a bacterial plasmid origin of replication.

In certain embodiments of the expression vector, the DNA linker is an insulator, a locus control region (LCR), a matrix attachment region (MAR), a scaffold attachment region (SAR), an expression augmenting sequence element (EASE), an adenovirus tripartite leader (TPL), or a ubiquitous chromatin opening element (UCOE). In one embodiment, the DNA linker is an insulator. In another embodiment, the DNA linker is a LCR. In yet another embodiment, the DNA linker is a MAR. In still another embodiment, the DNA linker is a SAR. In one embodiment, the DNA linker is an EASE. In another embodiment, the DNA linker is a TPL. In yet another embodiment, the DNA linker is a UCOE.

In some embodiments, the expression vector further comprises a first insulator at the 3′ end of the first expression cassette and a second insulator at the 3′ end of the second expression cassette.

In other embodiments, the expression vector further comprises two inverted terminal repeat (ITR) sequences flanking the portion of the expression vector comprising the first insulator, the first expression cassette, the DNA linker, the second expression cassette, and the second insulator.

Thus, in one embodiment, provided herein is an expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) an EASE that connects the 5′ end of the first expression cassette and the 5′ end of the second expression cassette so that the first and the second expression cassettes are in the opposite directions;
(d) a first insulator at the 3′ end of the first expression cassette and a second insulator at the 3′ end of the second expression cassette;
(e) a third expression cassette comprising a second polynucleotide encoding a bacterial selectable marker; and
(f) a bacterial plasmid origin of replication.

In another embodiment, provided herein is an expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) an EASE that connects the 5′ end of the first expression cassette and the 5′ end of the second expression cassette so that the first and the second expression cassettes are in the opposite directions;
(d) a first insulator at the 3′ end of the first expression cassette and a second insulator at the 3′ end of the second expression cassette;
(e) two inverted terminal repeat (ITR) sequences flanking the portion of the expression vector comprising (a)-(d);
(f) a third expression cassette comprising a second polynucleotide encoding a bacterial selectable marker; and
(g) a bacterial plasmid origin of replication.

In certain embodiments of the various expression vectors provided herein, the IRES comprises a polynucleotide sequence of SEQ ID NO:1, 2, 3, 25, 26, or 27. In one embodiment, the IRES comprises a polynucleotide sequence of SEQ ID NO:1. In another embodiment, the IRES comprises a polynucleotide sequence of SEQ ID NO:2. In yet another embodiment, the IRES comprises a polynucleotide sequence of SEQ ID NO:3. In one embodiment, the IRES comprises a polynucleotide sequence of SEQ ID NO:25. In another embodiment, the IRES comprises a polynucleotide sequence of SEQ ID NO:26. In yet another embodiment, the IRES comprises a polynucleotide sequence of SEQ ID NO:27.

In some embodiments of the various expression vectors provided herein, the eukaryotic selectable marker is a neomycin phosphotransferase, a histidinol dehydrogenase, a hygromycin B phosphotransferase, a xanthine-guanine phosphoribosyltransferase, a dihydrofolate reductase, a tryptophan synthetase, a puromycin N-acetyl-transferase, a thymidine kinase, an adenine phosphoribosyl transferase, a glutamine synthetase, an adenosine deaminase, or metallothionein-1. In one embodiment, the eukaryotic selectable marker is a neomycin phosphotransferase. In another embodiment, the eukaryotic selectable marker is a histidinol dehydrogenase. In yet another embodiment, the eukaryotic selectable marker is a hygromycin B phosphotransferase. In still another embodiment, the eukaryotic selectable marker is a xanthine-guanine phosphoribosyltransferase. In one embodiment, the eukaryotic selectable marker is a dihydrofolate reductase. In another embodiment, the eukaryotic selectable marker is a tryptophan synthetase. In yet another embodiment, the eukaryotic selectable marker is a puromycin N-acetyl-transferase. In still another embodiment, the eukaryotic selectable marker is a thymidine kinase. In one embodiment, the eukaryotic selectable marker is an adenine phosphoribosyl transferase. In another embodiment, the eukaryotic selectable marker is a glutamine synthetase. In yet another embodiment, the eukaryotic selectable marker is an adenosine deaminase. In still another embodiment, the eukaryotic selectable marker is metallothionein-1.

In certain embodiments of the various expression vectors provided herein, the first or the second promoter is a human cytomegalovirus (CMV) immediate-early promoter, a human elongation factor 1 alpha (EF1a) promoter, a SV40 promoter, a phosphoglycerate kinase 1 (PGK1) promoter, a human ubiquitin C (Ubc) promoter, a human β-actin promoter, a CAG promoter, a yeast transcription elongation factor 1 (TEF1) promoter, a yeast glyceraldehyde 3-phosphate dehydrogenase (GAPDH) promoter, or a yeast alcohol dehydrogenase 1 (ADH1) promoter. In one embodiment, the first or second promoter is a human CMV immediate-early promoter. In another embodiment, the first or second promoter is a human EFla promoter. In yet another embodiment, the first or second promoter is a SV40 promoter. In still another embodiment, the first or second promoter is a PGK1 promoter. In one embodiment, the first or second promoter is a human Ubc promoter. In another embodiment, the first or second promoter is a human β-actin promoter. In yet another embodiment, the first or second promoter is a CAG promoter. In still another embodiment, the first or second promoter is a yeast TEF1 promoter. In one embodiment, the first or second promoter is a yeast GAPDH promoter. In another embodiment, the first or second promoter is a yeast ADH1 promoter.

In some embodiments of the expression vector, the first and the second promoters are the same promoter. In one embodiment, the first and the second promoters are a human CMV immediate-early promoter.

In some embodiments of the expression vector, the first or the second expression cassette further comprises an enhancer. In one embodiment, the first expression cassette further comprises an enhancer. In another embodiment, the second expression cassette further comprises an enhancer. In yet another embodiment, the first expression cassettes further comprise a first enhancer, and the second expression cassettes further comprise a second enhancer. In one embodiment, the first and the second enhancers are the same.

In some embodiments of the various expression vectors provided herein, the enhancer is a human CMV immediate-early enhancer, a SV40 enhancer, a BK polyomarvirus (BKPyV) enhancer, an Epstein-Bar virus (EBV) enhancer, a c-Myc enhancer, an immunoglobulin heavy chain (IgH) enhancer, a Sp1-binding enhancer, an AP1-binding enhancer, or a CREB-binding enhancer. In one embodiment, the enhancer is a human CMV immediate-early enhancer. In another embodiment, the enhancer is a SV40 enhancer. In yet another embodiment, the enhancer is a BKPyV enhancer. In still another embodiment, the enhancer is an EBV enhancer. In one embodiment, the enhancer is a c-Myc enhancer. In another embodiment, the enhancer is an IgH enhancer. In yet another embodiment, the enhancer is a Sp1-binding enhancer. In still another embodiment, the enhancer is an AP1-binding enhancer. In one embodiment, the enhancer is a CREB-binding enhancer. In another embodiment, the first and the second enhancer are a human CMV immediate-early enhancer.

In other embodiments of the various expression vectors provided herein, the insulator is HMR tRNA^{Thr}, Chal UAS, UAS_{rpg}, STAR, scs, scs′, gypsy, Fab-7, Fab-8, fas^{wb}, sns, UR1, RO, Lys 5′ A, HS4, 3′HS, BEAD-1, HS2-6, DMD/ICR, 5′HSS, apoB (−57 kb), apoB (+43 kb), or DM1. In one embodiment, the insulator is HMR tRNA^{Thr}. In another embodiment, the insulator is Chal UAS. In yet another embodiment, the insulator is UAS_{rpg}. In still another embodiment, the insulator is STAR. In one embodiment, the insulator is scs. In another embodiment, the insulator is scs′. In yet another embodiment, the insulator is gypsy. In still another embodiment, the insulator is Fab-7. In one embodiment, the insulator is Fab-8. In another embodiment, the insulator is fas^{wb}. In yet another embodiment, the insulator is sns. In still another embodiment, the insulator is UR1. In one embodiment, the insulator is RO. In another embodiment, the insulator is Lys 5′ A. In yet another embodiment, the insulator is HS4. In still another embodiment, the insulator is 3′HS. In one embodiment, the insulator is BEAD-1. In another embodiment, the insulator is HS2-6. In yet another embodiment, the insulator is DMD/ICR. In still another embodiment, the insulator is 5′HS5. In one embodiment, the insulator is apoB (−57 kb). In another embodiment, the insulator is apoB (+43 kb). In yet another embodiment, the insulator is DM1. In certain embodiments of the expression vectors, the first and the second insulators are HS4.

In yet other embodiments of the various expression vectors provided herein, the ITR is Tc1 ITR, Tc3 ITR, Minos ITR, Mos1 ITR, Famar1 ITR, Osmar5 ITR, Fot1 ITR, Impala ITR, ISY100 ITR, Mboumar-9 ITR, Sleeping Beauty ITR, Himar1 ITR, Frog Prince ITR, Himar1 ITR, SB100X ITR, piggyBac ITR, or Tol1 ITR. In one embodiment, the ITR is Tc1 ITR. In another embodiment, the ITR is Tc3 ITR. In yet another embodiment, the ITR is Minos ITR. In still another embodiment, the ITR is Mos1 ITR. In one embodiment, the ITR is Famar1 ITR. In another embodiment, the ITR is Osmar5 ITR. In yet another embodiment, the ITR is Fot1 ITR. In still another embodiment, the ITR is Impala ITR. In one embodiment, the ITR is ISY100 ITR. In another embodiment, the ITR is Mboumar-9 ITR. In yet another embodiment, the ITR is Sleeping Beauty ITR. In still another embodiment, the ITR is Himar1 ITR. In one embodiment, the ITR is Frog Prince ITR. In another embodiment, the ITR is Hsmar1 ITR. In yet another embodiment, the ITR is SB100X ITR. In still another embodiment, the ITR is piggyBac ITR. In one embodiment, the ITR is Tol2 ITR.

In certain embodiments of the various expression vectors provided herein, the bacterial selectable marker is an ampicillin resistance gene, a tetracycline resistance gene, a hygromycin resistance gene, a kanamycin resistance gene, a blasticidin resistance gene, or the like. In one embodiment, the bacterial selectable marker is an ampicillin resistance gene. In another embodiment, the bacterial selectable marker is a tetracycline resistance gene. In yet another embodiment, the bacterial selectable marker is a hygromycin resistance gene. In still another embodiment, the bacterial selectable marker is a kanamycin resistance gene. In yet still another embodiment, the bacterial selectable marker is a blasticidin resistance gene.

In one embodiment, the expression vector comprises a polynucleotide sequence of SEQ ID NO:4. In another embodiment, the expression vector consists of a polynucleotide sequence of SEQ ID NO:4. In yet another embodiment, the expression vector consists essentially of a polynucleotide sequence of SEQ ID NO:4. In another specific embodiment, the expression vector comprises a polynucleotide sequence that is at least 60%, 70%, 80%, 90%, or 95% identical to the polynucleotide sequence of SEQ ID NO:4.

In one embodiment, the expression vector comprises a polynucleotide sequence of SEQ ID NO:5. In another embodiment, the expression vector consists of a polynucleotide sequence of SEQ ID NO:5. In yet another embodiment, the expression vector consists essentially of a polynucleotide sequence of SEQ ID NO:5. In another specific embodiment, the expression vector comprises a polynucleotide sequence that is at least 60%, 70%, 80%, 90%, or 95% identical to the polynucleotide sequence of SEQ ID NO:5.

In certain embodiments of various expression vectors provided herein, the first expression cassette further comprises the first GOI encoding a first polypeptide chain of a multi-chain recombinant protein.

In some embodiments of various expression vectors provided herein, the second expression cassette further comprises the second GOI encoding a second polypeptide chain of a multi-chain recombinant protein.

In other embodiments of various expression vectors provided herein, the first expression cassette further comprises the first GOI encoding a first polypeptide chain of a multi-chain recombinant protein, and the second expression cassette further comprises the second GOI encoding a second polypeptide chain of a multi-chain recombinant protein.

In one embodiment, the multi-chain recombinant protein is a therapeutic or prophylactic protein.

In some embodiments, the multi-chain recombinant protein is a monoclonal antibody. In one embodiment, the expression vector described herein comprises a first GOI encoding a heavy chain of a monoclonal antibody and a second GOI encoding a light chain of the monoclonal antibody.

In other embodiments, the multi-chain recombinant protein is a bispecific antibody. In one embodiment, the first polypeptide chain of the multi-chain recombinant protein is a first heavy chain of a bispecific antibody, and the second polypeptide chain of the multi-chain recombinant protein is a second heavy chain of the bispecific antibody. In another embodiment, the first polypeptide chain of the multi-chain recombinant protein is a first light chain of a bispecific antibody, and the second polypeptide chain of the multi-chain recombinant protein is a second light chain of the bispecific antibody. Thus, in one embodiment, the expression vector described herein comprises a first GOI encoding a first heavy chain of a bispecific antibody and a second GOI encoding a second heavy chain of the bispecific antibody. In another embodiment, the expression vector described herein comprises a first GOI encoding a first light chain of a bispecific antibody and a second GOI encoding a second light chain of the bispecific antibody.

In another aspect, provided is a host cell comprising various expression vectors disclosed herein.

In certain embodiments, the host cell is a mammalian host cell. In some embodiments, the host cell is a bacterial host cell. In other embodiments, the mammalian host cell is a CHO cell. In still other embodiments, the endogenous glutamine synthetase gene of the CHO cell is knocked out.

In some embodiments, the host cell comprises an expression vector described herein, wherein the first GOI encodes a heavy chain of a monoclonal antibody and the second GOI encodes a light chain of the monoclonal antibody.

In some embodiments, the host cell comprises a first expression vector and a second expression vector, wherein the first expression vector comprises a first GOI encoding a first heavy chain of a bispecific antibody and a second GOI encoding a second heavy chain of the bispecific antibody, wherein the second expression vector comprises a third GOI encoding a first light chain of the bispecific antibody and a fourth GOI encoding a second light chain of the bispecific antibody, and wherein the eukaryotic selectable marker of the first expression vector is different from the eukaryotic selectable marker of the second expression vector.

In yet another aspect, provided is a method of producing a multi-chain recombinant protein comprising a first polypeptide chain and a second polypeptide chain, comprising culturing the mammalian host cell disclosed herein under conditions in which the first polypeptide chain and the second polypeptide chain are expressed, and recovering the multi-chain recombinant protein comprising the first polypeptide chain and the second polypeptide chain from the culture, wherein the expression vector comprises a first GOI encoding the first polypeptide chain and a second GOI encoding the second polypeptide chain.

In one embodiment, provided is a method of producing a monoclonal antibody comprising a heavy chain and a light chain, comprising culturing the mammalian host cell disclosed herein under conditions in which the heavy chain and the light chain are expressed, and recovering the monoclonal antibody comprising the heavy chain and the light chain from the culture, wherein the expression vector comprises the a GOI encoding the heavy chain and a second GOI encoding the light chain.

In certain embodiments, provided is a method of producing a bispecific antibody, comprising culturing a host cell comprising a first expression vector and a second expression vector, wherein the first expression vector comprises a first GOI encoding a first heavy chain of the bispecific antibody and a second GOI encoding a second heavy chain of the bispecific antibody, wherein the second expression vector comprises a third GOI encoding a first light chain of the bispecific antibody and a fourth GOI encoding a second light chain of the bispecific antibody, under conditions in which the first heavy chain, the second heavy chain, the first light chain, and the second light chain of the bispecific antibody are expressed, and recovering the bispecific antibody from the culture.

In certain embodiments, provided is a method of producing a bispecific antibody, comprising culturing a host cell comprising a first expression vector and a second expression vector, wherein the first expression vector comprises a first GOI encoding a first heavy chain of the bispecific antibody and a second GOI encoding a second heavy chain of the bispecific antibody, wherein the second expression vector comprises a third GOI encoding a first light chain of the bispecific antibody and a fourth GOI encoding a second light chain of the bispecific antibody, wherein the eukaryotic selectable marker of the first expression vector is different from the eukaryotic selectable marker of the second expression vector, under conditions in which the first heavy chain, the second heavy chain, the first light chain, and the second light chain of the bispecific antibody are expressed, and recovering the bispecific antibody from the culture.

In still another aspect, provided is a method of propagating an expression vector, comprising culturing the bacterial host cell disclosed herein under conditions in which the expression vector is replicated, and recovering the expression vector from the culture.

### BRIEF DESCRIPTION OF THE DRAWINGS

FIGS. 1A and 1B illustrate a bispecific antibody heterodimer (FIG. 1A) and impurity species (FIG. 1B) in common bispecific antibody production.

FIGS. 2A-2C illustrate different vector designs for co-expression of two GOIs: (1) including an IRES (FIG. 2A), (2) including a Fu2A (FIG. 2B), or (3) including two independent expression cassettes in the same direction (FIG. 2C).

FIG. 3 illustrates an exemplary design of a bi-directional expression vector that can achieve balanced expression of two GOIs. P1 and p2 represent two promoters.

FIG. 4 illustrates an exemplary vector useful for a conventional 4-vector system for expressing a bispecific antibody, each vector encoding one chain of the bispecific antibody. The GOI is not shown in the figure.

FIGS. 5A and 5B illustrate exemplary vectors useful for an innovative bi-directional 2-vector system for expressing a bispecific antibody, each vector encoding two chains of the bispecific antibody. The difference between pCLD-BDDE-1 (FIG. 5A) and pCLD-BDDE-2 (FIG. 5B) is that pCLD-BDDE-1 includes two insulators whereas pCLD-BDDE-2 does not have any insulators. The GOIs are not shown in the figures.

FIGS. 6A-6D show that, compared to the conventional 4-vector system, the new bi-directional 2-vector system improves the balance among the four chains of the bispecific antibody, demonstrated at the DNA level (FIG. 6A), the mRNA level (FIG. 6B), the protein level (FIG. 6C), and the percentage of bispecific antibody heterodimer (FIG. 6D).

FIGS. 7A and 7B show the levels of the bispecific antibody heterodimer and the impurity species produced when using the new bi-directional 2-vector system (FIG. 7A) or the conventional 4-vector system (FIG. 7B).

FIG. 8 demonstrates that the new bi-directional 2-vector system achieves less clone-to-clone variation compared to the conventional 4-vector system.

FIG. 9 demonstrates that the new bi-directional 2-vector system achieves consistent product quality under different process conditions, whereas the conventional 4-vector system is highly sensitive to the process condition change.

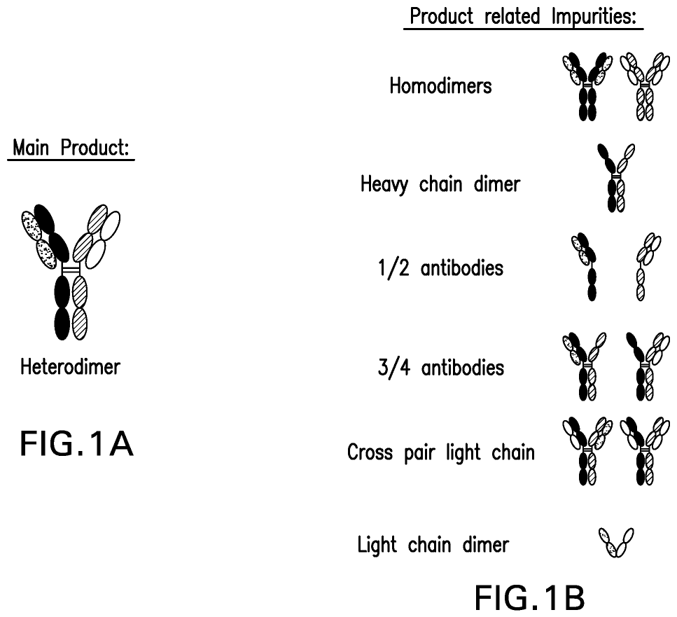

### DETAILED DESCRIPTION OF THE INVENTION

### I. General

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosure of such documents are incorporated herein by reference in their entirety for all purposes, and to the same extent as if each individual document was specifically and individually indicated to be incorporated by reference.

### II. Molecular Biology and Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

So that the invention may be more readily understood, certain technical and scientific terms are specifically defined below. Unless specifically defined elsewhere in this specification, all other technical and scientific terms use herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“About” when used to modify a numerically defined parameter, e.g., the length of a polynucleotide discussed herein, means that the parameter may vary by as much as 10% below or above the stated numerical value for that parameter. For example, a polynucleotide of about 100 bases may vary between 90 and 110 bases.

A “coding sequence” is a nucleotide sequence that encodes a biological product of interest (e.g., an RNA, polypeptide, protein, or enzyme) and when expressed, results in production of the product. A coding sequence is “under the control of,” “functionally associated with,” “operably linked to,” or “operably associated with” transcriptional or translational regulatory sequences in a cell when the regulatory sequences direct RNA polymerase-mediated transcription of the coding sequence into RNA, e.g., mRNA, which then may be trans-RNA spliced (if it contains introns) and, optionally, translated into a protein encoded by the coding sequence.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified composition.

“Express” and “expression” mean allowing or causing the information in a gene or coding sequence, e.g., an RNA or DNA, to become manifest; for example, producing a protein by activating the cellular functions involved in transcription and translation of a corresponding gene. A DNA sequence can be expressed in or by a cell to form an “expression product” such as an RNA (e.g., mRNA) or a protein. The expression product itself may also be said to be “expressed” by the cell.

“Expression vector” or “expression construct” means a vehicle (e.g., a plasmid) by which a polynucleotide comprising regulatory sequences operably linked to a coding sequence can be introduced into a host cell where the coding sequence is expressed using the transcription and translation machinery of the host cell.

“Expression cassette” means a double strand DNA polynucleotide that comprises elements sufficient to control expression of a gene, including but not limited to, a promoter operably linked to the gene sequence or operably linked to a multiple cloning site for inserting the gene sequence, and a polyA signal. In some embodiments, the expression cassette further comprises one or more regulatory elements that can regulate the expression of the gene at transcriptional, translational, and/or chromatin levels. “5′ end of an expression cassette” refers to the end of the expression cassette where the 5′ end of the coding strand is. “3′ end of an expression cassette” refers to the end of the expression cassette where the 3′ end of the coding strand is.

“DNA linker” means a fragment of DNA that locates between two other DNA fragments and connects the two other DNA fragments together to form a bigger DNA molecule. The DNA linker can be any polynucleotide sequence.

“Promoter” or “promoter sequence” is a segment of DNA that contains a regulatory region capable of recruiting an RNA polymerase (e.g., directly or through other promoter-bound proteins or substances) and initiating transcription of a coding sequence. Within the promoter sequence may be found a transcription initiation site (conveniently defined, for example, by mapping with nuclease Si), as well as protein binding domains (consensus sequences) responsible for the recruiting of RNA polymerase.

“Enhancer” or “enhancer sequence” is a DNA regulatory region that enhances transcription of a promoter independently of its distance, location, or orientation to the promoter. In certain embodiments, the enhancer is immediately adjacent to the promoter. In some embodiments, the enhancer is distant from the promoter. In other embodiments, the promoter and the enhancer are one combined sequence, referred as a “combo enhancer/promoter” herein.

“Internal ribosome entry site” or “IRES” is an RNA element or sequence that allows for translation initiation in a cap-independent manner by recruiting ribosomes directly. As used herein, the term “internal ribosome entry site” or “IRES” also encompasses the DNA sequence that can be transcribed into the RNA sequence that allows for translation initiation in a cap-independent manner by recruiting ribosomes directly.

“Regulatory element,” “regulatory region,” or “regulatory sequence,” as used herein, refers to a polynucleotide sequence that has the ability to regulate (such as, initiate, activate, enhance, increase, decrease, inhibit, suppress, or silence) expression of a gene. In some embodiments, the regulation is achieved by binding of cellular factors to the polynucleotide sequence. In other embodiments, the regulation is achieved by interaction between cellular factors. The regulation can occur at one or more different levels in the expression process from DNA to protein, including but not limited to transcriptional, translational, or chromatin levels.

“Insulator,” as used herein, refers to a class of DNA elements or sequences that possess an ability to isolate the proximal DNA region by preventing the positional effect from the surrounding chromosome area. In certain embodiments, the insulator can block enhancer when the insulator is situated between the enhancer and the promoter. In some embodiments, the insulator can act as barriers that prevent the advance of nearby condensed chromatin that might otherwise silence expression. In other embodiments, the insulator can block enhancer and act as barriers.

“Expression augmenting sequence element” or “EASE” is a DNA element or sequence that can increase expression of a protein when the DNA element or sequence is placed upstream of the promoter that controls the expression of the protein.

“Tripartite leader” or “TPL” is an RNA element or sequence in the 5′-untranslated region of adenovirus late-expressed mRNA that has an ability to initiate translation of the late-expressed mRNA in a cap-independent manner. As used herein, the term “tripartite leader” or “TPL” also encompasses the DNA sequence that can be transcribed into the RNA sequence in the 5′-untranslated region of adenovirus late-expressed mRNA that has an ability to initiate translation of the late-expressed mRNA in a cap-independent manner.

“Inverted terminal repeat” or “ITR,” in the context of transposon technology, refers to a DNA element or sequence and its inverted version at either end of a transposon that signals where the breakage and joining should occur.

“Selectable marker” or “selection marker” is a protein which allows the specific selection of cells that express this protein by the addition of a corresponding selecting agent to the culture medium. In certain embodiments, the selectable marker is a eukaryotic selectable marker, which allows selection of eukaryotic cells that express the marker protein. In some embodiments, the selectable marker is a bacterial selectable marker, which allows selection of bacterial cells that express the marker protein.

“Nucleic acid” or “polynucleotide” refers to a single- or double-stranded polymer of bases attached to a sugar phosphate backbone, and includes DNA and RNA molecules.

Each strand of DNA or RNA has a 5′ end and a 3′ end. “Direction,” as used herein, when referring to a DNA, means the 5′ to 3′ direction of the coding strand for a gene, and, when referring to an RNA, means the 5′ to 3′ direction of the RNA molecule. When two DNA or

RNA fragments are in the “same direction,” their 5′ to 3′ directions align and are in the same direction. When two DNA or RNA fragments are in the “opposite direction,” their 5′ to 3′ directions are opposite.

“Upstream” or “downstream,” as used herein, means relative positions of nucleic acid in DNA when referring to a gene or in RNA when referring to a gene transcript. When referring to the 5′ to 3′ direction in which RNA transcription takes place, upstream is toward the 5′ end of the RNA molecule and downstream is toward the 3′ end of the RNA. When referring to a double-stranded DNA, upstream is toward the 5′ end of the coding strand for the gene and downstream is toward the 3′ end of the coding strand. Some genes on the same DNA molecule may be transcribed in opposite directions, so the upstream and downstream areas of the molecule may change depending on which gene is used as the reference.

“Host cell” includes any cell of any organism that is used for the purpose of producing a recombinant protein encoded by an expression vector or propagating the expression vector introduced into the host cell. A “mammalian recombinant host cell” refers to a mammalian host cell that comprises a heterologous expression vector, which may or may not be integrated into the host cell chromosome. A “bacterial recombinant host cell” refers to a bacterial host cell that comprises a heterologous expression vector, which may or may not be integrated into the host cell chromosome.

“Monoclonal antibody” or “mAb,” as used herein, refers to a population of substantially homogeneous antibodies, i.e., the antibody molecules constituting the population are identical in amino acid sequence except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J Mol. Biol. 222: 581-597, for example. See also Presta (2005) J. Allergy Clin. Immunol. 116:731.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively.

“Bispecific antibody” means a monoclonal antibody or fragment thereof that can specifically bind to two different epitopes. The two different epitopes can be on the same antigen molecule or on two different antigen molecules. In some embodiments, the bispecific antibody comprises a first heavy chain and a first light chain that form a first specific binding site for a first antigen, and a second heavy chain and a second light chain that form a second specific binding site for a second antigen.

### III. Expression Vectors for Eukaryotic Expression System

This invention is related to expression vector design for expressing multi-chain recombinant proteins (e.g., monoclonal antibody or bispecific antibody) in eukaryotic cells.

Conventionally, bispecific antibodies are produced by cotransfecting a host cell with four expression vectors, each encoding a first heavy chain, a second heavy chain, a first light chain, or a second light chain of the bispecific antibody. Due to random assembly among the two heavy chains and the two light chains, a number of impurity species are usually produced along with the desired bivalent bispecific antibody. To improve the efficiency in producing bivalent bispecific antibodies, heterodimerization between the heavy chains was forced by introducing different mutations into the CH3 domain in each heavy chain, resulting in asymmetric antibodies. The mutations of knobs-and-hole design in the CH3 domain (Ridgway et al., Protein Eng., 1996, 9:617-621) and variations of similar approaches (Kreudenstein et al., MAbs, 2013, 5:646-654; Gunasekaran et al., JBC, 2010, 285:19637-19646) ensure formation of heterodimers between the two different heavy chains targeting different antigens. Even with heavy chain heterodimerization, random pairing of the two light chains with the two heavy chains is another critical issue for all these approaches. One solution is to use a common light chain that can enable binding to both antigens, but this may not be possible for all bispecific antibodies. Thus, mutations were also introduced into the CH1-CL as well as the VH-VL interface of the Fab fragments to ensure correct pairing of the light chains with the corresponding heavy chains (Spiess et al., Molecular Immunology, 2015, 67:95-106; Lewis et al., Nat. Biotech, 2014, 32:191-198).

Although the above various protein engineering approaches have significantly improved the efficiency in producing bispecific antibodies from mammalian cells, the production yield is much lower compared to that of monoclonal antibodies due to the impurity species, such as homodimers or half molecules. These impurities can have significant impacts on the safety, pharmacokinetics, and efficacy of bispecific antibody therapeutics. However, removing these product-related impurities can be challenging at the purification step and directly impact the manufacturing yield. Thus, it is imperative to generate a bispecific antibody product with a high percentage of heterodimers but minimal level of impurities directly from cell secretion.

The product-related impurities, such as homodimers and half molecules, result from imbalanced expression of individual heavy chain or light chain. Because balanced expression is not required in conventional monoclonal antibody expression, stable expression vectors specifically designed for expressing monoclonal antibodies cannot solve this problem. The current strategy across the industry is to significantly increase the effort during cloning and clone screening to identify the robust clone with the highest percentage of bispecific heterodimer and the lowest amount of impurity species. The caveat of this strategy is increased work load and lengthened timeline with unpredictable results. Usually, after screening of hundreds of clones, the percentage of heterodimers from a secreted product is only around 60-80%. In addition, since impurities are mainly due to imbalanced expression, the selected clone is highly sensitive to culture conditions that could affect protein production, such as host type, medium, temperature, scale, pH, etc. As a result, the selected clone using the conventional expression system may still not be stable, which can impede the following manufacturing development.

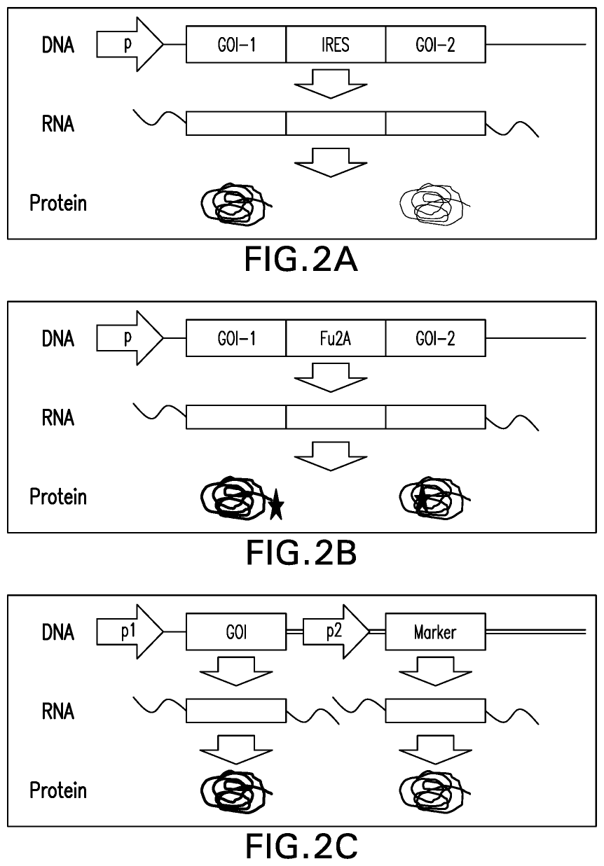

Designing an expression vector that allows consistent balanced co-expression of multiple GOIs simultaneously in CHO cells is critical. There are several known vector designs for co-expression of two GOIs: 1) using IRES to link two open reading frames (ORF) (FIG. 2A); 2) using Furin-2A systems to connect two GOIs through Furin and 2A consensus sites at the mRNA level (FIG. 2B), and the two proteins will be separated during translation and post-translational stage; 3) using two independent expression cassettes with the same or different promoters and polyA signals in the same direction (FIG. 2C). Each approach has its own limitations. For example, the IRES approach allows co-expression of two genes, but the translational efficiency of the upstream gene is much higher than that of the downstream gene, resulting in highly imbalanced level of the two proteins. Whereas the Furin-2A approach can express the two proteins at an equal level, it leaves overhanging residues at the C-terminal of the upstream protein or the N-terminal of the downstream protein, which could cause safety issues in patients. The two independent expression cassettes approach has been widely used for co-expression of two GOIs, but the expression efficiency of the two cassettes can be far from equal due to transcriptional interference or promoter suppression even when the two promoter strengths are similar (Kadesh et al., Mol. Cell. Biol., 1986, 6:2593-2601; Proudfoot, Nature, 1986, 322:562-565; Emerman, Cell, 1984, 39:459-467; Corbin et al., Nature, 1989, 337:279-282).

In this disclosure, an innovative bi-directional expression vector design and a 2-vector expression system are shown to improve balanced expression of two heavy chains and two light chains and increase the percentage of heterodimers from a secreted product to above 90% with minimal clone-to-clone or batch-to-batch variations.

Various DNA elements (e.g., an enhancer, a promoter, an insulator, an IRES, a LCR, a MAR, a SAR, an EASE, a TPL, a UCOE, or an ITR) can be selected and engineered into expression vectors.

Two ITR sequences can be engineered at both ends of the DNA fragment desired to be integrated into a host cell genome. Through appropriate vector design, the transposase that specifically recognizes the ITR sequences can remove the unfavorable bacteria-related element completely before integration of the desired DNA fragment into the mammalian genome to reduce epigenetic gene silencing. Furthermore, the transposon technology offers a large cargo-carrying capacity (up to 100-200 kb) which enables up to 10 times larger expression cassettes, compared to standard expression plasmid, to be easily integrated into a target genome.

IRES is a type of regulatory element that can be found in several viruses and cellular RNAs (reviewed in McBratney et. al. Current Opinion in Cell Biology 5:961, 1993). It is an RNA element that allows for translation initiation in a cap-independent manner by recruiting ribosomes directly. Therefore, inserting an IRES sequence between two ORFs allows co-expression of the two genes together in a bicistronic eukaryotic expression cassette (Kaufman R. J., et al., Nucleic Acids Res 19:4485, 1991). The upstream gene translation is initiated at the normal 5′ cap, whereas the downstream gene translation is initiated at the IRES element, thereby resulting in co-expression of two independent proteins from a single mRNA transcript. Since IRES-mediated ribosome recruitment ratio is relatively lower, genes encoding drug-resistance enzymes are usually placed downstream of IRES, serving as selection markers. In addition, designing IRES sequence variants to reduce expression level of downstream selection markers can further increase the expression level of the upstream GOI, which is highly desirable in biological applications. However, modulating IRES strength by designing IRES sequence variants has its limitation and is often unpredictable because the effect also depends on other regulatory elements in the expression vector. In addition, manipulating IRES alone can cause cell stress, and sometimes cells cannot be recovered well as high producers. Thus, IRES has not been uniformly utilized in all mammalian stable transfection. In this disclosure, an IRES variant is combined with other regulatory elements in vector design to achieve high expression of the GOI and appropriate expression level of the eukaryotic selection marker for stable cell selection.

Transcription of eukaryotic genes is one of the key steps in protein expression, and it is regulated by a variety of cis- and trans-acting regulatory elements (reviewed by Dillon and Grosveld, Trends Genet. 9:134; 1993). Two of the best characterized cis regulatory elements are promoters and enhancers, which recruit RNA polymerase II and transcriptional activators. However, merely a promoter and an enhancer are not sufficient to consistently maintain a high expression of the GOI due to epigenetic inhibitory effects. Epigenetic effects are stably heritable phenotypes resulting from changes in a chromosome without alterations in the DNA sequence (Berger S L, et al. 2009. Genes & Development. 23:781). Among all epigenetics-mediated gene repression mechanisms, heterochromatinization and position-effect are common pathways that result in gene repression. Cis regulatory elements regulating the chromatin structure and prevent heterochromatinization include but are not limited to LCR (Grosveld F., et al., Cell 51:975, 1987), MAR (Phi-Van et al., Mol Cell Biol 10:2302; 1980), SAR (Gasser and Laemmli, Trends Genet 3:16, 1987), insulator (Kellum and Schedl, Cell 64:941, 1991), and EASE (Aldrich et al., Cytotechnology 28: 9, 1998). These elements have been shown to support relatively higher expression of linked genes at distal chromatin sites, although the complete mechanism is not fully understood. One of the common features of these cis elements is their AT-rich sequences, suggesting the lower propensity for chromosome condensation in the local region, which allows for efficient chromosomal transcription activation and prevents position-effect mediated gene silencing. Thus, adding these epigenetic/chromosome level regulatory elements into expression vectors can decrease epigenetic gene silencing and improve long-term stability of selected clones.

Some other cis regulatory elements, such as adenovirus TPL, can enhance protein expression at translation initiation and post-transcriptional levels (Kaufman R. J. PNAS (1985) 82:689). TPL comprises three introns, which are critical to the translation of adenovirus late mRNA in a cap-independent manner. In addition, this element has been suggested in the regulation of mRNA stability and mRNA nuclear export, which also impacts protein expression level. Thus, inserting the TPL sequence downstream of the promoter can increase the efficiency of gene expression significantly at post-transcriptional levels in certain cell types with selected promoters, especially for long mRNA transcripts.

Any common DNA delivery approach known in the art, such as biological approach (e.g., virus-mediated), chemical approach (e.g., cationic polymer, calcium phosphate, or cationic lipid), or physical approach (e.g., direct injection, biolistic particle delivery, electroporation, laser-irradiation, sonoporation, or magnetic nanoparticle) can be used to achieve optimal efficiency of delivering the expression vectors disclosed herein into host cells.

In one aspect, provided herein is an expression vector comprising:
(a) a first expression cassette comprising the following elements in the direction of 5′ to 3′: a first promoter operably linked to a first insertion site for a first GOI, an IRES, a first polynucleotide encoding a eukaryotic selectable marker, and a first polyA signal;
(b) a second expression cassette comprising the following elements in the direction of 5′ to 3′: a second promoter operably linked to a second insertion site for a second GOI, and a second polyA signal;
(c) a DNA linker that connects the 5′ end of the first expression cassette and the 5′ end of the second expression cassette so that the first and the second expression cassettes are in the opposite directions;
(d) a third expression cassette comprising a second polynucleotide encoding a bacterial selectable marker; and
(e) a bacterial plasmid origin of replication.

The third expression cassettes may be arranged in the vector in any direction relative to the first and the second expression cassettes. In some embodiments, transcriptions of the third and the first expression cassettes are in the same direction. In other embodiments, transcriptions of the third and the second expression cassettes are in the same direction.

The insertion site typically comprises at least one restriction enzyme (RE) recognition sequence and may include two or more RE sequences to form a multiple cloning site.

In certain embodiments of the expression vector, the DNA linker is an insulator, a locus control region (LCR), a matrix attachment region (MAR), a scaffold attachment region (SAR), an expression augmenting sequence element (EASE), an adenovirus tripartite leader (TPL), or a ubiquitous chromatin opening element (UCOE). In one embodiment, the DNA linker is an insulator. In another embodiment, the DNA linker is a LCR. In yet another embodiment, the DNA linker is a MAR. In still another embodiment, the DNA linker is a SAR. In one embodiment, the DNA linker is an EASE. In another embodiment, the DNA linker is a TPL. In yet another embodiment, the DNA linker is a UCOE.

In some embodiments, the expression vector further comprises a first insulator at the 3′ end of the first expression cassette and a second insulator at the 3′ end of the second expression cassette. In some embodiments, the first insulator and the second insulators are the same insulator. In some embodiments, the first insulator and the second insulator are different insulators. In other embodiments, the first insulator and the second insulator are in the same direction. In yet other embodiments, the first insulator and the second insulator are in the opposite directions.

In other embodiments, the expression vector further comprises two inverted terminal repeat (ITR) sequences flanking the portion of the expression vector comprising the first insulator, the first expression cassette, the DNA linker, the second expression cassette, and the second insulator.

Thus, in one embodiment, provided herein is an expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) an EASE that connects the 5′ end of the first expression cassette and the 5′ end of the second expression cassette so that the first and the second expression cassettes are in the opposite directions;
(d) a first insulator at the 3′ end of the first expression cassette and a second insulator at the 3′ end of the second expression cassette;
(e) a third expression cassette comprising a second polynucleotide encoding a bacterial selectable marker; and
(f) a bacterial plasmid origin of replication.

In another embodiment, provided herein is an expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) an EASE that connects the 5′ end of the first expression cassette and the 5′ end of the second expression cassette so that the first and the second expression cassettes are in the opposite directions;
(d) a first insulator at the 3′ end of the first expression cassette and a second insulator at the 3′ end of the second expression cassette;
(e) two inverted terminal repeat (ITR) sequences flanking the portion of the expression vector comprising (a)-(d);
(f) a third expression cassette comprising a second polynucleotide encoding a bacterial selectable marker; and
(g) a bacterial plasmid origin of replication.

In certain embodiments of the various expression vectors provided herein, the IRES comprises a polynucleotide sequence of SEQ ID NO:1, 2, 3, 25, 26, or 27. In one embodiment, the IRES comprises a polynucleotide sequence of SEQ ID NO:1. In another embodiment, the IRES comprises a polynucleotide sequence of SEQ ID NO:2. In yet another embodiment, the IRES comprises a polynucleotide sequence of SEQ ID NO:3. In one embodiment, the IRES comprises a polynucleotide sequence of SEQ ID NO:25. In another embodiment, the IRES comprises a polynucleotide sequence of SEQ ID NO:26. In yet another embodiment, the IRES comprises a polynucleotide sequence of SEQ ID NO:27. In one embodiment, the IRES comprises a polynucleotide sequence that is about 90, 95, 96, 97, 98, or 99% identical to the polynucleotide sequence of SEQ ID NO:1. In another embodiment, the IRES comprises a polynucleotide sequence that is about 90, 95, 96, 97, 98, or 99% identical to the polynucleotide sequence of SEQ ID NO:2. In yet another embodiment, the IRES comprises a polynucleotide sequence that is about 90, 95, 96, 97, 98, or 99% identical to the polynucleotide sequence of SEQ ID NO:3. In one embodiment, the IRES comprises a polynucleotide sequence that is about 90, 95, 96, 97, 98, or 99% identical to the polynucleotide sequence of SEQ ID NO:25. In another embodiment, the IRES comprises a polynucleotide sequence that is about 90, 95, 96, 97, 98, or 99% identical to the polynucleotide sequence of SEQ ID NO:26. In yet another embodiment, the IRES comprises a polynucleotide sequence that is about 90, 95, 96, 97, 98, or 99% identical to the polynucleotide sequence of SEQ ID NO:27.

In some embodiments of the various expression vectors provided herein, the eukaryotic selectable marker is a neomycin phosphotransferase, a histidinol dehydrogenase, a hygromycin B phosphotransferase, a xanthine-guanine phosphoribosyltransferase, a dihydrofolate reductase, a tryptophan synthetase, a puromycin N-acetyl-transferase, a thymidine kinase, an adenine phosphoribosyl transferase, a glutamine synthetase, an adenosine deaminase, or metallothionein-1. In one embodiment, the eukaryotic selectable marker is a neomycin phosphotransferase. In another embodiment, the eukaryotic selectable marker is a histidinol dehydrogenase. In yet another embodiment, the eukaryotic selectable marker is a hygromycin B phosphotransferase. In still another embodiment, the eukaryotic selectable marker is a xanthine-guanine phosphoribosyltransferase. In one embodiment, the eukaryotic selectable marker is a dihydrofolate reductase. In another embodiment, the eukaryotic selectable marker is a tryptophan synthetase. In yet another embodiment, the eukaryotic selectable marker is a puromycin N-acetyl-transferase. In still another embodiment, the eukaryotic selectable marker is a thymidine kinase. In one embodiment, the eukaryotic selectable marker is an adenine phosphoribosyl transferase. In another embodiment, the eukaryotic selectable marker is a glutamine synthetase. In yet another embodiment, the eukaryotic selectable marker is an adenosine deaminase. In still another embodiment, the eukaryotic selectable marker is metallothionein-1.

In certain embodiments of the various expression vectors provided herein, the first or the second promoter is a human cytomegalovirus (CMV) immediate-early promoter, a human elongation factor 1 alpha (EF1a) promoter, a SV40 promoter, a phosphoglycerate kinase 1 (PGK1) promoter, a human ubiquitin C (Ubc) promoter, a human β-actin promoter, a CAG promoter, a yeast transcription elongation factor 1 (TEF1) promoter, a yeast glyceraldehyde 3-phosphate dehydrogenase (GAPDH) promoter, or a yeast alcohol dehydrogenase 1 (ADH1) promoter. In one embodiment, the first or second promoter is a human CMV immediate-early promoter. In another embodiment, the first or second promoter is a human EFla promoter. In yet another embodiment, the first or second promoter is a SV40 promoter. In still another embodiment, the first or second promoter is a PGK1 promoter. In one embodiment, the first or second promoter is a human Ubc promoter. In another embodiment, the first or second promoter is a human β-actin promoter. In yet another embodiment, the first or second promoter is a CAG promoter. In still another embodiment, the first or second promoter is a yeast TEF1 promoter. In one embodiment, the first or second promoter is a yeast GAPDH promoter. In another embodiment, the first or second promoter is a yeast ADH1 promoter.

In some embodiments of the expression vector, the first and the second promoters are the same promoter. In one embodiment, the first and the second promoters are a human CMV immediate-early promoter.

In certain embodiments of the various expression vectors provided herein, the first and/or the second expression cassette further comprise one or more regulatory elements. In some embodiments, the regulatory element is an enhancer, an insulator, a LCR, a MAR, a SAR, an EASE, a TPL, or a UCOE. In one embodiment, the regulatory element is an enhancer. In another embodiment, the regulatory element is an insulator. In yet another embodiment, the regulatory element is a LCR. In still another embodiment, the regulatory element is a MAR. In one embodiment, the regulatory element is a SAR. In another embodiment, the regulatory element is an EASE. In yet another embodiment, the regulatory element is a TPL. In still another embodiment, the regulatory element is a UCOE. In some embodiments, the first and/or the second expression cassette further comprise one regulatory element. In other embodiments, the first and/or the second expression cassette further comprise two regulatory elements. In yet other embodiments, the first and/or the second expression cassette further comprise three regulatory elements. In still other embodiments, the first and/or the second expression cassette further comprise four regulatory elements. In some embodiments, the first and/or the second expression cassette further comprise five regulatory elements. In other embodiments, the first and/or the second expression cassette further comprise six regulatory elements. In yet other embodiments, the first and/or the second expression cassette further comprise seven regulatory elements. In still other embodiments, the first and/or the second expression cassette further comprise eight or more regulatory elements.

In one embodiment, the first expression cassette further comprises an enhancer. In another embodiment, the second expression cassette further comprises an enhancer. In yet another embodiment, the first expression cassettes further comprise a first enhancer, and the second expression cassettes further comprise a second enhancer. In one embodiment, the first and the second enhancers are the same.

In some embodiments of the various expression vectors provided herein, the enhancer is a human CMV immediate-early enhancer, a SV40 enhancer, a BK polyomarvirus (BKPyV) enhancer, an Epstein-Bar virus (EBV) enhancer, a c-Myc enhancer, an immunoglobulin heavy chain (IgH) enhancer, a Sp1-binding enhancer, an AP1-binding enhancer, or a CREB-binding enhancer. In one embodiment, the enhancer is a human CMV immediate-early enhancer. In another embodiment, the enhancer is a SV40 enhancer. In yet another embodiment, the enhancer is a BKPyV enhancer. In still another embodiment, the enhancer is an EBV enhancer. In one embodiment, the enhancer is a c-Myc enhancer. In another embodiment, the enhancer is an IgH enhancer. In yet another embodiment, the enhancer is a Sp1-binding enhancer. In still another embodiment, the enhancer is an AP1-binding enhancer. In one embodiment, the enhancer is a CREB-binding enhancer. In another embodiment, the first and the second enhancer are a human CMV immediate-early enhancer.

In certain embodiments, the enhancer is immediately adjacent to the promoter. In some embodiments, the enhancer is distant from the promoter with other DNA fragments between the enhancer and the promoter. In other embodiments, the enhancer is upstream of the promoter. In yet other embodiments, the enhancer is downstream of the promoter. In still other embodiments, the enhancer and the promoter are combined together as a combo enhancer/promoter. In one specific embodiment, the combo enhancer/promoter is a human CMV immediate-early enhancer/promoter. In another specific embodiment, the combo enhancer/promoter is a synthetic CAG promoter that comprises a CMV immediate-early enhancer and a chicken β-actin promoter.

In other embodiments of the various expression vectors provided herein, the insulator is HMR tRNA^{Thr}, Chal UAS, UAS_{rpg}, STAR, scs, scs′, gypsy, Fab-7, Fab-8, fas^{wb}, sns, UR1, RO, Lys 5′ A, HS4, 3′HS, BEAD-1, HS2-6, DMD/ICR, 5′HS5, apoB (−57 kb), apoB (+43 kb), or DM1. In one embodiment, the insulator is HMR tRNA^{Thr}. In another embodiment, the insulator is Chal UAS. In yet another embodiment, the insulator is UAS_{rpg}. In still another embodiment, the insulator is STAR. In one embodiment, the insulator is scs. In another embodiment, the insulator is scs′. In yet another embodiment, the insulator is gypsy. In still another embodiment, the insulator is Fab-7. In one embodiment, the insulator is Fab-8. In another embodiment, the insulator is fas^{wb}. In yet another embodiment, the insulator is sns. In still another embodiment, the insulator is UR1. In one embodiment, the insulator is RO. In another embodiment, the insulator is Lys 5′ A. In yet another embodiment, the insulator is HS4. In still another embodiment, the insulator is 3′HS. In one embodiment, the insulator is BEAD-1. In another embodiment, the insulator is HS2-6. In yet another embodiment, the insulator is DMD/ICR. In still another embodiment, the insulator is 5′HS5. In one embodiment, the insulator is apoB (−57 kb). In another embodiment, the insulator is apoB (+43 kb). In yet another embodiment, the insulator is DM1. In certain embodiments of the expression vectors, the first and the second insulators are HS4.

In yet other embodiments of the various expression vectors provided herein, the ITR is Tc1 ITR, Tc3 ITR, Minos ITR, Mos1 ITR, Famar1 ITR, Osmar5 ITR, Fot1 ITR, Impala ITR, ISY 100 ITR, Mboumar-9 ITR, Sleeping Beauty ITR, Himar1 ITR, Frog Prince ITR, Hsmar1 ITR, SB100X ITR, piggyBac ITR, or Tol1 ITR. In one embodiment, the ITR is Tc1 ITR. In another embodiment, the ITR is Tc3 ITR. In yet another embodiment, the ITR is Minos ITR. In still another embodiment, the ITR is Mos/ITR. In one embodiment, the ITR is Famar1 ITR. In another embodiment, the ITR is Osmar5 ITR. In yet another embodiment, the ITR is Fot1 ITR. In still another embodiment, the ITR is Impala ITR. In one embodiment, the ITR is ISY 100 ITR. In another embodiment, the ITR is Mboumar-9 ITR. In yet another embodiment, the ITR is Sleeping Beauty ITR. In still another embodiment, the ITR is Himar1 ITR. In one embodiment, the ITR is Frog Prince ITR. In another embodiment, the ITR is Hsmar1 ITR. In yet another embodiment, the ITR is SB100X ITR. In still another embodiment, the ITR is piggyBac ITR. In one embodiment, the ITR is Tol1 ITR.

In certain embodiments of the various expression vectors provided herein, the bacterial selectable marker is an ampicillin resistance gene, a tetracycline resistance gene, a hygromycin resistance gene, a kanamycin resistance gene, a blasticidin resistance gene, or the like. In one embodiment, the bacterial selectable marker is an ampicillin resistance gene. In another embodiment, the bacterial selectable marker is a tetracycline resistance gene. In yet another embodiment, the bacterial selectable marker is a hygromycin resistance gene. In still another embodiment, the bacterial selectable marker is a kanamycin resistance gene. In yet still another embodiment, the bacterial selectable marker is a blasticidin resistance gene.

In still other embodiments of the various expression vectors provided herein, the polyA signal is a thymidine kinase gene polyA signal, a SV40 early gene polyA signal, a SV40 late gene polyA signal, a β-globin gene polyA signal, or the like. In some embodiments, the polyA signal is a thymidine kinase gene polyA signal. In certain embodiments, the polyA signal is a SV40 early gene polyA signal. In other embodiments, the polyA signal is a SV40 late gene polyA signal. In yet other embodiments, the polyA signal is a β-globin gene polyA signal.

A bacterial plasmid origin of replication is also present in various expression vectors disclosed herein to facilitate preparation of large quantities of the vector in bacteria cells. Non-limiting examples of plasmid replication origins include pUC origins derived from pBR322.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:1.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:1.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:1.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:1.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:1.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:1.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:1.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:1.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:1.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:1.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:1.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:1.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:2.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:2.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:2.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:2.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:2.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:2.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:2.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:2.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:2.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:2.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:2.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:2.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:3.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:3.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:3.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:3.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:3.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:3.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:3.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:3.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:3.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:3.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:3.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:3.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:25.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:25.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:25.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:25.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:25.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:25.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:25.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:25.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:25.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:25.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:25.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:25.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:26.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:26.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:26.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:26.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:26.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:26.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:26.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:26.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:26.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:26.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:26.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:26.

In one embodiment of various expression vectors provided herein, the first insulator and the second insulator are HS4, the first and the second promoters are a human CMV immediate-early promoter, the DNA linker is an EASE, the eukaryotic selectable marker is a glutamine synthetase, and the IRES comprises a polynucleotide sequence of SEQ ID NO:27.

In another embodiment of various expression vectors provided herein, the first insulator and the second insulator are HS4, the first and the second promoters are a human CMV immediate-early promoter, the DNA linker is an EASE, the eukaryotic selectable marker is a neomycin phosphotransferase, and the IRES comprises a polynucleotide sequence of SEQ ID NO:27.

In yet another embodiment of various expression vectors provided herein, the first insulator and the second insulator are HS4, the first and the second promoters are a human CMV immediate-early promoter, the DNA linker is an EASE, the eukaryotic selectable marker is a puromycin N-acetyl-transferase, and the IRES comprises a polynucleotide sequence of SEQ ID NO:27.

In one embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a glutamine synthetase, and the IRES comprises a polynucleotide sequence of SEQ ID NO:27.

In another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a neomycin phosphotransferase, and the IRES comprises a polynucleotide sequence of SEQ ID NO:27.

In yet another embodiment of various expression vectors provided herein, the first insulator and the second insulator are HS4, the first and the second promoters are a human CMV immediate-early promoter, the DNA linker is an EASE, the ITR is piggyBac ITR, the eukaryotic selectable marker is a puromycin N-acetyl-transferase, and the IRES comprises a polynucleotide sequence of SEQ ID NO:27.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a glutamine synthetase, and the IRES comprises a polynucleotide sequence of SEQ ID NO:27.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a neomycin phosphotransferase, and the IRES comprises a polynucleotide sequence of SEQ ID NO:27.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the eukaryotic selectable marker is a puromycin N-acetyl-transferase, and the IRES comprises a polynucleotide sequence of SEQ ID NO:27.

In one embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a glutamine synthetase, and the IRES comprises a polynucleotide sequence of SEQ ID NO:27.

In another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a neomycin phosphotransferase, and the IRES comprises a polynucleotide sequence of SEQ ID NO:27.

In yet another embodiment of various expression vectors provided herein, the first insulator and the second insulator are HS4, the first enhancer and the first promoter are a first combo enhancer/promoter, the second enhancer and the second promoter are a second combo enhancer/promoter, wherein the first and the second combo enhancer/promoters are a human CMV immediate-early enhancer/promoter, the DNA linker is an EASE, the ITR is piggyBac ITR, the eukaryotic selectable marker is a puromycin N-acetyl-transferase, and the IRES comprises a polynucleotide sequence of SEQ ID NO:27.

In some embodiments, the IRES comprises a polynucleotide sequence that is at least 95%, 96%, 97%, 98%, or 99% identical to the polynucleotide sequence of SEQ ID NO:1. In some embodiments, the IRES comprises a polynucleotide sequence that is at least 95%, 96%, 97%, 98%, or 99% identical to the polynucleotide sequence of SEQ ID NO:2. In some embodiments, the IRES comprises a polynucleotide sequence that is at least 95%, 96%, 97%, 98%, or 99% identical to the polynucleotide sequence of SEQ ID NO:3. In some embodiments, the IRES comprises a polynucleotide sequence that is at least 95%, 96%, 97%, 98%, or 99% identical to the polynucleotide sequence of SEQ ID NO:25. In some embodiments, the IRES comprises a polynucleotide sequence that is at least 95%, 96%, 97%, 98%, or 99% identical to the polynucleotide sequence of SEQ ID NO:26. In some embodiments, the IRES comprises a polynucleotide sequence that is at least 95%, 96%, 97%, 98%, or 99% identical to the polynucleotide sequence of SEQ ID NO:27.

In one embodiment, the expression vector comprises a polynucleotide sequence of SEQ ID NO:4. In another embodiment, the expression vector consists of a polynucleotide sequence of SEQ ID NO:4. In yet another embodiment, the expression vector consists essentially of a polynucleotide sequence of SEQ ID NO:4. In another specific embodiment, the expression vector comprises a polynucleotide sequence that is at least 60%, 70%, 80%, 90%, or 95% identical to the polynucleotide sequence of SEQ ID NO:4.

In one embodiment, the expression vector comprises a polynucleotide sequence of SEQ ID NO:5. In another embodiment, the expression vector consists of a polynucleotide sequence of SEQ ID NO:5. In yet another embodiment, the expression vector consists essentially of a polynucleotide sequence of SEQ ID NO:5. In another specific embodiment, the expression vector comprises a polynucleotide sequence that is at least 60%, 70%, 80%, 90%, or 95% identical to the polynucleotide sequence of SEQ ID NO:5.

In one specific embodiment, the 5′ piggyBac ITR comprises a polynucleotide sequence that is at least 95%, 96%, 97%, 98%, or 99% identical to the polynucleotide sequence of SEQ ID NO:6, and the 3′ piggyBac ITR comprises a polynucleotide sequence that is at least 95%, 96%, 97%, 98%, or 99% identical to the polynucleotide sequence of SEQ ID NO:7. In another embodiment, the 5′ piggyBac ITR comprises the polynucleotide sequence of SEQ ID NO:6, and the 3′ piggyBac ITR comprises the polynucleotide sequence of SEQ ID NO:7. In yet another embodiment, the 5′ piggyBac ITR consists of the polynucleotide sequence of SEQ ID NO:6, and the 3′ piggyBac ITR consists of the polynucleotide sequence of SEQ ID NO:7.

In one specific embodiment, the HS4 insulator comprises a polynucleotide sequence that is at least 95%, 96%, 97%, 98%, or 99% identical to the polynucleotide sequence of SEQ ID NO:8. In another embodiment, the HS4 insulator comprises the polynucleotide sequence of SEQ ID NO:8. In yet another embodiment, the HS4 insulator consists of the polynucleotide sequence of SEQ ID NO:8.

In one specific embodiment, the EASE comprises a polynucleotide sequence that is at least 95%, 96%, 97%, 98%, or 99% identical to the polynucleotide sequence of SEQ ID NO:9. In another embodiment, the EASE comprises the polynucleotide sequence of SEQ ID NO:9. In yet another embodiment, the EASE consists of the polynucleotide sequence of SEQ ID NO:9.

In one specific embodiment, the β-globin gene polyA signal comprises a polynucleotide sequence that is at least 95%, 96%, 97%, 98%, or 99% identical to the polynucleotide sequence of SEQ ID NO:10. In another embodiment, the β-globin gene polyA signal comprises the polynucleotide sequence of SEQ ID NO:10. In yet another embodiment, the β-globin gene polyA signal consists of the polynucleotide sequence of SEQ ID NO:10.

In one specific embodiment, the human CMV immediate-early enhancer/promoter comprises a polynucleotide sequence that is at least 95%, 96%, 97%, 98%, or 99% identical to the polynucleotide sequence of SEQ ID NO:11. In another embodiment, the human CMV immediate-early enhancer/promoter comprises the polynucleotide sequence of SEQ ID NO:11. In yet another embodiment, the human CMV immediate-early enhancer/promoter consists of the polynucleotide sequence of SEQ ID NO:11.

In one specific embodiment, the TPL comprises a polynucleotide sequence that is at least 95%, 96%, 97%, 98%, or 99% identical to the polynucleotide sequence of SEQ ID NO:12. In another embodiment, the TPL comprises the polynucleotide sequence of SEQ ID NO:12. In yet another embodiment, the TPL consists of the polynucleotide sequence of SEQ ID NO:12.

In one specific embodiment, the bacterial plasmid origin of replication comprises a polynucleotide sequence that is at least 95%, 96%, 97%, 98%, or 99% identical to the polynucleotide sequence of SEQ ID NO:13. In another embodiment, the bacterial plasmid origin of replication comprises the polynucleotide sequence of SEQ ID NO:13. In yet another embodiment, the bacterial plasmid origin of replication consists of the polynucleotide sequence of SEQ ID NO:13.

In one specific embodiment, the ampicillin resistance gene comprises a polynucleotide sequence that is at least 95%, 96%, 97%, 98%, or 99% identical to the polynucleotide sequence of SEQ ID NO:14. In another embodiment, the ampicillin resistance gene comprises the polynucleotide sequence of SEQ ID NO:14. In yet another embodiment, the ampicillin resistance gene consists of the polynucleotide sequence of SEQ ID NO:14.

In one specific embodiment, the gene encoding the glutamine synthetase comprises a polynucleotide sequence that is at least 95%, 96%, 97%, 98%, or 99% identical to the polynucleotide sequence of SEQ ID NO:15. In another embodiment, the gene encoding the glutamine synthetase comprises the polynucleotide sequence of SEQ ID NO:15. In yet another embodiment, the gene encoding the glutamine synthetase consists of the polynucleotide sequence of SEQ ID NO:15.

In one specific embodiment, the gene encoding the neomycin phosphotransferase comprises a polynucleotide sequence that is at least 95%, 96%, 97%, 98%, or 99% identical to the polynucleotide sequence of SEQ ID NO:16. In another embodiment, the gene encoding the neomycin phosphotransferase comprises the polynucleotide sequence of SEQ ID NO:16. In yet another embodiment, the gene encoding the neomycin phosphotransferase consists of the polynucleotide sequence of SEQ ID NO:16.

In one specific embodiment, the gene encoding the puromycin N-acetyl-transferase comprises a polynucleotide sequence that is at least 95%, 96%, 97%, 98%, or 99% identical to the polynucleotide sequence of SEQ ID NO:17. In another embodiment, the gene encoding the puromycin N-acetyl-transferase comprises the polynucleotide sequence of SEQ ID NO:17. In yet another embodiment, the gene encoding the puromycin N-acetyl-transferase consists of the polynucleotide sequence of SEQ ID NO:17.

In one specific embodiment, the blasticidin resistance gene comprises a polynucleotide sequence that is at least 95%, 96%, 97%, 98%, or 99% identical to the polynucleotide sequence of SEQ ID NO:18. In another embodiment, the blasticidin resistance gene comprises the polynucleotide sequence of SEQ ID NO:18. In yet another embodiment, the blasticidin resistance gene consists of the polynucleotide sequence of SEQ ID NO:18.

In one specific embodiment, the SV40 late polyA signal comprises a polynucleotide sequence that is at least 95%, 96%, 97%, 98%, or 99% identical to the polynucleotide sequence of SEQ ID NO:19. In another embodiment, the SV40 late polyA signal comprises the polynucleotide sequence of SEQ ID NO:19. In yet another embodiment, the SV40 late polyA signal consists of the polynucleotide sequence of SEQ ID NO:19.

In one specific embodiment, the SV40 promoter comprises a polynucleotide sequence that is at least 95%, 96%, 97%, 98%, or 99% identical to the polynucleotide sequence of SEQ ID NO:20. In another embodiment, the SV40 promoter comprises the polynucleotide sequence of SEQ ID NO:20. In yet another embodiment, the SV40 promoter consists of the polynucleotide sequence of SEQ ID NO:20.

In one specific embodiment, the SV40 enhancer comprises a polynucleotide sequence that is at least 95%, 96%, 97%, 98%, or 99% identical to the polynucleotide sequence of SEQ ID NO:21. In another embodiment, the SV40 enhancer comprises the polynucleotide sequence of SEQ ID NO:21. In yet another embodiment, the SV40 enhancer consists of the polynucleotide sequence of SEQ ID NO:21.

Polypeptides that can be encoded by the GOI and expressed by various expression vectors described herein include, but are not limited to, therapeutic polypeptides such as adhesion molecules, antibody light and/or heavy chains, cytokines, enzymes, lymphokines, and receptors, etc.

In certain embodiments of various expression vectors provided herein, the first expression cassette further comprises the first GOI encoding a first polypeptide chain of a multi-chain recombinant protein.

In some embodiments of various expression vectors provided herein, the second expression cassette further comprises the second GOI encoding a second polypeptide chain of a multi-chain recombinant protein.

In other embodiments of various expression vectors provided herein, the first expression cassette further comprises the first GOI encoding a first polypeptide chain of a multi-chain recombinant protein, and the second expression cassette further comprises the second GOI encoding a second polypeptide chain of the multi-chain recombinant protein.

In one embodiment, the multi-chain recombinant protein is a therapeutic or prophylactic protein.

In some embodiments, the multi-chain recombinant protein is a monoclonal antibody. In one embodiment, the expression vector described herein comprises a first GOI encoding a heavy chain of a monoclonal antibody and a second GOI encoding a light chain of the monoclonal antibody.

In other embodiments, the multi-chain recombinant protein is a bispecific antibody. In one embodiment, the first polypeptide chain of the multi-chain recombinant protein is a first heavy chain of a bispecific antibody, and the second polypeptide chain of the multi-chain recombinant protein is a second heavy chain of the bispecific antibody. In another embodiment, the first polypeptide chain of the multi-chain recombinant protein is a first light chain of a bispecific antibody, and the second polypeptide chain of the multi-chain recombinant protein is a second light chain of the bispecific antibody. Thus, in one embodiment, the expression vector described herein comprises a first GOI encoding a first heavy chain of a bispecific antibody and a second GOI encoding a second heavy chain of the bispecific antibody. In another embodiment, the expression vector described herein comprises a first GOI encoding a first light chain of a bispecific antibody and a second GOI encoding a second light chain of the bispecific antibody.

In another aspect, provided is a host cell comprising various expression vectors disclosed herein.

In certain embodiments, the host cell is a mammalian host cell. In some embodiments, the host cell is a bacterial host cell.

Suitable mammalian host cells include but are not limited to hamster cells, such as CHO, CHO-K1, CHO-DUKX, CHO-DUKX B1, CHO-DG44, CHO-DBX11, CHOK1SV™, HD-BIOP1, CHOZN®, BHK21, BHK TK⁻, or ExpiCHO, as well as derivatives/descendants of these hamster cell lines. Also suitable are myeloma cells from the mouse, such as NSO or Sp2/0-AG14 cells, and human cell lines, such as HEK293, Hela, Jerkat, TP1, or PER.C6, as well as derivatives/descendants of these mouse and human cell lines.

In certain embodiments of various mammalian recombinant host cells provided herein, the mammalian host cell is a CHO cell. In one embodiment, the endogenous glutamine synthetase gene of the CHO cell is knocked out. In another embodiment, the mammalian host cell is a CHOK1SV™ cell. In yet another embodiment, the mammalian host cell is a HD-BIOP1 cell. In still another embodiment, the mammalian host cell is a CHOZN® cell.

Suitable bacterial host cells include but are not limited to the bacterial host cells that are commonly used for molecular cloning, transformation, and/or propagation of expression vectors by an ordinary person in the art, for example, DH5α™, DH10B™, JM109, TOP10, etc., as well as derivatives and modifications of them.

In some embodiments, the host cell comprises an expression vector described herein, wherein the first GOI encodes a first polypeptide chain of a multi-chain recombinant protein and the second GOI encodes a second polypeptide chain of the multi-chain recombinant protein.

In some embodiments, the host cell comprises an expression vector described herein, wherein the first GOI encodes a heavy chain of a monoclonal antibody and the second GOI encodes a light chain of the monoclonal antibody.

In some embodiments, the host cell comprises a first expression vector and a second expression vector, wherein the first expression vector comprises a first GOI encoding a first polypeptide chain of a multi-chain recombinant protein and a second GOI encoding a second polypeptide chain of the multi-chain recombinant protein, wherein the second expression vector comprises a third GOI encoding a third polypeptide chain of the multi-chain recombinant protein and a fourth GOI encoding a fourth polypeptide chain of the multi-chain recombinant protein, and wherein the eukaryotic selectable marker of the first expression vector is different from the eukaryotic selectable marker of the second expression vector.

In some embodiments, the host cell comprises a first expression vector and a second expression vector, wherein the first expression vector comprises a first GOI encoding a first heavy chain of a bispecific antibody and a second GOI encoding a second heavy chain of the bispecific antibody, wherein the second expression vector comprises a third GOI encoding a first light chain of the bispecific antibody and a fourth GOI encoding a second light chain of the bispecific antibody, and wherein the eukaryotic selectable marker of the first expression vector is different from the eukaryotic selectable marker of the second expression vector.

In yet another aspect, provided is a method of producing a multi-chain recombinant protein comprising a first polypeptide chain and a second polypeptide chain, comprising culturing the mammalian host cell disclosed herein under conditions in which the first polypeptide chain and the second polypeptide chain are expressed, and recovering the multi-chain recombinant protein comprising the first polypeptide chain and the second polypeptide chain from the culture, wherein the expression vector comprises a first GOI encoding the first polypeptide chain and a second GOI encoding the second polypeptide chain.

In one embodiment, provided is a method of producing a monoclonal antibody comprising a heavy chain and a light chain, comprising culturing the mammalian host cell disclosed herein under conditions in which the heavy chain and the light chain are expressed, and recovering the monoclonal antibody comprising the heavy chain and the light chain from the culture, wherein the expression vector comprises a first GOI encoding the heavy chain and a second GOI encoding the light chain.

In certain embodiments, provided is a method of producing a multi-chain recombinant protein comprising four polypeptide chains, comprising culturing a host cell comprising a first expression vector and a second expression vector, wherein the first expression vector comprises a first GOI encoding a first polypeptide chain of the multi-chain recombinant protein and a second GOI encoding a second polypeptide chain of the multi-chain recombinant protein, wherein the second expression vector comprises a third GOI encoding a third polypeptide chain of the multi-chain recombinant protein and a fourth GOI encoding a fourth polypeptide chain of the multi-chain recombinant protein, wherein the eukaryotic selectable marker of the first expression vector is different from the eukaryotic selectable marker of the second expression vector, under conditions in which the first polypeptide chain, the second polypeptide chain, the third polypeptide chain, and the fourth polypeptide chain of the multi-chain recombinant protein are expressed, and recovering the multi-chain recombinant protein from the culture.

In certain embodiments, provided is a method of producing a bispecific antibody, comprising culturing a host cell comprising a first expression vector and a second expression vector, wherein the first expression vector comprises a first GOI encoding a first heavy chain of the bispecific antibody and a second GOI encoding a second heavy chain of the bispecific antibody, wherein the second expression vector comprises a third GOI encoding a first light chain of the bispecific antibody and a fourth GOI encoding a second light chain of the bispecific antibody, wherein the eukaryotic selectable marker of the first expression vector is different from the eukaryotic selectable marker of the second expression vector, under conditions in which the first heavy chain, the second heavy chain, the first light chain, and the second light chain of the bispecific antibody are expressed, and recovering the bispecific antibody from the culture.

In still another aspect, provided is a method of propagating an expression vector, comprising culturing the bacterial host cell disclosed herein under conditions in which the expression vector is replicated, and recovering the expression vector from the culture.

In any embodiments of various expression vectors, host cells, methods of propagating expression vectors, methods of producing polypeptides encoded by the GOI, or methods of producing monoclonal antibodies, the polynucleotide sequence for an individual vector element or component (e.g., an enhancer, a promoter, an IRES, an insulator, a LCR, a MAR, a SAR, an EASE, a TPL, or an ITR) can be obtained from a different species than the species from which the sequences disclosed herein are obtained. For example, a species variant of a human β-globin polyA signal, such as a mouse or hamster β-globin polyA signal, can be used in the expression vectors. Similarly, a species variant of an adenovirus TPL, such as a human adenovirus B TPL, a human adenovirus C TPL, a human adenovirus E TPL, or an ovine adenovirus TPL, can be used in the expression vectors.

### EXAMPLES

These examples are intended to further clarify the present invention and not to limit the invention. Any composition or method, in whole or in part, set forth in the examples form a part of the present invention.

### Example 1: Construction of Expression Vectors

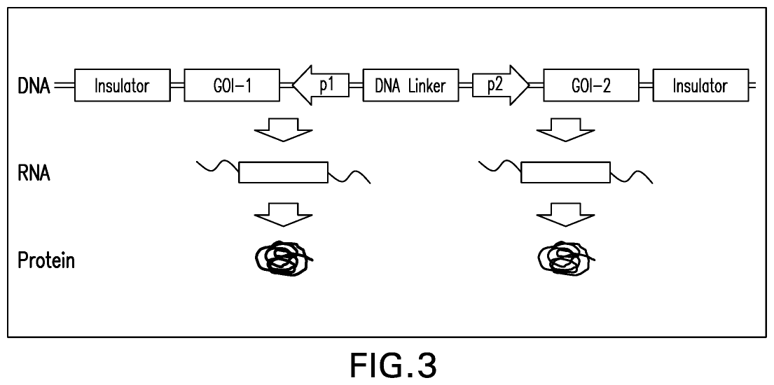

FIG. 3 illustrates an exemplary bi-directional expression vector comprising a first expression cassette and a second expression cassette connected by a DNA linker, wherein the first expression cassette and the second expression cassette are in the opposite direction. In FIG. 3, p1 and p2 represent the first promoter and the second promoter, respectively.

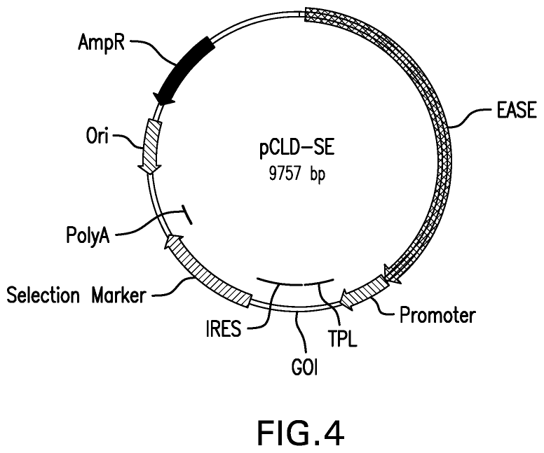

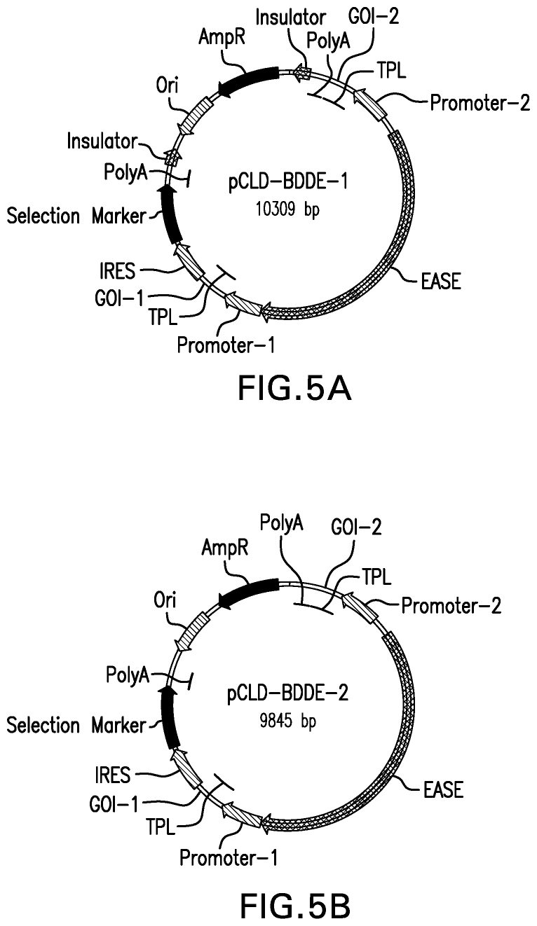

The DNA sequence information of plasmid pUC19 and various fragments, such as EASE, CMV Enhancer/Promoter, TPL, IRES, mammalian selection marker, and PolyA, were obtained from the public domain. The polynucleotides of pUC19 and these fragments were synthesized by Blue Heron Biotech, LLC, WA. A variety of pCLD-SE (FIG. 4) expression vectors with different mammalian selection markers were constructed, using NEBuilder HiFi DNA Assembly Cloning Kit (New England Biolabs, Beverly, Mass.). For the bi-directional vectors pCLD-BDDE-1 (FIG. 5A, with insulators) and pCLD-BDDE-2 (FIG. 5B, without insulators), reverse complement sequences for CMV E/P, TPL, and PolyA were synthesized (Blue Heron Biotech, LLC, WA) and constructed in the opposite orientation as indicated in FIGS. 5A and 5B. Exemplary antibody heavy chain and/or light chain sequences were synthesized (Blue Heron Biotech, LLC, WA) and cloned into pCLD-SE, pCLD-BDDE-1, or pCLD-BDDE-2.

The DNA sequences of exemplary elements or expression vectors are shown as follows:

IRES-1 (Genes Dev. 4(9):1560-72 (1990), SEQ ID NO: 1):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaacgtctaggccccccgaaccacggggacgtggttttcctttgaaaaacacgatgat
aatatggccacaacc
IRES-2 (SEQ ID NO: 2):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaacgtctaggccccccgaaccacggggacgtggttttcctttgaaaaacacgatgat
aa
IRES-3 (SEQ ID NO: 3):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaacgtctaggccccccgaaccacggggacgtggttttcctttgaaaaacacg
IRES-4 (SEQ ID NO: 25):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaaacgtctaggccccccgaaccacggggacgtggttttcctttgaaaaacacgatg
ataatatggccacaacc
IRES-5 (SEQ ID NO: 26):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaaacgtctaggccccccgaaccacggggacgtggttttcctttgaaaaacacgatg
ataa
IRES-6 (SEQ ID NO: 27):
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaaacgtctaggccccccgaaccacggggacgtggttttcctttgaaaaacacg
pCLD-BDDE-1A (with mammalian selection marker A, SEQ ID NO: 4):
ccaatgatcttaagttaatcgaatttgcagcccgggactagctagagggacagcccccccccaaagcccccagggatgtaattacgtccctc
ccccgctagggggcagcagcgagccgcccggggctccgctccggtccggcgctccccccgcatccccgagccggcagcgtgcgggg
acagcccgggcacggggaaggtggcacgggatcgctttcctctgaacgcttctcgctgctctttgagcctgcagacacctggggggatac
ggggaaaacttaagatccgaccggacgcgttctattaccacatttgtagaggttttacttgctttaaaaaacctcccacatctccccctgaacct
gaaacataaaatgaatgcaattgttgttgttaacttgtttattgcagcttataatggttacaaataaagcaatagcatcacaaatttcacaaataaa
gcatttttttcactgcattctagttgtggtttgtccaaactcatcaatgtatcttatgatgtgtgatcagttatctatgcggccgcggtggcggcgtc
gaccgagaggttttccgatccggtcgatgcggactcgctcaggtccctcggtggcggagtaccgttcggaggccgacgggtttccgatcc
aagagtactggaaagaccgcgaagagtttgtcctcaaccgcgagctgtggaaaaaaaagggacaggataagtatgacatcatcaaggaa
gcttgacaacaaaaagattgtcttttctgaccagatggacgcggccaccctcaaaggcatcaccgcgggccaggtgaatatcaaatcctcct
cgtttttggaaactgacaatcttagcgcagaagtcatgcccgcttttgagagggagtactcaccccaacagctggccctcgcagacagcgat
gcggaagaggatctgacggttcactaaacgagctctgcttatatagacctcccaccgtacacgcctaccgcccatttgcgtcaatggggcg
gagttgttacgacattttggaaagtcccgttgattttggtgccaaaacaaactcccattgacgtcaatggggtggagacttggaaatccccgtg
agtcaaaccgctatccacgcccattgatgtactgccaaaaccgcatcaccatggtaatagcgatgactaatacgtagatgtactgccaagtag
gaaagtcccataaggtcatgtactgggcataatgccaggcgggccatttaccgtcattgacgtcaataggggcgtacttggcatatgataca
cttgatgtactgccaagtgggcagtttaccgtaaatactccacccattgacgtcaatggaaagtccctattggcgttactatgggaacatacgt
cattattgacgtcaatgggcgggggtcgttgggcggtcagccaggcgggccatttaccgtaagttatgtaacactgacacacattccacagc
tgcctcgcgcgtttcggtgatgacggtgaaaacctctgacacatgcagctcccggagacggtcacagcttgtctgtaagcggatgccggga
gcagacaagcccgtcagggcgcgtcagcgggtgttggcgggtgtcggggcgcagccatgacccagtcacgtagcgatagcggagtgta
tactggcttaactatgcggcatcagagcagattgtactgagagcgctattctgaacttttcttttgttcccttcccttctaccacaccctaattgtaa
tccattttaatttcctggtcacagtcctgtctctccttccattgtaccttgcccttttctaaagagcgactgcaaagtatgtttgcgtaggtgaggat
ctaaaactttatgaggtacgaacatcacagaattactttgtaatttcagtttattgtaggcttggctttttggggagggtttacgtcttagacctctta
gtgcttctttgtttcatggtgttctaacttcgaagcatctctgtagctttaatggattccttttctgaaagctttgctctctttcttccccctcggctttct
cttaggcaagagggctaactgtaaagtaaggcttactgccttgtgtttccaaatgtgtccgaagaggaagtgtcttctgtgaatcctgttatgca
tgaataacaggaaatagaaagaaattcactttcattattataaaagtaatatgttcgtttaaaaaattctaatgaagagctggagatgcaaccca
ggggtagagcacacactcagcatgcaggaggccctgggtccaatcttggaatctcctctcagttaacctgatctctagctgattagtagtgag
tgcaagcccactttcctcttctgcctcattgctcagtgataacagctgttaaactttgtcttattctaaaactacctctgtgcaaatgctagcacaat
aatatatatcatatgcacatgattttttttttatcttgaaaagtaagtcagtatagctacaaagttcacttggcattgtcaacatttcacaggcgtaat
attcctcctctagtactgtcctcttcattctttgtgaccaagtttggagagagtgcacaaatgccagggaggtttgtgggaaggtttctcatgttct
ggtaaggcgagtaagaaaatagtctcatgcaggtgaaatgagtgctatgcagtatatattataccagagaacagcaaatgaccaaattcaca
ctgaactagttcagtaaaattggctttgtcaaagctttccttgcttaaaatgtaattccctgtcatcctagttctggtctggattcttttcctggagtct
tgacttccagattccctgtggacttttgtttgagtttcaagcttttgaaatatagaaacctatctaacttaacaaacttgggagagaaaagactcca
gaacaactgaaaacagaccaggctaaatgaatagactttattcctctcttcttacctgcagttttcagatatgcagagttggagcggatcttaga
ggttgattcattcatgcctgaagaaaacacattttatagaccctgtgcccaagttcgtggtggacatcaccctttatttactaattgcactacataa
caggcattttagaagactgctccagtcagagaccccgccttagaggaatctgtaaaccctgaactcctatcactcatgagcactagttatgttt
ggaatgccgtattaaaacaaaagttacatttctaaacttaaaattttctagcacagagacagtgggagtagctaactttgatagacatttttctact
aaaagtctttctaagtacataatcttctgtaagttggaaaacagcaaaatagaacgtctcctacgtagttaatctttttgcataatttgcacatgtag
gagttattagtatacgggtaagttttcactttttcccccaactggagtgtcttgtggctgggtttgaaaaagggaacgggaggccgctggagg
ggattggtaaatgagataaaacaccactcattcaactcagtgactcagcatttaaattttccataaaaggattaaaggaaaattaaacaaattctt
aaagccaagactctggagaaacttgttggtgtgattagttttcactgttatgactcatgaatttatgcataaattagtacatttataaaaacatagc
ctttttagagttttctgtttggctaaagtgccattgttagcatttggaattacctttttatgtcttatattttttccaaataaaaataaatgtttctgctgt
cttactactgaaactacgttgtgagcactttaaatttctcaaagcagtttcgcctgttatacttggcgcttagtcatcgtcgtacacaacaggacctga
ttaagaaggctgtgctgcctctaagccgggctagattgtagccactagcaaccaggctgcaataatttccctttgatgacatcatccactgtgg
aagaacccagttgcttcagccagtcgaactatacagttccaacctcatcaaatatggcatctcccttgcctgctatagcagggggaggaaaa
aatgccaccatctttttaatctagcaagcttctcttttcttcatctttttttttttcttttaaaaaaattctgatcatggatgcttcttccgatccctattt
gccttatgacgggggaggagacaatatccccttgagggaattacataaaagaggtaagagcatccccttgctctgaatcctctgttggttgttgtgc
atgcggctgggcggttctggggacaggctgtctgttgtcctcttgctgcaatgtgctgcttagttgccctgccttgttgctgtgggagaatgcg
accttcccagcagggctggccctccctgattgtttgctctgtgcagattagccctgcttcagatcacatagggctgcagactccatcttctgtgt
gaaaatgctttcggtttgattgcagaaataagctgcctttacagccagctaaagtcctggtggttggttggcacctgcaaagtagtatttttgtac
ctctggaaacttatattttctttacacagcaatatcaagtgccggtatgccattctgttttggctgctgccaattaccatgtagactttgcaccacag
agtaatagtaaaagctcctagctgcattttataacatttaaaaatagcaggaaagaagaattatttttgatttaacatgtttttgtcatttaacgtctta
actgattgacatactatattgtctgtctcgtgggtatcttgtacaacttgataggataaagcaatttagtttttttttttttttttaaatacatccagaatg
taagtcgtcagtagttttcgaacagataagtaatggtgttaatcttttggcaggctttgccttggtctccttaaagctaattaggtgttacttaattaaa
ctgctcttttgctcattttcttaaattatttttttaaaagatagttggcatttgctgttctagaaataaacttcaagaaacattctttagccagatgacttc
atgtatgagccatgttagtttgaattatttgcttggtgttataaactttatggtttaataccaacttttattatgtttacaaggtaaataaggaaaatttc
aagtacattttgtatcctgagaacaaatttaagttccatagaatttaggaattacaatgtattcaacagatacttacttgtcatactgtgcctgcaaa
acaataattagactctgaacaggtgcaacaattttctgtagaattagacaagtcttcttttggcaggtgttactaagtaggccatttcccaaggaa
cagggaatttgccaggcttttgtggtggagagaatagaatgaataaatgctgtggggagtaaagagcttgtcagaagatgattagttctgtgg
caccaaaaccaagagatcagttttcctgtgagaagtaaaggaagcattgtagaaaaatagatgtgttgaagtctaccggtggagttccgcgtt
acataacttacggtaaatggcccgcctggctgaccgcccaacgacccccgcccattgacgtcaataatgacgtatgttcccatagtaacgcc
aatagggactttccattgacgtcaatgggtggagtatttacggtaaactgcccacttggcagtacatcaagtgtatcatatgccaagtacgccc
cctattgacgtcaatgacggtaaatggcccgcctggcattatgcccagtacatgaccttatgggactttcctacttggcagtacatctacgtatt
agtcatcgctattaccattgtgatgcggttttggcagtacatcaatgggcgtggatagcggtttgactcacggggatttccaagtctccacccc
attgacgtcaatgggagtttgttttggcaccaaaatcaacgggactttccaaaatgtcgtaacaactccgccccattgacgcaaatgggcggt
aggcgtgtacggtgggaggtctatataagcagagctcgtttagtgaaccgtcagatctacctcttccgcatcgctgtctgcgagggccagct
gttggggtgagtactccctctcaaaagcgggcatgacttctgcgctaagattgtcagtttccaaaaacgaggaggatttgatattcacctggcc
cgcggtgatgcctttgagggtggccgcgtccatctggtcagaaaagacaatctttttgttgtcaagcttccttgatgatgtcatacttatcctgtc
ccttttttttccacagctcgcggttgaggacaaactcttcgcggtctttccagtactcttggatcggaaacccgtcggcctccgaacggtactcc
gccaccgagggacctgagcgagtccgcatcgaccggatcggaaaacctcggatccgccgccaccgaattcatagataactgatccagtg
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaacgtctaggccccccgaaccacggggacgtggttttcctttgaaaaacacgatgat
aagcttgccacaacccacaaggagacgaccttccatgattgaacaagatggattgcacgcaggttctccggccgcttgggtggagaggct
attcggctatgactgggcacaacagacaatcggctgctctgatgccgccgtgttccggctgtcagcgcaggggcgcccggttctttttgtca
agaccgacctgtccggtgccctgaatgaactgcaggacgaggcagcgcggctatcgtggctggccacgacgggcgttccttgcgcagct
gtgctcgacgttgtcactgaagcgggaagggactggctgctattgggcgaagtgccggggcaggatctcctgtcatctcaccttgctcctgc
cgagaaagtatccatcatggctgatgcaatgcggcggctgcatacgcttgatccggctacctgcccattcgaccaccaagcgaaacatcgc
atcgagcgagcacgtactcggatggaagccggtcttgtcgatcaggatgatctggacgaggagcatcaggggctcgcgccagccgaact
gttcgccaggctcaaggcgcgcatgcccgacggcgaggatctcgtcgtgacccatggcgatgcctgcttgccgaatatcatggtggaaaa
tggccgcttttctggattcatcgactgtggccggctgggtgtggcggaccgctatcaggacatagcgttggctacccgtgatattgctgaaga
acttggcggcgaatgggctgaccgcttcctcgtgctttacggtatcgccgctcccgattcgcagcgcatcgccttctatcgccttcttgacgag
ttcttctgatctagatccccctcgctttcttgctgtccaatttctattaaaggttcctttgttccctaagtccaactactaaactgggggatattatgaa
gggccttgagcatctggattctgcctaataaaaaacatttattttcattgcaatgatgtatttaaattatttctgaatattttactaaaaagggaatgt
gggaggtcagtgcatttaaaacataaagaaatgaagagggggatcttcgcgatactgcatcgatgagggacagcccccccccaaagccc
ccagggatgtaattacgtccctcccccgctagggggcagcagcgagccgcccggggctccgctccggtccggcgctccccccgcatcc
ccgagccggcagcgtgcggggacagcccgggcacggggaaggtggcacgggatcgctttcctctgaacgcttctcgctgctctttgagc
ctgcagacacctggggggatacggggaaaatagacaccgcggtggagctccagcttttgttccctttagtgagggttaattagttcttaatac
gactcactatagggcgaattggctaccgggccgcccatcgagggtatcataagcttttaaatcgatagatgcgatatcggaaagaacatgtg
agcaaaaggccagcaaaaggccaggaaccgtaaaaaggccgcgttgctggcgtttttccataggctccgcccccctgacgagcatcaca
aaaatcgacgctcaagtcagaggtggcgaaacccgacaggactataaagataccaggcgtttccccctggaagctccctcgtgcgctctcc
tgttccgaccctgccgcttaccggatacctgtccgcctttctcccttcgggaagcgtggcgctttctcatagctcacgctgtaggtatctcagtt
cggtgtaggtcgttcgctccaagctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggtatgtaggcggtgctacagagt
tcttgaagtggtggcctaactacggctacactagaagaacagtatttggtatctgcgctctgctgaagccagttaccttcggaaaaagagttgg
tagctcttgatccggcaaacaaaccaccgctggtagcggtggtttttttgtttgcaagcagcagattacgcgcagaaaaaaaggatctcaaga
agatcctttgatcttttctacggggtctgacgctcagtggaacgaaaactcacgttaagggattttggtcatgagattatcaaaaaggatcttca
cctagatccttttaaattaaaaatgaagttttaaatcaatctaaagtatatatgagtaaacttggtctgacagttaccaatgcttaatcagtgaggc
acctatctcagcgatctgtctatttcgttcatccatagttgcctgactccccgtcgtgtagataactacgatacgggagggcttaccatctggcc
ccagtgctgcaatgataccgcgagacccacgctcaccggctccagatttatcagcaataaaccagccagccggaagggccgagcgcaga
agtggtcctgcaactttatccgcctccatccagtctattaattgttgccgggaagctagagtaagtagttcgccagttaatagtttgcgcaacgtt
gttgccattgctacaggcatcgtggtgtcacgctcgtcgtttggtatggcttcattcagctccggttcccaacgatcaaggcgagttacatgatc
ccccatgttgtgcaaaaaagcggttagctccttcggtcctccgatcgttgtcagaagtaagttggccgcagtgttatcactcatggttatggca
gcactgcataattctcttactgtcatgccatccgtaagatgcttttctgtgactggtgagtactcaaccaagtcattctgagaatagtgtatgcgg
cgaccgagttgctcttgcccggcgtcaatacgggataataccgcgccacatagcagaactttaaaagtgctcatcattggaaaacgttcttcg
gggcgaaaactctcaaggatcttaccgctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacggaaatgttgaatactcatactctt
cctttttcaatattattgaagcatttatcagggttattgtctcatgagcggatacatatttgaatgtatttagaaaaataaacaaataggggttccgc
gcacatttccccgaaaagtgccacctgacgtc
pCLD-BDDE-1B (with mammalian selection marker B, SEQ ID NO: 5):
ccaatgatcttaagttaatcgaatttgcagcccgggactagctagagggacagcccccccccaaagcccccagggatgtaattacgtccctc
ccccgctagggggcagcagcgagccgcccggggctccgctccggtccggcgctccccccgcatccccgagccggcagcgtgcgggg
acagcccgggcacggggaaggtggcacgggatcgctttcctctgaacgcttctcgctgctctttgagcctgcagacacctggggggatac
ggggaaaacttaagatccgaccggacgcgttctattaccacatttgtagaggttttacttgctttaaaaaacctcccacatctccccctgaacct
gaaacataaaatgaatgcaattgttgttgttaacttgtttattgcagcttataatggttacaaataaagcaatagcatcacaaatttcacaaataaa
gcatttttttcactgcattctagttgtggtttgtccaaactcatcaatgtatcttatgatgtgtgatcagttatctatgcggccgcggtggcggcgtc
gaccgagaggttttccgatccggtcgatgcggactcgctcaggtccctcggtggcggagtaccgttcggaggccgacgggtttccgatcc
aagagtactggaaagaccgcgaagagtttgtcctcaaccgcgagctgtggaaaaaaaagggacaggataagtatgacatcatcaaggaa
gcttgacaacaaaaagattgtcttttctgaccagatggacgcggccaccctcaaaggcatcaccgcgggccaggtgaatatcaaatcctcct
cgtttttggaaactgacaatcttagcgcagaagtcatgcccgcttttgagagggagtactcaccccaacagctggccctcgcagacagcgat
gcggaagaggatctgacggttcactaaacgagctctgcttatatagacctcccaccgtacacgcctaccgcccatttgcgtcaatggggcg
gagttgttacgacattttggaaagtcccgttgattttggtgccaaaacaaactcccattgacgtcaatggggtggagacttggaaatccccgtg
agtcaaaccgctatccacgcccattgatgtactgccaaaaccgcatcaccatggtaatagcgatgactaatacgtagatgtactgccaagtag
gaaagtcccataaggtcatgtactgggcataatgccaggcgggccatttaccgtcattgacgtcaataggggcgtacttggcatatgataca
cttgatgtactgccaagtgggcagtttaccgtaaatactccacccattgacgtcaatggaaagtccctattggcgttactatgggaacatacgt
cattattgacgtcaatgggcgggggtcgttgggcggtcagccaggcgggccatttaccgtaagttatgtaacactgacacacattccacagc
tgcctcgcgcgtttcggtgatgacggtgaaaacctctgacacatgcagctcccggagacggtcacagcttgtctgtaagcggatgccggga
gcagacaagcccgtcagggcgcgtcagcgggtgttggcgggtgtcggggcgcagccatgacccagtcacgtagcgatagcggagtgta
tactggcttaactatgcggcatcagagcagattgtactgagagcgctattctgaacttttcttttgttcccttcccttctaccacaccctaattgtaa
tccattttaatttcctggtcacagtcctgtctctccttccattgtaccttgcccttttctaaagagcgactgcaaagtatgtttgcgtaggtgaggat
ctaaaactttatgaggtacgaacatcacagaattactttgtaatttcagtttattgtaggcttggctttttggggagggtttacgtcttagacctctta
gtgcttctttgtttcatggtgttctaacttcgaagcatctctgtagctttaatggattccttttctgaaagctttgctctctttcttccccctcggctttct
cttaggcaagagggctaactgtaaagtaaggcttactgccttgtgtttccaaatgtgtccgaagaggaagtgtcttctgtgaatcctgttatgca
tgaataacaggaaatagaaagaaattcactttcattattataaaagtaatatgttcgtttaaaaaattctaatgaagagctggagatgcaaccca
ggggtagagcacacactcagcatgcaggaggccctgggtccaatcttggaatctcctctcagttaacctgatctctagctgattagtagtgag
tgcaagcccactttcctcttctgcctcattgctcagtgataacagctgttaaactttgtcttattctaaaactacctctgtgcaaatgctagcacaat
aatatatatcatatgcacatgattttttttttatcttgaaaagtaagtcagtatagctacaaagttcacttggcattgtcaacatttcacaggcgtaat
attcctcctctagtactgtcctcttcattctttgtgaccaagtttggagagagtgcacaaatgccagggaggtttgtgggaaggtttctcatgttct
ggtaaggcgagtaagaaaatagtctcatgcaggtgaaatgagtgctatgcagtatatattataccagagaacagcaaatgaccaaattcaca
ctgaactagttcagtaaaattggctttgtcaaagctttccttgcttaaaatgtaattccctgtcatcctagttctggtctggattcttttcctggagtct
tgacttccagattccctgtggacttttgtttgagtttcaagcttttgaaatatagaaacctatctaacttaacaaacttgggagagaaaagactcca
gaacaactgaaaacagaccaggctaaatgaatagactttattcctctcttcttacctgcagttttcagatatgcagagttggagcggatcttaga
ggttgattcattcatgcctgaagaaaacacattttatagaccctgtgcccaagttcgtggtggacatcaccctttatttactaattgcactacataa
caggcattttagaagactgctccagtcagagaccccgccttagaggaatctgtaaaccctgaactcctatcactcatgagcactagttatgttt
ggaatgccgtattaaaacaaaagttacatttctaaacttaaaattttctagcacagagacagtgggagtagctaactttgatagacatttttctact
aaaagtctttctaagtacataatcttctgtaagttggaaaacagcaaaatagaacgtctcctacgtagttaatctttttgcataatttgcacatgtag
gagttattagtatacgggtaagttttcactttttcccccaactggagtgtcttgtggctgggtttgaaaaagggaacgggaggccgctggagg
ggattggtaaatgagataaaacaccactcattcaactcagtgactcagcatttaaattttccataaaaggattaaaggaaaattaaacaaattctt
aaagccaagactctggagaaacttgttggtgtgctttagttttcactgttatgactcatgaatttatgcataaattagtacatttataaaaacatagc
ctttttagagttttctgtttggctaaagtgccattgttagcatttggaattacctttttatgtcttatattttttccaaataaaaataaatgtttctgctgt
cttactactgaaactacgttgtgagcactttaaatttctcaaagcagtttcgcctgttatacttggcgcttagtcatcgtcgtacacaacaggacctga
ttaagaaggctgtgctgcctctaagccgggctagattgtagccactagcaaccaggctgcaataatttccctttgatgacatcatccactgtgg
aagaacccagttgcttcagccagtcgaactatacagttccaacctcatcaaatatggcatctcccttgcctgctatagcagggggaggaaaa
aatgccaccatctttttaatctagcaagcttctcttttcttcatctttttttttttcttttaaaaaaattctgatcatggatgcttcttccgatccctattt
gccttatgacgggggaggagacaatatccccttgagggaattacataaaagaggtaagagcatccccttgctctgaatcctctgttggttgttgtgc
atgcggctgggcggttctggggacaggctgtctgttgtcctcttgctgcaatgtgctgcttagttgccctgccttgttgctgtgggagaatgcg
accttcccagcagggctggccctccctgattgtttgctctgtgcagattagccctgcttcagatcacatagggctgcagactccatcttctgtgt
gaaaatgctttcggtttgattgcagaaataagctgcctttacagccagctaaagtcctggtggttggttggcacctgcaaagtagtatttttgtac
ctctggaaacttatattttctttacacagcaatatcaagtgccggtatgccattctgttttggctgctgccaattaccatgtagactttgcaccacag
agtaatagtaaaagctcctagctgcattttataacatttaaaaatagcaggaaagaagaattatttttgatttaacatgtttttgtcatttaacgtctta
actgattgacatactatattgtctgtctcgtgggtatcttgtacaacttgataggataaagcaatttagtttttttttttttttttaaatacatccagaatg
taagtcgtcagtagttttcgaacagataagtaatggtgttaatcttttggcaggctttgccttggtctccttaaagctaattaggtgttacttaattaaa
ctgctcttttgctcattttcttaaattatttttttaaaagatagttggcatttgctgttctagaaataaacttcaagaaacattctttagccagatgacttc
atgtatgagccatgttagtttgaattatttgcttggtgttataaactttatggtttaataccaacttttattatgtttacaaggtaaataaggaaaatttc
aagtacattttgtatcctgagaacaaatttaagttccatagaatttaggaattacaatgtattcaacagatacttacttgtcatactgtgcctgcaaa
acaataattagactctgaacaggtgcaacaattttctgtagaattagacaagtcttcttttggcaggtgttactaagtaggccatttcccaaggaa
cagggaatttgccaggcttttgtggtggagagaatagaatgaataaatgctgtggggagtaaagagcttgtcagaagatgattagttctgtgg
caccaaaaccaagagatcagttttcctgtgagaagtaaaggaagcattgtagaaaaatagatgtgttgaagtctaccggtggagttccgcgtt
acataacttacggtaaatggcccgcctggctgaccgcccaacgacccccgcccattgacgtcaataatgacgtatgttcccatagtaacgcc
aatagggactttccattgacgtcaatgggtggagtatttacggtaaactgcccacttggcagtacatcaagtgtatcatatgccaagtacgccc
cctattgacgtcaatgacggtaaatggcccgcctggcattatgcccagtacatgaccttatgggactttcctacttggcagtacatctacgtatt
agtcatcgctattaccattgtgatgcggttttggcagtacatcaatgggcgtggatagcggtttgactcacggggatttccaagtctccacccc
attgacgtcaatgggagtttgttttggcaccaaaatcaacgggactttccaaaatgtcgtaacaactccgccccattgacgcaaatgggcggt
aggcgtgtacggtgggaggtctatataagcagagctcgtttagtgaaccgtcagatctacctcttccgcatcgctgtctgcgagggccagct
gttggggtgagtactccctctcaaaagcgggcatgacttctgcgctaagattgtcagtttccaaaaacgaggaggatttgatattcacctggcc
cgcggtgatgcctttgagggtggccgcgtccatctggtcagaaaagacaatctttttgttgtcaagcttccttgatgatgtcatacttatcctgtc
ccttttttttccacagctcgcggttgaggacaaactcttcgcggtctttccagtactcttggatcggaaacccgtcggcctccgaacggtactcc
gccaccgagggacctgagcgagtccgcatcgaccggatcggaaaacctcggatccgccgccaccgaattcatagataactgatccagtg
cccctaacgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctctcgccaaaggaatgcaaggtctgttgaatgtcgtga
aggaagcagttcctctggaagcttcttgaagacaaacaacgtctgtagcgaccctttgcaggcagcggaaccccccacctggcgacaggt
gcctctgcggccaaaagccacgtgtataagatacacctgcaaaggcggcacaaccccagtgccacgttgtgagttggatagttgtggaaa
gagtcaaatggctctcctcaagcgtattcaacaaggggctgaaggatgcccagaaggtaccccattgtatgggatctgatctggggcctcg
gtgcacatgctttacatgtgtttagtcgaggttaaaaaacgtctaggccccccgaaccacggggacgtggttttcctttgaaaaacacgatgat
aagcttgccacaacccacaaggagacgaccttccatgaccgagtacaagcccacggtgcgcctcgccacccgcgacgacgtcccccgg
gccgtacgcaccctcgccgccgcgttcgccgactaccccgccacgcgccacaccgtcgacccggaccgccacatcgagcgggtcacc
gagctgcaagaactcttcctcacgcgcgtcgggctcgacatcggcaaggtgtgggtcgcggacgacggcgccgcggtggcggtctgga
ccacgccggagagcgtcgaagcgggggcggtgttcgccgagatcggcccgcgcatggccgagttgagcggttcccggctggccgcgc
agcaacagatggaaggcctcctggcgccgcaccggcccaaggagcccgcgtggttcctggccaccgtcggcgtctcgcccgaccacca
gggcaagggtctgggcagcgccgtcgtgctccccggagtggaggcggccgagcgcgccggggtgcccgccttcctggagacctccgc
gccccgcaacctccccttctacgagcggctcggcttcaccgtcaccgccgacgtcgaggtgcccgaaggaccgcgcacctggtgcatga
cccgcaagcccggtgcctctagatccccctcgctttcttgctgtccaatttctattaaaggttcctttgttccctaagtccaactactaaactggg
ggatattatgaagggccttgagcatctggattctgcctaataaaaaacatttattttcattgcaatgatgtatttaaattatttctgaatattttactaa
aaagggaatgtgggaggtcagtgcatttaaaacataaagaaatgaagagggggatcttcgcgatactgcatcgatgagggacagcccccc
cccaaagcccccagggatgtaattacgtccctcccccgctagggggcagcagcgagccgcccggggctccgctccggtccggcgctcc
ccccgcatccccgagccggcagcgtgcggggacagcccgggcacggggaaggtggcacgggatcgctttcctctgaacgcttctcgct
gctctttgagcctgcagacacctggggggatacggggaaaatagacaccgcggtggagctccagcttttgttccctttagtgagggttaatta
gttcttaatacgactcactatagggcgaattggctaccgggccgcccatcgagggtatcataagcttttaaatcgatagatgcgatatcggaa
agaacatgtgagcaaaaggccagcaaaaggccaggaaccgtaaaaaggccgcgttgctggcgtttttccataggctccgcccccctgac
gagcatcacaaaaatcgacgctcaagtcagaggtggcgaaacccgacaggactataaagataccaggcgtttccccctggaagctccctc
gtgcgctctcctgttccgaccctgccgcttaccggatacctgtccgcctttctcccttcgggaagcgtggcgctttctcatagctcacgctgtag
gtatctcagttcggtgtaggtcgttcgctccaagctgggctgtgtgcacgaaccccccgttcagcccgaccgctgcgccttatccggtaacta
tcgtcttgagtccaacccggtaagacacgacttatcgccactggcagcagccactggtaacaggattagcagagcgaggtatgtaggcggt
gctacagagttcttgaagtggtggcctaactacggctacactagaagaacagtatttggtatctgcgctctgctgaagccagttaccttcggaa
aaagagttggtagctcttgatccggcaaacaaaccaccgctggtagcggtggtttttttgtttgcaagcagcagattacgcgcagaaaaaaa
ggatctcaagaagatcctttgatcttttctacggggtctgacgctcagtggaacgaaaactcacgttaagggattttggtcatgagattatcaaa
aaggatcttcacctagatccttttaaattaaaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtgtagataactacgatacgggagggctta
ccatctggccccagtgctgcaatgataccgcgagacccacgctcaccggctccagatttatcagcaataaaccagccagccggaagggcc
gagcgcagaagtggtcctgcaactttatccgcctccatccagtctattaattgttgccgggaagctagagtaagtagttcgccagttaatagttt
gcgcaacgttgttgccattgctacaggcatcgtggtgtcacgctcgtcgtttggtatggcttcattcagctccggttcccaacgatcaaggcga
gttacatgatcccccatgttgtgcaaaaaagcggttagctccttcggtcctccgatcgttgtcagaagtaagttggccgcagtgttatcactcat
ggttatggcagcactgcataattctcttactgtcatgccatccgtaagatgcttttctgtgactggtgagtactcaaccaagtcattctgagaata
gtgtatgcggcgaccgagttgctcttgcccggcgtcaatacgggataataccgcgccacatagcagaactttaaaagtgctcatcattggaa
aacgttcttcggggcgaaaactctcaaggatcttaccgctgttgagatccagttcgatgtaacccactcgtgcacccaactgatcttcagcatc
ttttactttcaccagcgtttctgggtgagcaaaaacaggaaggcaaaatgccgcaaaaaagggaataagggcgacacggaaatgttgaata
ctcatactcttcctttttcaatattattgaagcatttatcagggttattgtctcatgagcggatacatatttgaatgtatttagaaaaataaacaaatag
gggttccgcgcacatttccccgaaaagtgccacctgacgtc
5′ ITR (Virology 172 (1), 156-169 (1989), GenBank: J04364.2, SEQ ID NO: 6):
ccctagaaagataatcatattgtgacgtacgttaaagataatcatgcgtaaaattgacgcatg
3′ ITR (Virology 172 (1), 156-169 (1989), GenBank: J04364.2, SEQ ID NO: 7):
catgcgtcaattttacgcagactatctttctaggg
HS4 Insulator (Cell 74(3):505-14 (1993), Proc Natl Acad Sci USA. 94(2):575-80 (1997),
GenBank: U78775.2, SEQ ID NO: 8):
gagctcacggggacagcccccccccaaagcccccagggatgtaattacgtccctcccccgctagggggcagcagcgagccgcccggg
gctccgctccggtccggcgctccccccgcatccccgagccggcagcgtgcggggacagcccgggcacggggaaggtggcacgggat
cgctttcctctgaacgcttctcgctgctctttgagcctgcagacacctggggggatacggggaaaaagctttaggctgaaagagagatttaga
atgacagaatcatagaacggcctgggttgcaaaggagcacagtgctcatccagatccaaccccctgctatgtgcagggtcatcaaccagca
gcccaggctgcccagagccacatccagcctggccttgaatgcctgcagggatggggcatccacagcctccttgggcaacctgttcagtgc
gtcaccaccctctgggggaaaaactgcctcctcatatccaacccaaacctcccctgtctcagtgtaaagccattcccccttgtcctatcaagg
gggagtttgctgtgacattgttggtctggggtgacacatgtttgccaattcagtgcatcacggagaggcagatcttggggataaggaagtgca
ggacagcatggacgtgggacatgcaggtgttgagggctctgggacactctccaagtcacagcgttcagaacagccttaaggataagaaga
taggatagaaggacaaagagcaagttaaaacccagcatggagaggagcacaaaaaggccacagacactgctggtccctgtgtctgagcc
tgcatgtttgatggtgtctggatgcaagcagaaggggtggaagagcttgcctggagagatacagctgggtcagtaggactgggacaggca
gctggagaattgccatgtagatgttcatacaatcgtcaaatcatgaaggctggaaaagccctccaagatccccaagaccaaccccaaccca
cccaccgtgcccactggccatgtccctcagtgccacatccccacagttcttcatcacctccagggacggtgacccccccacctccgtgggc
agctgtgccactgcagcaccgctctttggagaaggtaaatcttgctaaatccagcccgaccctcccctggcacaacgtaaggccattatctct
catccaactccaggacggagtcagtgagaatatt
EASE (Cytotechnology 28(1-3):9-17 (1998), GenBank: AF193761.1, SEQ ID NO: 9):
gaattctgaacttttcttttgttcccttcccttctaccacaccctaattgtaatccattttaatttcctggtcacagtcctgtctctccttccattgtacct
tgcccttttctaaagagcgactgcaaagtatgtttgcgtaggtgaggatctaaaactttatgaggtacgaacatcacagaattactttgtaatttca
gtttattgtaggcttggctttttggggagggtttacgtcttagacctcttagtgcttctttgtttcatggtgttctaacttcgaagcatctctgtagcttt
aatggattccttttctgaaagctttgctctctttcttccccctcggctttctcttaggcaagagggctaactgtaaagtaaggcttactgccttgtgtt
tccaaatgtgtccgaagaggaagtgtcttctgtgaatcctgttatgcatgaataacaggaaatagaaagaaattcactttcattattataaaagta
atatgttcgtttaaaaaattctaatgaagagctggagatgcaacccaggggtagagcacacactcagcatgcaggaggccctgggtccaat
cttggaatctcctctcagttaacctgatctctagctgattagtagtgagtgcaagcccactttcctcttctgcctcattgctcagtgataacagctg
ttaaactttgtcttattctaaaactacctctgtgcaaatgctagcacaataatatatatcatatgcacatgattttttttttatcttgaaaagtaagtcag
tatagctacaaagttcacttggcattgtcaacatttcacaggcgtaatattcctcctctagtactgtcctcttcattctttgtgaccaagtttggaga
gagtgcacaaatgccagggaggtttgtgggaaggtttctcatgttctggtaaggcgagtaagaaaatagtctcatgcaggtgaaatgagtgc
tatgcagtatatattataccagagaacagcaaatgaccaaattcacactgaactagttcagtaaaattggctttgtcaaagctttccttgcttaaa
atgtaattccctgtcatcctagttctggtctggattcttttcctggagtcttgacttccagattccctgtggacttttgtttgagtttcaagcttttgaaa
tatagaaacctatctaacttaacaaacttgggagagaaaagactccagaacaactgaaaacagaccaggctaaatgaatagactttattcctc
tcttcttacctgcagttttcagatatgcagagttggagcggatcttagaggttgattcattcatgcctgaagaaaacacattttatagaccctgtgc
ccaagttcgtggtggacatcaccctttatttactaattgcactacataacaggcattttagaagactgctccagtcagagaccccgccttagag
gaatctgtaaaccctgaactcctatcactcatgagcactagttatgtttggaatgccgtattaaaacaaaagttacatttctaaacttaaaattttct
agcacagagacagtgggagtagctaactttgatagacatttttctactaaaagtctttctaagtacataatcttctgtaagttggaaaacagcaaa
atagaacgtctcctacgtagttaatctttttgcataatttgcacatgtaggagttattagtatacgggtaagttttcactttttcccccaactggagtg
tcttgtggctgggtttgaaaaagggaacgggaggccgctggaggggattggtaaatgagataaaacaccactcattcaactcagtgactca
gcatttaaattttccataaaaggattaaaggaaaattaaacaaattcttaaagccaagactctggagaaacttgttggtgtgctttagttttcactgt
tatgactcatgaatttatgcataaattagtacatttataaaaacatagcctttttagagttttctgtttggctaaagtgccattgttagcatttggaatta
cctttttatgtcttatattttttccaaataaaaataaatgtttctgctgtcttactactgaaactacgttgtgagcactttaaatttctcaaagcagtttcg
cctgttatacttggcgcttagtcatcgtcgtacacaacaggacctgattaagaaggctgtgctgcctctaagccgggctagattgtagccacta
gcaaccaggctgcaataatttccctttgatgacatcatccactgtggaagaacccagttgcttcagccagtcgaactatacagttccaacctca
tcaaatatggcatctcccttgcctgctatagcagggggaggaaaaaatgccaccatctttttaatctagcaagcttctcttttcttcatcatttttttt
tcttttaaaaaaattctgatcatggatgcttcttccgatccctatttgccttatgacgggggaggagacaatatccccttgagggaattacataaa
agaggtaagagcatccccttgctctgaatcctctgttggttgttgtgcatgcggctgggcggttctggggacaggctgtctgttgtcctcttgct
gcaatgtgctgcttagttgccctgccttgttgctgtgggagaatgcgaccttcccagcagggctggccctccctgattgtttgctctgtgcagat
tagccctgcttcagatcacatagggctgcagactccatcttctgtgtgaaaatgctttcggtttgattgcagaaataagctgcctttacagccag
ctaaagtcctggtggttggttggcacctgcaaagtagtatttttgtacctctggaaacttatattttctttacacagcaatatcaagtgccggtatg
ccattctgttttggctgctgccaattaccatgtagactttgcaccacagagtaatagtaaaagctcctagctgcattttataacatttaaaaatagc
aggaaagaagaattatttttgatttaacatgtttttgtcatttaacgtcttaactgattgacatactatattgtctgtctcgtgggtatcttgtacaactt
gataggataaagcaatttagattttttttttttttaaatacatccagaatgtaagtcgtcagtagttttcgaacagataagtaatggtgttaatcttttg
gcaggctttgccttggtctccttaaagctaattaggtgttacttaattaaactgctcttttgctcattttcttaaattatttttttaaaagatagttggcat
ttgctgttctagaaataaacttcaagaaacattctttagccagatgacttcatgtatgagccatgttagtttgaattatttgcttggtgttataaacttta
tggtttaataccaacttttattatgtttacaaggtaaataaggaaaatttcaagtacattttgtatcctgagaacaaatttaagttccatagaatttag
gaattacaatgtattcaacagatacttacttgtcatactgtgcctgcaaaacaataattagactctgaacaggtgcaacaattttctgtagaattct
gtgcttagtaaaaggttgctttttatattttgagagaaatctatttaaagatcatggcccatattttgtgcatatttttttctgtataccatttccatatat
gtgtgtgtgtgtacatatatgtatatatataaaatgttagaacatttgaggaaatagctaaaagtacaaaagtaatgttttctaattttttactccccga
ggttatttcttttttccttgttttcctttctctttgttcctatcatcagtttctagtaatactcttattgaacagtgattattcaaatgtcacattatttat
taatcagcatttaaatggtaaaaccagacagaccatactttctctgagtgatgacaacatccatttttagtaatgataaactagaagggtcaggcttgat
agtctttgtcaggactaatttatagactgtaaaggccaaaagaaataagaaatgtcaaaactcttgtgaaactagacatacagatattaccaag
agagaaactagaaaaaaaaattctgtgacatggccttaatttgccaggcaccatcgtgaaggcctaaacccctcttagaagctcactcagatg
ccatcctgcttctctgatgagacttcctgtcaatacaaacatggtttaggaagaatgagtgtttgcagtataaaccagttatttactagccttacttt
aagaatatactgtagtgtccttgagagagaaggtgtttgttttctgtaatttatgacccttttgaaaccatagatcagcacaaaggaactggggat
atggaaatgggaacataacttaaatccagaaaagtgaatcagattccctgtgaggacaaaatgcaatatttagaaataggatctttaggctgg
gagggagaaaagaggaaaaaaatgaaagtataacatttttcataagtataagatttcataaaaaaatgaaatctataacatagagggtgttgat
aaagtaagcatggatatgtttagtaaagccgacagagctaagaattagctttgtgagtaattggacttaatcaaacttttcaaggtgggatacaa
atgaataattgtagaataaatggataaaagaatatgaataaaatgaatagtgagtaaaaattaaaaatgaagctttttacttaagtgcatattgta
gtggctagaacaaatagattcaaaatagaaatcatttatatattcttgattagaagataaaatgttattttagaaatagccatctttggaagtaaattt
gctatgttgaacaaccaggttttcataatttgtctcttattttttttcaggaagaaaaaaaaacttgacttatttgtactgctaagttttattcaatgtgct
tgcttgcttaaatttttaatgaagttttagtcatttggtggtcaaattccttttatctactaatcgcttttcgtggctttggcttttaaaattgtatttact
gcatttatttgtgtgtattaggagtcaggtggccatatgtgccatggcatgtgtatggaagcacttgtggacatgaatcttctctttccacatgtgtgg
gccactggaatcaaactagtgtcaggcttggcagcaatttttaatgcactgagccttctcaccaccccctggacttttgtggggcagaagggg
acaagtttaatattttatttactccatgtagaaagcctttaaaaaatgtagaaagcctttaaactacctattgttttatttgaattatgaagctcttgtgt
ttatataaattacagttaggtactgtggagactaatggtagctacaatagtaatattaatagctaaaacttagtagaatctgattgagttaatttggc
cctttccatcataaggtactcttcccaagcatcacatgacctgtgcttaagtctggtgggggcttatggctttgatattgaaaacaaatcgtcaag
gatgttaatttcttgttactgctattacactgaattttctatggctctttaggagaggaagagacaagtcttcttttggcaggtgttactaagtaggc
catttcccaaggaacagggaatttgccaggcttttgtggtggagagaatagaatgaataaatgctgtggggagtaaagagcttgtcagaaga
tgattagttctgtggcaccaaaaccaagagatcagttttcctgtgagaagtaaaggaagcattgtagaaaaatagatgtgttgaagtct
Beta-globin polyA (Proc Natl Acad Sci USA 87(10):3924-8 (1990), GenBank: AH001475,
SEQ ID NO: 10):
tgccctggcccacaagtatcactaagctcgctttcttgctgtccaatttctattaaaggttcctttgttccctaagtccaactactaaactggggga
tattatgaagggccttgagcatctggattctgcctaataaaaaacatttattttcattgcaatgatgtatttaaattatttctgaatattttactaaaaag
ggaatgtgggaggtcagtgcatttaaaacataaagaaatgaagagctagttcaaaccttgggaaaatacactatatcttaaa
hCMV immediate-early enhancer/promoter (derived from GenBank X17403.1, SEQ ID
NO: 11):
ggagttccgcgttacataacttacggtaaatggcccgcctggctgaccgcccaacgacccccgcccattgacgtcaataatgacgtatgttc
ccatagtaacgccaatagggactttccattgacgtcaatgggtggagtatttacggtaaactgcccacttggcagtacatcaagtgtatcatat
gccaagtacgccccctattgacgtcaatgacggtaaatggcccgcctggcattatgcccagtacatgaccttatgggactttcctacttggca
gtacatctacgtattagtcatcgctattaccatggtgatgcggttttggcagtacatcaatgggcgtggatagcggtttgactcacggggatttc
caagtctccaccccattgacgtcaatgggagtttgttttggcaccaaaatcaacgggactttccaaaatgtcgtaacaactccgccccattgac
gcaaatgggcggtaggcgtgtacggtgggaggtctatataagcagagctcgtttagtgaaccgtcagatc
TPL (SEQ ID NO: 12):
ctcttccgcatcgctgtctgcgagggccagctgttggggtgagtactccctctcaaaagcgggcatgacttctgcgctaagattgtcagtttcc
aaaaacgaggaggatttgatattcacctggcccgcggtgatgcctttgagggtggccgcgtccatctggtcagaaaagacaatctttttgttgt
caagcttccttgatgatgtcatacttatcctgtcccttttttttccacagctcgcggttgaggacaaactcttcgcggtctttccagtactcttggatc
ggaaacccgtcggcctccgaacggtactccgccaccgagggacctgagcgagtccgcatcgaccggatcggaaaacctc
pUC replication origin Ori (SEQ ID NO: 13):
tttccataggctccgcccccctgacgagcatcacaaaaatcgacgctcaagtcagaggtggcgaaacccgacaggactataaagatacca
ggcgtttccccctggaagctccctcgtgcgctctcctgttccgaccctgccgcttaccggatacctgtccgcctttctcccttcgggaagcgtg
gcgctttctcatagctcacgctgtaggtatctcagttcggtgtaggtcgttcgctccaagctgggctgtgtgcacgaaccccccgttcagccc
gaccgctgcgccttatccggtaactatcgtcttgagtccaacccggtaagacacgacttatcgccactggcagcagccactggtaacaggat
tagcagagcgaggtatgtaggcggtgctacagagttcttgaagtggtggcctaactacggctacactagaaggacagtatttggtatctgcg
ctctgctgaagccagttaccttcggaaaaagagttggtagctcttgatccggcaaacaaaccaccgctggtagcggtggtttttttgtttgcaa
gcagcagattacgcgcagaaaaaaaggatctcaa
Ampicillin resistance gene (SEQ ID NO: 14):
ttaccaatgcttaatcagtgaggcacctatctcagcgatctgtctatttcgttcatccatagttgcctgactccccgtcgtgtagataactacgata
cgggagggcttaccatctggccccagtgctgcaatgataccgcgagacccacgctcaccggctccagatttatcagcaataaaccagcca
gccggaagggccgagcgcagaagtggtcctgcaactttatccgcctccatccagtctattaattgttgccgggaagctagagtaagtagttc
gccagttaatagtttgcgcaacgttgttgccattgctacaggcatcgtggtgtcacgctcgtcgtttggtatggcttcattcagctccggttccca
acgatcaaggcgagttacatgatcccccatgttgtgcaaaaaagcggttagctccttcggtcctccgatcgttgtcagaagtaagttggccgc
agtgttatcactcatggttatggcagcactgcataattctcttactgtcatgccatccgtaagatgcttttctgtgactggtgagtactcaaccaag
tcattctgagaatagtgtatgcggcgaccgagttgctcttgcccggcgtcaatacgggataataccgcgccacatagcagaactttaaaagtg
ctcatcattggaaaacgttcttcggggcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgcaaaaaagggaataagggcgacacg
gaaatgttgaatactcat
GS gene (SEQ ID NO: 15):
Atggccacctcagcaagttcccacttgaacaaaaacatcaagcaaatgtacttgtgcctgccccagggtgagaaagtccaagccatgtatat
ctgggttgatggtactggagaaggactgcgctgcaaaacccgcaccctggactgtgagcccaagtgtgtagaagagttacctgagtggaat
tttgatggctctagtacctttcagtctgagggctccaacagtgacatgtatctcagccctgttgccatgtttcgggaccccttccgcagagatcc
caacaagctggtgttctgtgaagttttcaagtacaaccggaagcctgcagagaccaatttaaggcactcgtgtaaacggataatggacatggt
gagcaaccagcacccctggtttggaatggaacaggagtatactctgatgggaacagatgggcacccttttggttggccttccaatggctttcc
tgggccccaaggtccgtattactgtggtgtgggcgcagacaaagcctatggcagggatatcgtggaggctcactaccgcgcctgcttgtat
gctggggtcaagattacaggaacaaatgctgaggtcatgcctgcccagtgggagttccaaataggaccctgtgaaggaatccgcatggga
gatcatctctgggtggcccgtttcatcttgcatcgagtatgtgaagactttggggtaatagcaacctttgaccccaagcccattcctgggaact
ggaatggtgcaggctgccataccaactttagcaccaaggccatgcgggaggagaatggtctgaagcacatcgaggaggccatcgagaaa
ctaagcaagcggcaccggtaccacattcgagcctacgatcccaaggggggcctggacaatgcccgtcgtctgactgggttccacgaaac
gtccaacatcaacgacttttctgctggtgtcgccaatcgcagtgccagcatccgcattccccggactgtcggccaggagaagaaaggttact
ttgaagaccgccgcccctctgccaattgtgacccctttgcagtgacagaagccatcgtccgcacatgccttctcaatgagactggcgacgag
cccttccaatacaaaaactaa
Neomycin resistance gene (neomycin phosphotransferase gene, SEQ ID NO: 16)
atgattgaacaagatggattgcacgcaggttctccggccgcttgggtggagaggctattcggctatgactgggcacaacagacaatcggct
gctctgatgccgccgtgttccggctgtcagcgcaggggcgcccggttctttttgtcaagaccgacctgtccggtgccctgaatgaactgcag
gacgaggcagcgcggctatcgtggctggccacgacgggcgttccttgcgcagctgtgctcgacgttgtcactgaagcgggaagggactg
gctgctattgggcgaagtgccggggcaggatctcctgtcatctcaccttgctcctgccgagaaagtatccatcatggctgatgcaatgcggc
ggctgcatacgcttgatccggctacctgcccattcgaccaccaagcgaaacatcgcatcgagcgagcacgtactcggatggaagccggtc
ttgtcgatcaggatgatctggacgaggagcatcaggggctcgcgccagccgaactgttcgccaggctcaaggcgcgcatgcccgacggc
gaggatctcgtcgtgacccatggcgatgcctgcttgccgaatatcatggtggaaaatggccgcttttctggattcatcgactgtggccggctg
ggtgtggcggaccgctatcaggacatagcgttggctacccgtgatattgctgaagaacttggcggcgaatgggctgaccgcttcctcgtgct
ttacggtatcgccgctcccgattcgcagcgcatcgccttctatcgccttcttgacgagttcttctga
Puromycin resistance gene (puromycin N-acetyl-transferase gene, SEQ ID NO: 17)
atgaccgagtacaagcccacggtgcgcctcgccacccgcgacgacgtcccccgggccgtacgcaccctcgccgccgcgttcgccgact
accccgccacgcgccacaccgtcgacccggaccgccacatcgagcgggtcaccgagctgcaagaactcttcctcacgcgcgtcgggct
cgacatcggcaaggtgtgggtcgcggacgacggcgccgcggtggcggtctggaccacgccggagagcgtcgaagcgggggcggtgt
tcgccgagatcggcccgcgcatggccgagttgagcggttcccggctggccgcgcagcaacagatggaaggcctcctggcgccgcacc
ggcccaaggagcccgcgtggttcctggccaccgtcggcgtctcgcccgaccaccagggcaagggtctgggcagcgccgtcgtgctccc
cggagtggaggcggccgagcgcgccggggtgcccgccttcctggagacctccgcgccccgcaacctccccttctacgagcggctcggc
ttcaccgtcaccgccgacgtcgaggtgcccgaaggaccgcgcacctggtgcatgacccgcaagcccggtgcc
Blasticidin resistance gene (SEQ ID NO: 18)
atggccaagcctttgtctcaagaagaatccaccctcattgaaagagcaacggctacaatcaacagcatccccatctctgaagactacagcgt
cgccagcgcagctctctctagcgacggccgcatcttcactggtgtcaatgtatatcattttactgggggaccttgtgcagaactcgtggtgctg
ggcactgctgctgctgcggcagctggcaacctgacttgtatcgtcgctatcggaaatgagaacaggggcatcttgagcccctgcggacggt
gccgacaggtgcttctcgatctgcatcctgggatcaaagccatagtgaaggacagtgatggacagccgacggcagttgggattcgtgaatt
gctgccctctggttatgtgtgggagggctaa
SV40 late polyA (Mol Cell Biol. 9(10):4248-58 (1989), GenBank: J02400.1, SEQ ID NO: 19):
cagacatgataagatacattgatgagtttggacaaaccacaactagaatgcagtgaaaaaaatgctttatttgtgaaatttgtgatgctattgcttt
atttgtaaccattataagctgcaataaacaagttaacaacaacaattgcattcattttatgtttcaggttcagggggaggtgtgggaggttttttaa
agcaagtaaaacctctacaaatgtggta
SV40 promoter (Nature 273(5658):113-20 (1978), Proc. Natl. Acad. Sci. USA 81 (1):23-27
(1984), GenBank: J02400.1, SEQ ID NO: 20):
tgcatctcaattagtcagcaaccatagtcccgcccctaactccgcccatcccgcccctaactccgcccagttccgcccattctccgccccatc
gctgactaattttttttatttatgcagaggccgaggccgcctcggcctctgagctattccagaagtagtgaggaggcttttttggaggcctaggc
ttttgcaaa
SV40 enhancer (SEQ ID NO: 21):
gctgtggaatgtgtgtcagttagggtgtggaaagtccccaggctccccagcaggcagaagtatgcaaagcatgcatctcaattagtcagca
accaggtgtggaaagtccccaggctccccagcaggcagaagtatgcaaagcatgcatctcaattagtcagcaaccatagtcccgccccta
actccgcccatcccgcccctaactccgcccagttccgcccattctccgctccatcg
pCLD-BDDE-2A (with mammalian selection marker A, SEQ ID NO: 22):
ccaatgatcttaagttaatcgaatttgcagcccgggactagctacttaagatccgaccggacgcgttctattaccacatttgtagaggttttactt
gctttaaaaaacctcccacatctccccctgaacctgaaacataaaatgaatgcaattgttgttgttaacttgtttattgcagcttataatggttacaa
ataaagcaatagcatcacaaatttcacaaataaagcatttttttcactgcattctagttgtggtttgtccaaactcatcaatgtatcttatgatgtgtg
atcagttatctatgcggccgcggtggcggcgtcgaccgagaggttttccgatccggtcgatgcggactcgctcaggtccctcggtggcgga
gtaccgttcggaggccgacgggtttccgatccaagagtactggaaagaccgcgaagagtttgtcctcaaccgcgagctgtggaaaaaaaa
gggacaggataagtatgacatcatcaaggaagcttgacaacaaaaagattgtcttttctgaccagatggacgcggccaccctcaaaggcat
caccgcgggccaggtgaatatcaaatcctcctcgtttttggaaactgacaatcttagcgcagaagtcatgcccgcttttgagagggagtactc
accccaacagctggccctcgcagacagcgatgcggaagaggatctgacggttcactaaacgagctctgcttatatagacctcccaccgtac
acgcctaccgcccatttgcgtcaatggggcggagttgttacgacattttggaaagtcccgttgattttggtgccaaaacaaactcccattgacg
tcaatggggtggagacttggaaatccccgtgagtcaaaccgctatccacgcccattgatgtactgccaaaaccgcatcaccatggtaatagc
gatgactaatacgtagatgtactgccaagtaggaaagtcccataaggtcatgtactgggcataatgccaggcgggccatttaccgtcattgac
gtcaataggggcgtacttggcatatgatacacttgatgtactgccaagtgggcagtttaccgtaaatactccacccattgacgtcaatggaaa
gtccctattggcgttactatgggaacatacgtcattattgacgtcaatgggcgggggtcgttgggcggtcagccaggcgggccatttaccgt
aagttatgtaacactgacacacattccacagctgcctcgcgcgtttcggtgatgacggtgaaaacctctgacacatgcagctcccggagacg
gtcacagcttgtctgtaagcggatgccgggagcagacaagcccgtcagggcgcgtcagcgggtgttggcgggtgtcggggcgcagcca
tgacccagtcacgtagcgatagcggagtgtatactggcttaactatgcggcatcagagcagattgtactgagagcgctattctgaacttttcttt
tgttcccttcccttctaccacaccctaattgtaatccattttaatttcctggtcacagtcctgtctctccttccattgtaccttgcccttttctaaagagc
gactgcaaagtatgtttgcgtaggtgaggatctaaaactttatgaggtacgaacatcacagaattactttgtaatttcagtttattgtaggcttggc
tttttggggagggtttacgtcttagacctcttagtgcttctttgtttcatggtgttctaacttcgaagcatctctgtagctttaatggattccattctga
aagctttgctctctttcttccccctcggctttctcttaggcaagagggctaactgtaaagtaaggcttactgccttgtgtttccaaatgtgtccgaa
gaggaagtgtcttctgtgaatcctgttatgcatgaataacaggaaatagaaagaaattcactttcattattataaaagtaatatgttcgtttaaaaa
attctaatgaagagctggagatgcaacccaggggtagagcacacactcagcatgcaggaggccctgggtccaatcttggaatctcctctca
gttaacctgatctctagctgattagtagtgagtgcaagcccactttcctcttctgcctcattgctcagtgataacagctgttaaactttgtcttattct
aaaactacctctgtgcaaatgctagcacaataatatatatcatatgcacatgattttttttttatcttgaaaagtaagtcagtatagctacaaagttca
cttggcattgtcaacatttcacaggcgtaatattcctcctctagtactgtcctcttcattctttgtgaccaagtttggagagagtgcacaaatgcca
gggaggtttgtgggaaggtttctcatgttctggtaaggcgagtaagaaaatagtctcatgcaggtgaaatgagtgctatgcagtatatattatac
cagagaacagcaaatgaccaaattcacactgaactagttcagtaaaattggctttgtcaaagctttccttgcttaaaatgtaattccctgtcatcc
tagttctggtctggattcttttcctggagtcttgacttccagattccctgtggacttttgtttgagtttcaagcttttgaaatatagaaacctatctaact
taacaaacttgggagagaaaagactccagaacaactgaaaacagaccaggctaaatgaatagactttattcctctcttcttacctgcagttttc
agatatgcagagttggagcggatcttagaggttgattcattcatgcctgaagaaaacacattttatagaccctgtgcccaagttcgtggtggac
atcaccctttatttactaattgcactacataacaggcattttagaagactgctccagtcagagaccccgccttagaggaatctgtaaaccctgaa
ctcctatcactcatgagcactagttatgtttggaatgccgtattaaaacaaaagttacatttctaaacttaaaattttctagcacagagacagtggg
agtagctaactttgatagacatttttctactaaaagtctttctaagtacataatcttctgtaagttggaaaacagcaaaatagaacgtctcctacgta
gttaatctttttgcataatttgcacatgtaggagttattagtatacgggtaagttttcactttttcccccaactggagtgtcttgtggctgggtttgaa
aaagggaacgggaggccgctggaggggattggtaaatgagataaaacaccactcattcaactcagtgactcagcatttaaattttccataaa
aggattaaaggaaaattaaacaaattcttaaagccaagactctggagaaacttgttggtgtgctttagttttcactgttatgactcatgaatttatg
cataaattagtacatttataaaaacatagcctttttagagttttctgtttggctaaagtgccattgttagcatttggaattacctttttatgtcttatattt
tttccaaataaaaataaatgtttctgctgtcttactactgaaactacgttgtgagcactttaaatttctcaaagcagtttcgcctgttatacttggcgctt
agtcatcgtcgtacacaacaggacctgattaagaaggctgtgctgcctctaagccgggctagattgtagccactagcaaccaggctgcaata
atttccctttgatgacatcatccactgtggaagaacccagttgcttcagccagtcgaactatacagttccaacctcatcaaatatggcatctccct
tgcctgctatagcagggggaggaaaaaatgccaccatctttttaatctagcaagcttctcttttcttcatctttttttttttcttttaaaaaaattctgat
catggatgcttcttccgatccctatttgccttatgacgggggaggagacaatatccccttgagggaattacataaaagaggtaagagcatccc
cttgctctgaatcctctgttggttgttgtgcatgcggctgggcggttctggggacaggctgtctgttgtcctcttgctgcaatgtgctgcttagttg
ccctgccttgttgctgtgggagaatgcgaccttcccagcagggctggccctccctgattgtttgctctgtgcagattagccctgcttcagatca
catagggctgcagactccatcttctgtgtgaaaatgctttcggtttgattgcagaaataagctgcctttacagccagctaaagtcctggtggttg
gttggcacctgcaaagtagtatttttgtacctctggaaacttatattttctttacacagcaatatcaagtgccggtatgccattctgttttggctgctg
ccaattaccatgtagactttgcaccacagagtaatagtaaaagctcctagctgcattttataacatttaaaaatagcaggaaagaagaattatttt
tgatttaacatgtttttgtcatttaacgtcttaactgattgacatactatattgtctgtctcgtgggtatcttgtacaacttgataggataaagcaattta
gtttttttttttttttttaaatacatccagaatgtaagtcgtcagtagttttcgaacagataagtaatggtgttaatcttttggcaggctttgccttggtct
ccttaaagctaattaggtgttacttaattaaactgctcttttgctcattttcttaaatttttttttttaaaagatagttggcatttgctgttctagaaataa
acttcaagaaacattctttagccagatgacttcatgtatgagccatgttagtttgaattatttgcttggtgttataaactttatggtttaataccaactttt
attatgtttacaaggtaaataaggaaaatttcaagtacattttgtatcctgagaacaaatttaagttccatagaatttaggaattacaatgtattcaac
agatacttacttgtcatactgtgcctgcaaaacaataattagactctgaacaggtgcaacaattttctgtagaattagacaagtcttcttttggcag
gtgttactaagtaggccatttcccaaggaacagggaatttgccaggcttttgtggtggagagaatagaatgaataaatgctgtggggagtaaa
gagcttgtcagaagatgattagttctgtggcaccaaaaccaagagatcagttttcctgtgagaagtaaaggaagcattgtagaaaaatagatg
tgttgaagtctaccggtggagttccgcgttacataacttacggtaaatggcccgcctggctgaccgcccaacgacccccgcccattgacgtc
aataatgacgtatgttcccatagtaacgccaatagggactttccattgacgtcaatgggtggagtatttacggtaaactgcccacttggcagta
catcaagtgtatcatatgccaagtacgccccctattgacgtcaatgacggtaaatggcccgcctggcattatgcccagtacatgaccttatgg
gactttcctacttggcagtacatctacgtattagtcatcgctattaccattgtgatgcggttttggcagtacatcaatgggcgtggatagcggtttg
actcacggggatttccaagtctccaccccattgacgtcaatgggagtttgttttggcaccaaaatcaacgggactttccaaaatgtcgtaacaa
ctccgccccattgacgcaaatgggcggtaggcgtgtacggtgggaggtctatataagcagagctcgtttagtgaaccgtcagatctacctctt
ccgcatcgctgtctgcgagggccagctgttggggtgagtactccctctcaaaagcgggcatgacttctgcgctaagattgtcagtttccaaaa
acgaggaggatttgatattcacctggcccgcggtgatgcctttgagggtggccgcgtccatctggtcagaaaagacaatctttttgttgtcaag
cttccttgatgatgtcatacttatcctgtcccttttttttccacagctcgcggttgaggacaaactcttcgcggtctttccagtactcttggatcgga
aacccgtcggcctccgaacggtactccgccaccgagggacctgagcgagtccgcatcgaccggatcggaaaacctcggatccgccgcc
accgaattcatagataactgatccagtgcccctaacgttactggccgaagccgcttggaataaggccggtgtgcgtttgtctatatgttattttc
caccatattgccgtcttttggcaatgtgagggcccggaaacctggccctgtcttcttgacgagcattcctaggggtctttcccctctcgccaaa
ggaatgcaaggtctgttgaatgtcgtgaaggaagcagttcctctggaagcttcttgaagacaaacaacgtctgtagcgaccctttgcaggca
gcggaaccccccacctggcgacaggtgcctctgcggccaaaagccacgtgtataagatacacctgcaaaggcggcacaaccccagtgc
cacgttgtgagttggatagttgtggaaagagtcaaatggctctcctcaagcgtattcaacaaggggctgaaggatgcccagaaggtacccc
attgtatgggatctgatctggggcctcggtgcacatgctttacatgtgtttagtcgaggttaaaaaacgtctaggccccccgaaccacgggga
cgtggttttcctttgaaaaacacgatgataagcttgccacaacccacaaggagacgaccttccatgattgaacaagatggattgcacgcaggt
tctccggccgcttgggtggagaggctattcggctatgactgggcacaacagacaatcggctgctctgatgccgccgtgttccggctgtcag
cgcaggggcgcccggttctttttgtcaagaccgacctgtccggtgccctgaatgaactgcaggacgaggcagcgcggctatcgtggctgg
ccacgacgggcgttccttgcgcagctgtgctcgacgttgtcactgaagcgggaagggactggctgctattgggcgaagtgccggggcag
gatctcctgtcatctcaccttgctcctgccgagaaagtatccatcatggctgatgcaatgcggcggctgcatacgcttgatccggctacctgcc
cattcgaccaccaagcgaaacatcgcatcgagcgagcacgtactcggatggaagccggtcttgtcgatcaggatgatctggacgaggagc
atcaggggctcgcgccagccgaactgttcgccaggctcaaggcgcgcatgcccgacggcgaggatctcgtcgtgacccatggcgatgc
ctgcttgccgaatatcatggtggaaaatggccgcttttctggattcatcgactgtggccggctgggtgtggcggaccgctatcaggacatagc
gttggctacccgtgatattgctgaagaacttggcggcgaatgggctgaccgcttcctcgtgctttacggtatcgccgctcccgattcgcagcg
catcgccttctatcgccttcttgacgagttcttctgatctagatccccctcgctttcttgctgtccaatttctattaaaggttcctttgttccctaagtcc
aactactaaactgggggatattatgaagggccttgagcatctggattctgcctaataaaaaacatttattttcattgcaatgatgtatttaaattattt
ctgaatattttactaaaaagggaatgtgggaggtcagtgcatttaaaacataaagaaatgaagagggggatcttcgcgatactgcatcgatta
gacaccgcggtggagctccagcttttgttccctttagtgagggttaattagttcttaatacgactcactatagggcgaattggctaccgggccg
cccatcgagggtatcataagcttttaaatcgatagatgcgatatcggaaagaacatgtgagcaaaaggccagcaaaaggccaggaaccgt
aaaaaggccgcgttgctggcgtttttccataggctccgcccccctgacgagcatcacaaaaatcgacgctcaagtcagaggtggcgaaac
ccgacaggactataaagataccaggcgtttccccctggaagctccctcgtgcgctctcctgttccgaccctgccgcttaccggatacctgtcc
gcctttctcccttcgggaagcgtggcgctttctcatagctcacgctgtaggtatctcagttcggtgtaggtcgttcgctccaagctgggctgtgt
gcacgaaccccccgttcagcccgaccgctgcgccttatccggtaactatcgtcttgagtccaacccggtaagacacgacttatcgccactgg
cagcagccactggtaacaggattagcagagcgaggtatgtaggcggtgctacagagttcttgaagtggtggcctaactacggctacactag
aagaacagtatttggtatctgcgctctgctgaagccagttaccttcggaaaaagagttggtagctcttgatccggcaaacaaaccaccgctgg
tagcggtggtttttttgtttgcaagcagcagattacgcgcagaaaaaaaggatctcaagaagatcctttgatcttttctacggggtctgacgctc
agtggaacgaaaactcacgttaagggattttggtcatgagattatcaaaaaggatcttcacctagatccttttaaattaaaaatgaagttttaaat
caatctaaagtatatatgagtaaacttggtctgacagttaccaatgcttaatcagtgaggcacctatctcagcgatctgtctatttcgttcatccat
agttgcctgactccccgtcgtgtagataactacgatacgggagggcttaccatctggccccagtgctgcaatgataccgcgagacccacgc
tcaccggctccagatttatcagcaataaaccagccagccggaagggccgagcgcagaagtggtcctgcaactttatccgcctccatccagt
ctattaattgttgccgggaagctagagtaagtagttcgccagttaatagtttgcgcaacgttgttgccattgctacaggcatcgtggtgtcacgc
tcgtcgtttggtatggcttcattcagctccggttcccaacgatcaaggcgagttacatgatcccccatgttgtgcaaaaaagcggttagctcctt
cggtcctccgatcgttgtcagaagtaagttggccgcagtgttatcactcatggttatggcagcactgcataattctcttactgtcatgccatccgt
aagatgcttttctgtgactggtgagtactcaaccaagtcattctgagaatagtgtatgcggcgaccgagttgctcttgcccggcgtcaatacgg
gataataccgcgccacatagcagaactttaaaagtgctcatcattggaaaacgttcttcggggcgaaaactctcaaggatcttaccgctgttg
agatccagttcgatgtaacccactcgtgcacccaactgatcttcagcatcttttactttcaccagcgtttctgggtgagcaaaaacaggaaggc
aaaatgccgcaaaaaagggaataagggcgacacggaaatgttgaatactcatactcttcctttttcaatattattgaagcatttatcagggttatt
gtctcatgagcggatacatatttgaatgtatttagaaaaataaacaaataggggttccgcgcacatttccccgaaaagtgccacctgacgtc
pCLD-BDDE-2B (with mammalian selection marker B, SEQ ID NO: 23):
ccaatgatcttaagttaatcgaatttgcagcccgggactagctacttaagatccgaccggacgcgttctattaccacatttgtagaggttttactt
gctttaaaaaacctcccacatctccccctgaacctgaaacataaaatgaatgcaattgttgttgttaacttgtttattgcagcttataatggttacaa
ataaagcaatagcatcacaaatttcacaaataaagcatttttttcactgcattctagttgtggtttgtccaaactcatcaatgtatcttatgatgtgtg
atcagttatctatgcggccgcggtggcggcgtcgaccgagaggttttccgatccggtcgatgcggactcgctcaggtccctcggtggcgga
gtaccgttcggaggccgacgggtttccgatccaagagtactggaaagaccgcgaagagtttgtcctcaaccgcgagctgtggaaaaaaaa
gggacaggataagtatgacatcatcaaggaagcttgacaacaaaaagattgtcttttctgaccagatggacgcggccaccctcaaaggcat
caccgcgggccaggtgaatatcaaatcctcctcgtttttggaaactgacaatcttagcgcagaagtcatgcccgcttttgagagggagtactc
accccaacagctggccctcgcagacagcgatgcggaagaggatctgacggttcactaaacgagctctgcttatatagacctcccaccgtac
acgcctaccgcccatttgcgtcaatggggcggagttgttacgacattttggaaagtcccgttgattttggtgccaaaacaaactcccattgacg
tcaatggggtggagacttggaaatccccgtgagtcaaaccgctatccacgcccattgatgtactgccaaaaccgcatcaccatggtaatagc
gatgactaatacgtagatgtactgccaagtaggaaagtcccataaggtcatgtactgggcataatgccaggcgggccatttaccgtcattgac
gtcaataggggcgtacttggcatatgatacacttgatgtactgccaagtgggcagtttaccgtaaatactccacccattgacgtcaatggaaa
gtccctattggcgttactatgggaacatacgtcattattgacgtcaatgggcgggggtcgttgggcggtcagccaggcgggccatttaccgt
aagttatgtaacactgacacacattccacagctgcctcgcgcgtttcggtgatgacggtgaaaacctctgacacatgcagctcccggagacg
gtcacagcttgtctgtaagcggatgccgggagcagacaagcccgtcagggcgcgtcagcgggtgttggcgggtgtcggggcgcagcca
tgacccagtcacgtagcgatagcggagtgtatactggcttaactatgcggcatcagagcagattgtactgagagcgctattctgaacttttcttt
tgttcccttcccttctaccacaccctaattgtaatccattttaatttcctggtcacagtcctgtctctccttccattgtaccttgcccttttctaaagagc
gactgcaaagtatgtttgcgtaggtgaggatctaaaactttatgaggtacgaacatcacagaattactttgtaatttcagtttattgtaggcttggc
tttttggggagggtttacgtcttagacctcttagtgcttctttgtttcatggtgttctaacttcgaagcatctctgtagctttaatggattccttttctga
aagctttgctctctttcttccccctcggctttctcttaggcaagagggctaactgtaaagtaaggcttactgccttgtgtttccaaatgtgtccgaa
gaggaagtgtcttctgtgaatcctgttatgcatgaataacaggaaatagaaagaaattcactttcattattataaaagtaatatgttcgtttaaaaa
attctaatgaagagctggagatgcaacccaggggtagagcacacactcagcatgcaggaggccctgggtccaatcttggaatctcctctca
gttaacctgatctctagctgattagtagtgagtgcaagcccactttcctcttctgcctcattgctcagtgataacagctgttaaactttgtcttattct
aaaactacctctgtgcaaatgctagcacaataatatatatcatatgcacatgttttttttttttatcttgaaaagtaagtcagtatagctacaaagttca
cttggcattgtcaacatttcacaggcgtaatattcctcctctagtactgtcctcttcattctttgtgaccaagtttggagagagtgcacaaatgcca
gggaggtttgtgggaaggtttctcatgttctggtaaggcgagtaagaaaatagtctcatgcaggtgaaatgagtgctatgcagtatatattatac
cagagaacagcaaatgaccaaattcacactgaactagttcagtaaaattggctttgtcaaagctttccttgcttaaaatgtaattccctgtcatcc
tagttctggtctggattcttttcctggagtcttgacttccagattccctgtggacttttgtttgagtttcaagcttttgaaatatagaaacctatctaact
taacaaacttgggagagaaaagactccagaacaactgaaaacagaccaggctaaatgaatagactttattcctctcttcttacctgcagttttc
agatatgcagagttggagcggatcttagaggttgattcattcatgcctgaagaaaacacattttatagaccctgtgcccaagttcgtggtggac
atcaccctttatttactaattgcactacataacaggcattttagaagactgctccagtcagagaccccgccttagaggaatctgtaaaccctgaa
ctcctatcactcatgagcactagttatgtttggaatgccgtattaaaacaaaagttacatttctaaacttaaaattttctagcacagagacagtggg
agtagctaactttgatagacatttttctactaaaagtctttctaagtacataatcttctgtaagttggaaaacagcaaaatagaacgtctcctacgta
gttaatctttttgcataatttgcacatgtaggagttattagtatacgggtaagttttcactttttcccccaactggagtgtcttgtggctgggtttgaa
aaagggaacgggaggccgctggaggggattggtaaatgagataaaacaccactcattcaactcagtgactcagcatttaaattttccataaa
aggattaaaggaaaattaaacaaattcttaaagccaagactctggagaaacttgttggtgtgctttagttttcactgttatgactcatgaatttatg
cataaattagtacatttataaaaacatagcctttttagagttttctgtttggctaaagtgccattgttagcatttggaattacctttttatgtcttatattt
tttccaaataaaaataaatgtttctgctgtcttactactgaaactacgttgtgagcactttaaatttctcaaagcagtttcgcctgttatacttggcgctt
agtcatcgtcgtacacaacaggacctgattaagaaggctgtgctgcctctaagccgggctagattgtagccactagcaaccaggctgcaata
atttccctttgatgacatcatccactgtggaagaacccagttgcttcagccagtcgaactatacagttccaacctcatcaaatatggcatctccct
tgcctgctatagcagggggaggaaaaaatgccaccatctttttaatctagcaagcttctcttttcttcatctttttttttttcttttaaaaaaattctgat
catggatgcttcttccgatccctatttgccttatgacgggggaggagacaatatccccttgagggaattacataaaagaggtaagagcatccc
cttgctctgaatcctctgttggttgttgtgcatgcggctgggcggttctggggacaggctgtctgttgtcctcttgctgcaatgtgctgcttagttg
ccctgccttgttgctgtgggagaatgcgaccttcccagcagggctggccctccctgattgtttgctctgtgcagattagccctgcttcagatca
catagggctgcagactccatcttctgtgtgaaaatgctttcggtttgattgcagaaataagctgcctttacagccagctaaagtcctggtggttg
gttggcacctgcaaagtagtatttttgtacctctggaaacttatattttctttacacagcaatatcaagtgccggtatgccattctgttttggctgctg
ccaattaccatgtagactttgcaccacagagtaatagtaaaagctcctagctgcattttataacatttaaaaatagcaggaaagaagaattatttt
tgatttaacatgtttttgtcatttaacgtcttaactgattgacatactatattgtctgtctcgtgggtatcttgtacaacttgataggataaagcaattta
gtttttttttttttttttaaatacatccagaatgtaagtcgtcagtagttttcgaacagataagtaatggtgttaatcttttggcaggctttgccttggtct
ccttaaagctaattaggtgttacttaattaaactgctcttttgctcattttcttaaattatttttttaaaagatagttggcatttgctgttctagaaataaa
cttcaagaaacattctttagccagatgacttcatgtatgagccatgttagtttgaattatttgcttggtgttataaactttatggtttaataccaactttta
ttatgtttacaaggtaaataaggaaaatttcaagtacattttgtatcctgagaacaaatttaagttccatagaatttaggaattacaatgtattcaac
agatacttacttgtcatactgtgcctgcaaaacaataattagactctgaacaggtgcaacaattttctgtagaattagacaagtcttcttttggcag
gtgttactaagtaggccatttcccaaggaacagggaatttgccaggcttttgtggtggagagaatagaatgaataaatgctgtggggagtaaa
gagcttgtcagaagatgattagttctgtggcaccaaaaccaagagatcagttttcctgtgagaagtaaaggaagcattgtagaaaaatagatg
tgttgaagtctaccggtggagttccgcgttacataacttacggtaaatggcccgcctggctgaccgcccaacgacccccgcccattgacgtc
aataatgacgtatgttcccatagtaacgccaatagggactttccattgacgtcaatgggtggagtatttacggtaaactgcccacttggcagta
catcaagtgtatcatatgccaagtacgccccctattgacgtcaatgacggtaaatggcccgcctggcattatgcccagtacatgaccttatgg
gactttcctacttggcagtacatctacgtattagtcatcgctattaccattgtgatgcggttttggcagtacatcaatgggcgtggatagcggtttg
actcacggggatttccaagtctccaccccattgacgtcaatgggagtttgttttggcaccaaaatcaacgggactttccaaaatgtcgtaacaa
ctccgccccattgacgcaaatgggcggtaggcgtgtacggtgggaggtctatataagcagagctcgtttagtgaaccgtcagatctacctctt
ccgcatcgctgtctgcgagggccagctgttggggtgagtactccctctcaaaagcgggcatgacttctgcgctaagattgtcagtttccaaaa
acgaggaggatttgatattcacctggcccgcggtgatgcctttgagggtggccgcgtccatctggtcagaaaagacaatctttttgttgtcaag
cttccttgatgatgtcatacttatcctgtcccttttttttccacagctcgcggttgaggacaaactcttcgcggtctttccagtactcttggatcgga
aacccgtcggcctccgaacggtactccgccaccgagggacctgagcgagtccgcatcgaccggatcggaaaacctcggatccgccgcc
accgaattcatagataactgatccagtgcccctaacgttactggccgaagccgcttggaataaggccggtgtgcgtttgtctatatgttattttc
caccatattgccgtcttttggcaatgtgagggcccggaaacctggccctgtcttcttgacgagcattcctaggggtctttcccctctcgccaaa
ggaatgcaaggtctgttgaatgtcgtgaaggaagcagttcctctggaagcttcttgaagacaaacaacgtctgtagcgaccctttgcaggca
gcggaaccccccacctggcgacaggtgcctctgcggccaaaagccacgtgtataagatacacctgcaaaggcggcacaaccccagtgc
cacgttgtgagttggatagttgtggaaagagtcaaatggctctcctcaagcgtattcaacaaggggctgaaggatgcccagaaggtacccc
attgtatgggatctgatctggggcctcggtgcacatgctttacatgtgtttagtcgaggttaaaaaacgtctaggccccccgaaccacgggga
cgtggttttcctttgaaaaacacgatgataagcttgccacaacccacaaggagacgaccttccatgaccgagtacaagcccacggtgcgcct
cgccacccgcgacgacgtcccccgggccgtacgcaccctcgccgccgcgttcgccgactaccccgccacgcgccacaccgtcgaccc
ggaccgccacatcgagcgggtcaccgagctgcaagaactcttcctcacgcgcgtcgggctcgacatcggcaaggtgtgggtcgcggac
gacggcgccgcggtggcggtctggaccacgccggagagcgtcgaagcgggggcggtgttcgccgagatcggcccgcgcatggccga
gttgagcggttcccggctggccgcgcagcaacagatggaaggcctcctggcgccgcaccggcccaaggagcccgcgtggttcctggcc
accgtcggcgtctcgcccgaccaccagggcaagggtctgggcagcgccgtcgtgctccccggagtggaggcggccgagcgcgccgg
ggtgcccgccttcctggagacctccgcgccccgcaacctccccttctacgagcggctcggcttcaccgtcaccgccgacgtcgaggtgcc
cgaaggaccgcgcacctggtgcatgacccgcaagcccggtgcctctagatccccctcgctttcttgctgtccaatttctattaaaggttccttt
gttccctaagtccaactactaaactgggggatattatgaagggccttgagcatctggattctgcctaataaaaaacatttattttcattgcaatgat
gtatttaaattatttctgaatattttactaaaaagggaatgtgggaggtcagtgcatttaaaacataaagaaatgaagagggggatcttcgcgat
actgcatcgattagacaccgcggtggagctccagcttttgttccctttagtgagggttaattagttcttaatacgactcactatagggcgaattgg
ctaccgggccgcccatcgagggtatcataagcttttaaatcgatagatgcgatatcggaaagaacatgtgagcaaaaggccagcaaaagg
ccaggaaccgtaaaaaggccgcgttgctggcgtttttccataggctccgcccccctgacgagcatcacaaaaatcgacgctcaagtcagag
gtggcgaaacccgacaggactataaagataccaggcgtttccccctggaagctccctcgtgcgctctcctgttccgaccctgccgcttaccg
gatacctgtccgcctttctcccttcgggaagcgtggcgctttctcatagctcacgctgtaggtatctcagttcggtgtaggtcgttcgctccaag
ctgggctgtgtgcacgaaccccccgttcagcccgaccgctgcgccttatccggtaactatcgtcttgagtccaacccggtaagacacgactt
atcgccactggcagcagccactggtaacaggattagcagagcgaggtatgtaggcggtgctacagagttcttgaagtggtggcctaactac
ggctacactagaagaacagtatttggtatctgcgctctgctgaagccagttaccttcggaaaaagagttggtagctcttgatccggcaaacaa
accaccgctggtagcggtggtttttttgtttgcaagcagcagattacgcgcagaaaaaaaggatctcaagaagatcctttgatcttttctacgg
ggtctgacgctcagtggaacgaaaactcacgttaagggattttggtcatgagattatcaaaaaggatcttcacctagatccttttaaattaaaaa
tgaagttttaaatcaatctaaagtatatatgagtaaacttggtctgacagttaccaatgcttaatcagtgaggcacctatctcagcgatctgtctatt
tcgttcatccatagttgcctgactccccgtcgtgtagataactacgatacgggagggcttaccatctggccccagtgctgcaatgataccgcg
agacccacgctcaccggctccagatttatcagcaataaaccagccagccggaagggccgagcgcagaagtggtcctgcaactttatccgc
ctccatccagtctattaattgttgccgggaagctagagtaagtagttcgccagttaatagtttgcgcaacgttgttgccattgctacaggcatcgt
ggtgtcacgctcgtcgtttggtatggcttcattcagctccggttcccaacgatcaaggcgagttacatgatcccccatgttgtgcaaaaaagcg
gttagctccttcggtcctccgatcgttgtcagaagtaagttggccgcagtgttatcactcatggttatggcagcactgcataattctcttactgtca
tgccatccgtaagatgcttttctgtgactggtgagtactcaaccaagtcattctgagaatagtgtatgcggcgaccgagttgctcttgcccggc
gtcaatacgggataataccgcgccacatagcagaactttaaaagtgctcatcattggaaaacgttcttcggggcgaaaactctcaaggatctt
accgctgttgagatccagttcgatgtaacccactcgtgcacccaactgatcttcagcatcttttactttcaccagcgtttctgggtgagcaaaaa
caggaaggcaaaatgccgcaaaaaagggaataagggcgacacggaaatgttgaatactcatactcttcctttttcaatattattgaagcattta
tcagggttattgtctcatgagcggatacatatttgaatgtatttagaaaaataaacaaataggggttccgcgcacatttccccgaaaagtgccac
ctgacgtc
pCLD-SE (with mammalian selection marker C, SEQ ID NO: 24):
tcgaatttgcagcccgggactagctacttaagatccgaccggacgcgtactgagagcgctattctgaacttttcttttgttcccttcccttctacc
acaccctaattgtaatccattttaatttcctggtcacagtcctgtctctccttccattgtaccttgcccttttctaaagagcgactgcaaagtatgttt
gcgtaggtgaggatctaaaactttatgaggtacgaacatcacagaattactttgtaatttcagtttattgtaggcttggctttttggggagggttta
cgtcttagacctcttagtgcttctttgtttcatggtgttctaacttcgaagcatctctgtagctttaatggattccttttctgaaagctttgctctctttct
tccccctcggctttctcttaggcaagagggctaactgtaaagtaaggcttactgccttgtgtttccaaatgtgtccgaagaggaagtgtcttctgt
gaatcctgttatgcatgaataacaggaaatagaaagaaattcactttcattattataaaagtaatatgttcgtttaaaaaattctaatgaagagctg
gagatgcaacccaggggtagagcacacactcagcatgcaggaggccctgggtccaatcttggaatctcctctcagttaacctgatctctagc
tgattagtagtgagtgcaagcccactttcctcttctgcctcattgctcagtgataacagctgttaaactttgtcttattctaaaactacctctgtgca
aatgctagcacaataatatatatcatatgcacatgattttttttttatcttgaaaagtaagtcagtatagctacaaagttcacttggcattgtcaacatt
tcacaggcgtaatattcctcctctagtactgtcctcttcattctttgtgaccaagtttggagagagtgcacaaatgccagggaggtttgtgggaa
ggtttctcatgttctggtaaggcgagtaagaaaatagtctcatgcaggtgaaatgagtgctatgcagtatatattataccagagaacagcaaat
gaccaaattcacactgaactagttcagtaaaattggctttgtcaaagctttccttgcttaaaatgtaattccctgtcatcctagttctggtctggatt
cttttcctggagtcttgacttccagattccctgtggacttttgtttgagtttcaagcttttgaaatatagaaacctatctaacttaacaaacttgggag
agaaaagactccagaacaactgaaaacagaccaggctaaatgaatagactttattcctctcttcttacctgcagttttcagatatgcagagttgg
agcggatcttagaggttgattcattcatgcctgaagaaaacacattttatagaccctgtgcccaagttcgtggtggacatcaccctttatttacta
attgcactacataacaggcattttagaagactgctccagtcagagaccccgccttagaggaatctgtaaaccctgaactcctatcactcatga
gcactagttatgtttggaatgccgtattaaaacaaaagttacatttctaaacttaaaattttctagcacagagacagtgggagtagctaactttgat
agacatttttctactaaaagtctttctaagtacataatcttctgtaagttggaaaacagcaaaatagaacgtctcctacgtagttaatctttttgcata
atttgcacatgtaggagttattagtatacgggtaagttttcactttttcccccaactggagtgtcttgtggctgggtttgaaaaagggaacggga
ggccgctggaggggattggtaaatgagataaaacaccactcattcaactcagtgactcagcatttaaattttccataaaaggattaaaggaaa
attaaacaaattcttaaagccaagactctggagaaacttgttggtgtgctttagttttcactgttatgactcatgaatttatgcataaattagtacattt
ataaaaacatagcctttttagagttttctgtttggctaaagtgccattgttagcatttggaattacctttttatgtcttatattttttccaaataaaaataa
atgtttctgctgtcttactactgaaactacgttgtgagcactttaaatttctcaaagcagtttcgcctgttatacttggcgcttagtcatcgtcgtaca
caacaggacctgattaagaaggctgtgctgcctctaagccgggctagattgtagccactagcaaccaggctgcaataatttccctttgatgac
atcatccactgtggaagaacccagttgcttcagccagtcgaactatacagttccaacctcatcaaatatggcatctcccttgcctgctatagca
gggggaggaaaaaatgccaccatctttttaatctagcaagcttctcttttcttcatctttttttttttcttttaaaaaaattctgatcatggatgcttcttc
cgatccctatttgccttatgacgggggaggagacaatatccccttgagggaattacataaaagaggtaagagcatccccttgctctgaatcct
ctgttggttgttgtgcatgcggctgggcggttctggggacaggctgtctgttgtcctcttgctgcaatgtgctgcttagttgccctgccttgttgct
gtgggagaatgcgaccttcccagcagggctggccctccctgattgtttgctctgtgcagattagccctgcttcagatcacatagggctgcaga
ctccatcttctgtgtgaaaatgctttcggtttgattgcagaaataagctgcctttacagccagctaaagtcctggtggttggttggcacctgcaaa
gtagtatttttgtacctctggaaacttatattttctttacacagcaatatcaagtgccggtatgccattctgttttggctgctgccaattaccatgtag
actttgcaccacagagtaatagtaaaagctcctagctgcattttataacatttaaaaatagcaggaaagaagaattatttttgatttaacatgttttt
gtcatttaacgtcttaactgattgacatactatattgtctgtctcgtgggtatcttgtacaacttgataggataaagcaatttagttttttttttttttttt
aaatacatccagaatgtaagtcgtcagtagttttcgaacagataagtaatggtgttaatcttttggcaggctttgccttggtctccttaaagctaatta
ggtgttacttaattaaactgctcttttgctcattttcttaaattatttttttaaaagatagttggcatttgctgttctagaaataaacttcaagaaacattc
tttagccagatgacttcatgtatgagccatgttagtttgaattatttgcttggtgttataaactttatggtttaataccaacttttattatgtttacaaggt
aaataaggaaaatttcaagtacattttgtatcctgagaacaaatttaagttccatagaatttaggaattacaatgtattcaacagatacttacttgtca
tactgtgcctgcaaaacaataattagactctgaacaggtgcaacaattttctgtagaattagacaagtcttcttttggcaggtgttactaagtagg
ccatttcccaaggaacagggaatttgccaggcttttgtggtggagagaatagaatgaataaatgctgtggggagtaaagagcttgtcagaag
atgattagttctgtggcaccaaaaccaagagatcagttttcctgtgagaagtaaaggaagcattgtagaaaaatagatgtgttgaagtctaccg
gtggagttccgcgttacataacttacggtaaatggcccgcctggctgaccgcccaacgacccccgcccattgacgtcaataatgacgtatgt
tcccatagtaacgccaatagggactttccattgacgtcaatgggtggagtatttacggtaaactgcccacttggcagtacatcaagtgtatcat
atgccaagtacgccccctattgacgtcaatgacggtaaatggcccgcctggcattatgcccagtacatgaccttatgggactttcctacttggc
agtacatctacgtattagtcatcgctattaccattgtgatgcggttttggcagtacatcaatgggcgtggatagcggtttgactcacggggatttc
caagtctccaccccattgacgtcaatgggagtttgttttggcaccaaaatcaacgggactttccaaaatgtcgtaacaactccgccccattgac
gcaaatgggcggtaggcgtgtacggtgggaggtctatataagcagagctcgtttagtgaaccgtcagatctacctcttccgcatcgctgtctg
cgagggccagctgttggggtgagtactccctctcaaaagcgggcatgacttctgcgctaagattgtcagtttccaaaaacgaggaggatttg
atattcacctggcccgcggtgatgcctttgagggtggccgcgtccatctggtcagaaaagacaatctttttgttgtcaagcttccttgatgatgt
catacttatcctgtcccttttttttccacagctcgcggttgaggacaaactcttcgcggtctttccagtactcttggatcggaaacccgtcggcctc
cgaacggtactccgccaccgagggacctgagcgagtccgcatcgaccggatcggaaaacctcggatccgaattcatagataactgatcca
gtgcccctaacgttactggccgaagccgcttggaataaggccggtgtgcgtttgtctatatgttattttccaccatattgccgtcttttggcaatgt
gagggcccggaaacctggccctgtcttcttgacgagcattcctaggggtctttcccctctcgccaaaggaatgcaaggtctgttgaatgtcgt
gaaggaagcagttcctctggaagcttcttgaagacaaacaacgtctgtagcgaccctttgcaggcagcggaaccccccacctggcgacag
gtgcctctgcggccaaaagccacgtgtataagatacacctgcaaaggcggcacaaccccagtgccacgttgtgagttggatagttgtggaa
agagtcaaatggctctcctcaagcgtattcaacaaggggctgaaggatgcccagaaggtaccccattgtatgggatctgatctggggcctc
ggtgcacatgctttacatgtgtttagtcgaggttaaaaaacgtctaggccccccgaaccacggggacgtggttttcctttgaaaaacacgatg
ataatatggccacaaccatggccacctcagcaagttcccacttgaacaaaaacatcaagcaaatgtacttgtgcctgccccagggtgagaaa
gtccaagccatgtatatctgggttgatggtactggagaaggactgcgctgcaaaacccgcaccctggactgtgagcccaagtgtgtagaag
agttacctgagtggaattttgatggctctagtacctttcagtctgagggctccaacagtgacatgtatctcagccctgttgccatgtttcgggacc
ccttccgcagagatcccaacaagctggtgttctgtgaagttttcaagtacaaccggaagcctgcagagaccaatttaaggcactcgtgtaaac
ggataatggacatggtgagcaaccagcacccctggtttggaatggaacaggagtatactctgatgggaacagatgggcacccttttggttg
gccttccaatggctttcctgggccccaaggtccgtattactgtggtgtgggcgcagacaaagcctatggcagggatatcgtggaggctcact
accgcgcctgcttgtatgctggggtcaagattacaggaacaaatgctgaggtcatgcctgcccagtgggagttccaaataggaccctgtga
aggaatccgcatgggagatcatctctgggtggcccgtttcatcttgcatcgagtatgtgaagactttggggtaatagcaacctttgaccccaa
gcccattcctgggaactggaatggtgcaggctgccataccaactttagcaccaaggccatgcgggaggagaatggtctgaagcacatcga
ggaggccatcgagaaactaagcaagcggcaccggtaccacattcgagcctacgatcccaaggggggcctggacaatgcccgtcgtctg
actgggttccacgaaacgtccaacatcaacgacttttctgctggtgtcgccaatcgcagtgccagcatccgcattccccggactgtcggcca
ggagaagaaaggttactttgaagaccgccgcccctctgccaattgtgacccctttgcagtgacagaagccatcgtccgcacatgccttctca
atgagactggcgacgagcccttccaatacaaaaactaatctagatccccctcgctttcttgctgtccaatttctattaaaggttcctttgttcccta
agtccaactactaaactgggggatattatgaagggccttgagcatctggattctgcctaataaaaaacatttattttcattgcaatgatgtatttaa
attatttctgaatattttactaaaaagggaatgtgggaggtcagtgcatttaaaacataaagaaatgaagagggggatcttcgcgatactgcatc
gattagacaccgcggtggagctccagcttttgttccctttagtgagggttaattagttcttaatacgactcactatagggcgaattggctaccgg
gccgcccatcgagggtatcataagcttttaaatcgatagatgcgatcctgcaggtctccctatagtgagtcgtattaatttcgataagccagctg
cattaatgaatcggccaacgcgcggggagaggcggtttgcgtattgggcgctcttccgcttcctcgctcactgactcgctgcgctcggtcgtt
cggctgcggcgagcggtatcagctcactcaaaggcggtaatacggttatccacagaatcaggggataacgcaggaaagaacatgtgagc
aaaaggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctcctgtt
ccgaccctgccgcttaccggatacctgtccgcctttctcccttcgggaagcgtggcgctttctcatagctcacgctgtaggtatctcagttcggt
gtaggtcgttcgctccaagctgggctgtgtgcacgaaccccccgttcagcccgaccgctgcgccttatccggtaactatcgtcttgagtccaa
cccggtaagacacgacttatcgccactggcagcagccactggtaacaggattagcagagcgaggtatgtaggcggtgctacagagttcttg
aagtggtggcctaactacggctacactagaaggacagtatttggtatctgcgctctgctgaagccagttaccttcggaaaaagagttggtagc
tcttgatccggcaaacaaaccaccgctggtagcggtggtttttttgtttgcaagcagcagattacgcgcagaaaaaaaggatctcaagaagat
cctttgatcttttctacggggtctgacgctcagtggaacgaaaactcacgttaagggattttggtcatgagattatcaaaaaggatcttcaccta
gatccttttaaattaaaaatgaagttttaaatcaatctaaagtatatatgagtaaacttggtctgacagttaccaatgcttaatcagtgaggcacct
atctcagcgatctgtctatttcgttcatccatagttgcctgactccccgtcgtgtagataactacgatacgggagggcttaccatctggccccag
tgctgcaatgataccgcgagacccacgctcaccggctccagatttatcagcaataaaccagccagccggaagggccgagcgcagaagtg
gtcctgcaactttatccgcctccatccagtctattaattgttgccgggaagctagagtaagtagttcgccagttaatagtttgcgcaacgttgttg
ccattgctacaggcatcgtggtgtcacgctcgtcgtttggtatggcttcattcagctccggttcccaacgatcaaggcgagttacatgatcccc
catgttgtgcaaaaaagcggttagctccttcggtcctccgatcgttgtcagaagtaagttggccgcagtgttatcactcatggttatggcagca
ctgcataattctcttactgtcatgccatccgtaagatgcttttctgtgactggtgagtactcaaccaagtcattctgagaatagtgtatgcggcga
ccgagttgctcttgcccggcgtcaatacgggataataccgcgccacatagcagaactttaaaagtgctcatcattggaaaacgttcttcgggg
cgaaaactctcaaggatcttaccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatactcatactcttcctttt
tcaatattattgaagcatttatcagggttattgtctcatgagcggatacatatttgaatgtatttagaaaaataaacaaataggggttccgcgcaca
tttccccgaaaagtgccacctgacgtctaagaaaccattattatcatgacattaacctataaaaataggcgtatcacgaggcctttcgtctcgc
gcgtttcggtgatgacggtgaaaacctctgacacatgcagctcccggagacggtcacagcttgtctgtaagcggatgccgggagcagaca
agcccgtcagggcgcgtcagcgggtgttggcgggtgtcggggctggcttaactatgcggcatcagagcagattgtactgagagtgcacca
tatcgacgctctcccttatgcgactcctgcattaggaagcagcccagtagtaggttgaggccgttgagcaccgccgccgcaaggaatggtg
catgcaaggagatggcgcccaacagtcccccggccacggggcctgccaccatacccacgccgaaacaagcgctcatgagcccgaagt
ggcgagcccgatcttccccatcggtgatgtcggcgatataggcgccagcaaccgcacctgtggcgccggtgatgccggccacgatgcgt
ccggcgtagaggatctggctagcgatgaccctgctgattggttcgctgaccatttccggggtgcggaacggcgttaccagaaactcagaag
gttcgtccaaccaaaccgactctgacggcagtttacgagagagatgatagggtctgcttcagtaagccagatgctacacaattaggcttgtac
atattgtcgttagaacgcggctacaattaatacataaccttatgtatcatacacatacgatttaggtgacactatagaatacacctgcaggacgt
cccaatgatcttaagttaa

### Example 2: Expression of a Bispecific Antibody Using the New Bi-Directional Dual-Expression 2-Vector Expression System Compared to the Conventional 4-Vector Expression System

Merck proprietary CHO host cell lines were used.

Plasmids were prepared by using QIAGEN kit (QIAGEN, Germany). Plasmids were transfected into CHO host cells through electroporation. After the recovery period, transfected cells were selected in appropriate selection media, CD-CHO (Thermo Fisher Scientific, Waltham, Mass.). The stable cells were then evaluated using a shake flask fed-batch process when viability reached ˜90%. Cells were seeded at 0.5×10⁶ viable cells/mL in chemically defined ActiPro™ medium (GE Healthcare Life Sciences, Logan, Utah). Chemically defined feeding media, CellBoost 7a and Cell Boost 7b (GE Healthcare Life Sciences, Logan, Utah) were supplemented daily into culture together with D-glucose (Sigma-Aldrich, St. Louis, Mo.). Glucose and lactate levels were measured everyday using the RANDOX RX imola chemistry analyzer (Crumlin, UK). Cell density and viability were measured using a Beckman Coulter ViCELL cell counter (Beckman Coulter, Indianapolis, Ind.). Antibody production levels and titers were determined by Protein-A HPLC (Waters, Mass.).

Determination of Gene Copy Number and mRNA Level of GOI

Genomic DNAs were extracted from CHO cultures using the DNeasy Blood and Tissue Kit (QIAGEN, Germany). Total RNAs from CHO cells were extracted using the RNeasy Plus Mini Kit from QIAGEN (QIAGEN, Germany). cDNAs were prepared from the RNA samples by reverse transcription using the SuperScript IV VILO Master Mix (Thermo Fisher Scientific, MA). Manufacturer recommended protocols were followed for the extraction of DNA/RNA, and for RNA reverse transcription. The QX200 Droplet Digital PCR (ddPCR) System (Bio-Rad, Hercules, Calif.) was used to determine the copy number of the heavy chain or light chain from genomic DNA, and to quantify the RNA transcript levels of the heavy chain or light chain from cDNA samples. Fluorescently-labeled oligo nucleotide probes for the ddPCR reactions were designed using the Primer Express Software (Applied Biosystems, Thermo Fisher Scientific, MA), and synthesized by Invitrogen (Thermo Fisher Scientific, MA).

Antibody Purification

Cell-free supernatants were adjusted to 500 μl in PBS and purified using PhyTips (PhyNexus, San Jose, Calif.; Cat: PTM 95-40-07). Briefly, tips were equilibrated in PBS, and washed in 10 mM Sodium Phosphate, 500 mM NaCl, pH 6.5 for three cycles. The captured antibody was eluted in 20 mM Sodium Acetate, pH 3.5. Protein concentration was assessed using UV spectroscopy (Spectramax M5e, Molecular Devices, San Jose, Calif.) at 260/280 nm wavelength.

Intact and Reduced HPLC

For intact HPLC, five micrograms of sample were injected into an Agilent 1290 UPLC system (Santa Clara, Calif.) using a Zorbax column: 300SB-C8 (Agilent; cat: 865750-906). Samples were detected using a wavelength of 214/280 nm with a 0.5 mL/min flow rate for 10 minutes. Empower was utilized to perform integration analysis of the detected peaks. Reduced HPLC was performed identically except that samples were reduced in 8 M Guanidinium chloride, 100 mM Tris HCL, 3 mM DTT, pH 8 and incubated at 56° C. for 20 minutes.

Size Exclusion Chromatography

Bispecific antibody assembly were assessed by using SEC chromatography. Purified protein materials were injected to a SEC column (Sepax) on an Agilent 1200 HPLC (Santa Clara, Calif.) with PBS as buffer. Collected peaks were analyzed using intact and reduced RP-LC-MS.

Results

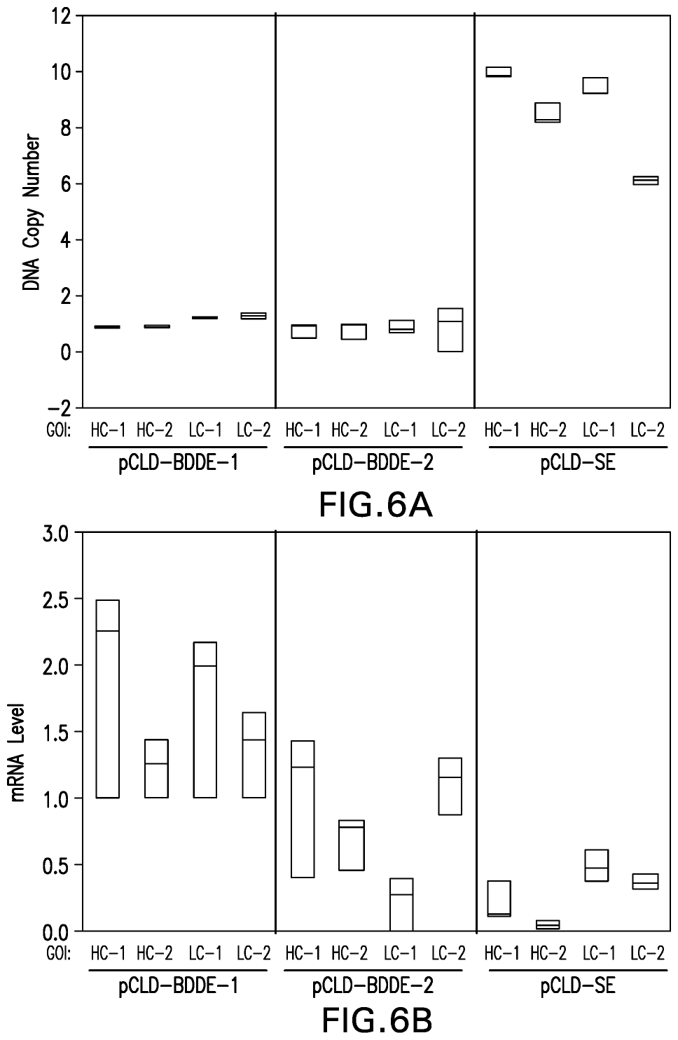

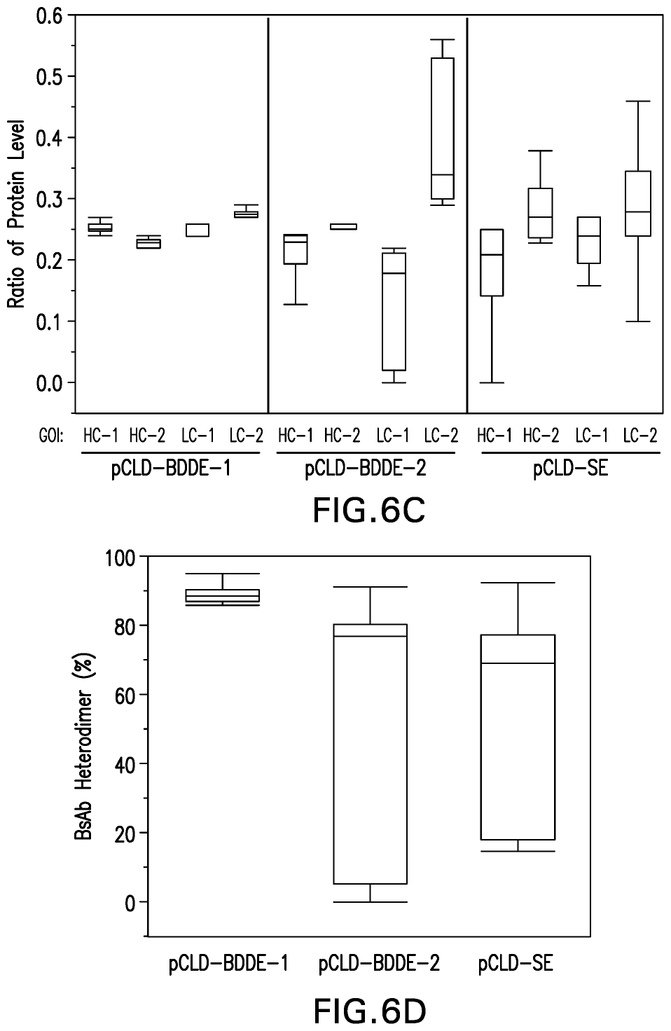

Compared to the conventional 4-vector system (i.e., each heavy chain or light chain of a bispecific antibody is encoded in an individual pCLD-SE vector), which lacks control of balanced expression of GOIs encoded in different vectors, the new bi-directional dual-expression 2-vector system using pCLD-BDDE-1 vectors (with insulators) was able to control four GOIs with similar DNA copy numbers after genomic integration (FIG. 6A) and similar mRNA levels (FIG. 6B). The new bi-directional dual-expression 2-vector system using pCLD-BDDE-2 vectors (without insulators) was not able to control four GOIs with similar mRNA levels (FIG. 6B). The 2-vector system using pCLD-BDDE-1 also achieved similar protein levels for four GOIs, compared to the 2-vector system using pCLD-BDDE-2 or the 4-vector system using pCLD-SE (FIG. 6C). Most surprisingly, the 2-vector system using pCLD-BDDE-1 was able to produce bispecific antibody heterodimers at a much higher percentage level with little variations, compared to the expression systems using pCLD-BDDE-2 or pCLD-SE (FIG. 6D).

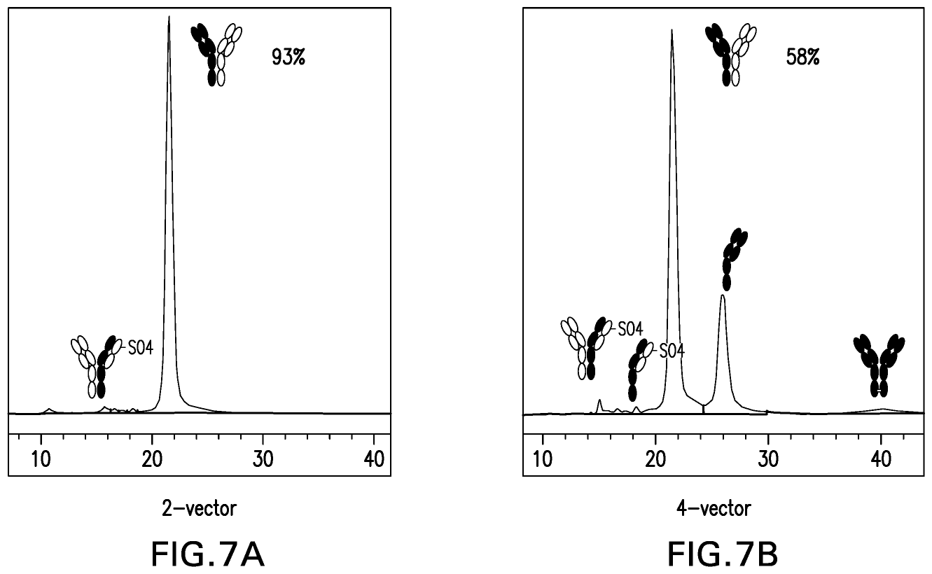

FIGS. 7A and 7B show the levels of the bispecific antibody heterodimer significantly increased and the impurity species decreased when using the new bi-directional 2-vector system, compared to the conventional 4-vector system. The stable pool material from the bi-directional 2-vector expression system improved and maintained the bispecific antibody assembly efficiency inside the cell and delivered high percentage of bispecific antibody heterodimer (>90%) at cell culture stage before purification (FIG. 7A). In contrast, the stable pool material from the conventional 4-vector expression system had less than 60% of bispecific antibody heterodimers and high percentage of impurity species, such as half molecules and homodimers (FIG. 7B).

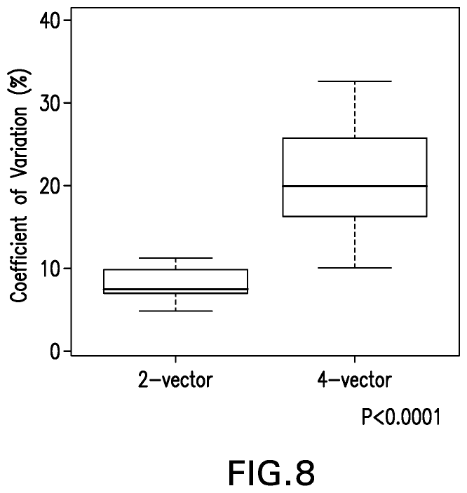

In addition, the bi-directional 2-vector expression system demonstrated high efficiency in bispecific heterodimer assembly for almost all clones from the stable pool with less clone-to-clone variation (FIG. 8). On the contrary, significant higher clone-to-clone variations have been found with the conventional 4-vector expression system (FIG. 8).

Evaluation of Production Scale and Process Conditions

To evaluate the impact of production scale and process conditions on product quality, 3 L bioreactor (Sartorius Stedim, Gottingen, Germany) and Ambr®250 bioreactor (Sartorius Stedim, Gottingen, Germany) have been used for fed-batch production. Cells were seeded at 0.5×10⁶ viable cells/mL in chemically defined ActiPro medium (GE Healthcare Life Sciences, Logan, Utah). CellBoost 7a and Cell Boost 7b (GE Healthcare Life Sciences, Logan, Utah) were supplemented daily into culture together with D-glucose (Sigma-Aldrich, St. Louis, Mo.). Glucose and lactate levels were measured daily using the RX imola (Imola, Crumlin, UK) for Ambr®250 and YSI Life sciences (Yellow Springs, Ohio) for the 3 L bioreactors. Cell density and viability were measured using a Cedex Hi Res Cell Counter (Roche Diagnostics, GmbH, Mannheim, Germany). Offline pH, pO₂ and pCO₂ was measured using ABL blood gas analyser (Radiometer, Denmark). Bioreactor pH was controlled at pre-defined set points using 1N NaOH or CO₂ sparging.

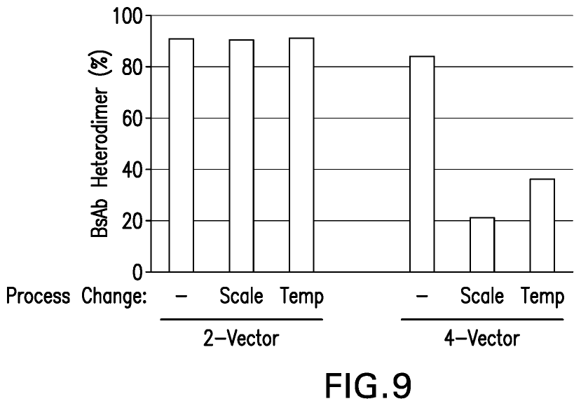

FIG. 9 demonstrates that the percentage of bispecific antibody heterodimer remained consistently high among different production batches with scale or temperature change for a stable clone comprising the bi-directional 2-vector system, whereas a top clone comprising the conventional 4-vector system is highly sensitive to the process condition change. The consistency of the bi-directional 2-vector system was observed with a scale range of 15 mL to 500 L and a temperature range of 32° C. to 37° C.

In summary, the above examples demonstrated that the bi-directional expression vector design can achieve balanced expression of the first GOI and the second GOI, and that the 2-vector expression system using two bi-directional expression vectors can significantly improve quantity and quality of bispecific antibody heterodimer production.

### SEQUENCE LISTING

The present application is being filed with a computer readable form (CRF) copy of the Sequence Listing. The CRF entitled 24689WOPCTSEQ.txt, which was created on Dec. 19, 2019 and is 90,619 bytes in size, is incorporated herein by reference in its entirety.

1-42. (canceled)

43. An expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) a DNA linker that connects the 5′ end of the first expression cassette and the 5′ end of the second expression cassette so that the first and the second expression cassettes are in the opposite directions;
(d) a third expression cassette comprising a second polynucleotide encoding a bacterial selectable marker; and
(e) a bacterial plasmid origin of replication;
wherein optionally the DNA linker is an insulator, a locus control region (LCR), a matrix attachment region (MAR), a scaffold attachment region (SAR), or an expression augmenting sequence element (EASE); and
wherein optionally the DNA linker is an EASE.

44. The expression vector of claim 43, further comprising a first insulator at the 3′ end of the first expression cassette and a second insulator at the 3′ end of the second expression cassette; and optionally further comprising two inverted terminal repeat (ITR) sequences flanking the portion of the expression vector comprising the first insulator, the first expression cassette, the DNA linker, the second expression cassette, and the second insulator.

45. The expression vector of claim 44, wherein
(a) the IRES comprises a polynucleotide sequence of SEQ ID NO:1, 2, 3, 25, 26, or 27;
(b) the eukaryotic selectable marker is a neomycin phosphotransferase, a histidinol dehydrogenase, a hygromycin B phosphotransferase, a xanthine-guanine phosphoribosyltransferase, a dihydrofolate reductase, a tryptophan synthetase, a puromycin N-acetyl-transferase, a thymidine kinase, an adenine phosphoribosyl transferase, a glutamine synthetase, an adenosine deaminase, or metallothionein-1;
(c) the first or the second promoter is a human cytomegalovirus (CMV) immediate-early promoter, a human elongation factor 1 alpha (EF1a) promoter, a SV40 promoter, a phosphoglycerate kinase 1 (PGK1) promoter, a human ubiquitin C (Ubc) promoter, a human β-actin promoter, a CAG promoter, a yeast transcription elongation factor 1 (TEF1) promoter, a yeast glyceraldehyde 3-phosphate dehydrogenase (GAPDH) promoter, or a yeast alcohol dehydrogenase 1 (ADH1) promoter;
(d) the first and the second insulators are HS4; or
(e) the ITR is piggyBac ITR;
wherein optionally the first and the second promoters are the same promoter; wherein optionally the first and the second promoters are a human CMV immediate-early promoter; wherein optionally the first and/or second expression cassette further comprises an enhancer; and wherein optionally the enhancer is a human CMV immediate-early enhancer.

46. The expression vector of claim 45, wherein the IRES comprises a polynucleotide sequence of SEQ ID NO:3.

47. The expression vector of claim 45, wherein the IRES comprises a polynucleotide sequence of SEQ ID NO:27.

48. An expression vector comprising:
(a) a first expression cassette comprising the following elements in the direction of 5′ to 3′: a first promoter operably linked to a first insertion site for a first gene of interest (GOI), an internal ribosome entry site (IRES), a first polynucleotide encoding a eukaryotic selectable marker, and a first polyadenylation (polyA) signal;
(b) a second expression cassette comprising the following elements in the direction of 5′ to 3′: a second promoter operably linked to a second insertion site for a second GOI, and a second polyA signal;
(c) an EASE that connects the 5′ end of the first expression cassette and the 5′ end of the second expression cassette so that the first and the second expression cassettes are in the opposite directions;
(d) a first insulator at the 3′ end of the first expression cassette and a second insulator at the 3′ end of the second expression cassette;
(e) a third expression cassette comprising a second polynucleotide encoding a bacterial selectable marker; and
(f) a bacterial plasmid origin of replication;
wherein optionally two inverted terminal repeat (ITR) sequences flanking the portion of the expression vector comprising (a)-(d).

49. An expression vector comprising a polynucleotide sequence of SEQ ID NO:4 or 5.

50. The expression vector of claim 49, wherein the expression vector comprises a polynucleotide sequence of SEQ ID NO:4.

51. The expression vector of claim 49, wherein the expression vector comprises a polynucleotide sequence of SEQ ID NO:5.

52. The expression vector of claim 48, wherein the first expression cassette further comprises the first GOI encoding a first polypeptide chain of a multi-chain recombinant protein.

53. The expression vector of claim 48, wherein the second expression cassette further comprises the second GOI encoding a second polypeptide chain of a multi-chain recombinant protein.

54. The expression vector of claim 48, wherein the first expression cassette further comprises the first GOI encoding a first polypeptide chain of a multi-chain recombinant protein, and wherein the second expression cassette further comprises the second GOI encoding a second polypeptide chain of the multi-chain recombinant protein.

55. The expression vector of claim 54, wherein the multi-chain recombinant protein is a bispecific antibody.

56. The expression vector of claim 55, wherein the first polypeptide chain of the multi-chain recombinant protein is a first heavy chain of the bispecific antibody, and wherein the second polypeptide chain of the multi-chain recombinant protein is a second heavy chain of the bispecific antibody.

57. The expression vector of claim 55, wherein the first polypeptide chain of the multi-chain recombinant protein is a first light chain of the bispecific antibody, and wherein the second polypeptide chain of the multi-chain recombinant protein is a second light chain of the bispecific antibody.

58. A host cell comprising the expression vector of claim 48, wherein the host cell is a mammalian host cell; and wherein optionally the mammalian host cell is a CHO cell.

59. A host cell comprising the expression vector of claim 48, wherein the host cell is a bacterial host cell.

60. A method of producing a multi-chain recombinant protein comprising a first polypeptide chain and a second polypeptide chain, comprising culturing the host cell of claim 58 under conditions in which the first polypeptide chain and the second polypeptide chain are expressed, and recovering the multi-chain recombinant protein comprising the first polypeptide chain and the second polypeptide chain from the culture, wherein the expression vector comprises the first GOI encoding the first polypeptide chain and the second GOI encoding the second polypeptide chain.

61. A method of propagating an expression vector, comprising culturing the host cell of claim 59 under conditions in which the expression vector is replicated, and recovering the expression vector from the culture.

62. A host cell comprising a first expression vector and a second expression vector that are the expression vector of claim 54, wherein the eukaryotic selectable marker of the first expression vector is different from the eukaryotic selectable marker of the second expression vector; wherein the first GOI and the second GOI of the first expression vector encode a first heavy chain and a second heavy chain of a bispecific antibody, respectively; and the first GOI and the second GOI of the second expression vector encode a first light chain and a second light chain of the bispecific antibody, respectively.

63. A method of producing a bispecific antibody, comprising culturing the host cell of claim 62 under conditions in which the first heavy chain, the second heavy chain, the first light chain, and the second light chain of the bispecific antibody are expressed, and recovering the bispecific antibody from the culture.
